(19) United States
(12) Patent Application Publication
(10) Pub. No.: US 2023/0013402 A1
(43) Pub. Date: Jan. 19, 2023

(54) REGENERATING FUNCTIONS AND PHENOTYPES OF CONNECTIVE TISSUE THROUGH NPAS2 SUPPRESSION

(71) Applicant: The Regents of the University of California, Oakland, CA (US)

(72) Inventors: Ichiro NISHIMURA, Venice, CA (US); Akishige HOKUGO, Los Angeles, CA (US); Hodaka SASAKI, Saitama (JP); Hiroko OKAWA, Los Angeles, CA (US); Lixin WANG, Los Angeles, CA (US); Robert D. DAMOISEAUX, Los Angeles, CA (US)

(73) Assignee: The Regents of the University of California, Oakland, CA (US)

(21) Appl. No.: 17/634,420

(22) PCT Filed: Sep. 4, 2020
(86) PCT No.: PCT/US2020/049529

§ 371 Date: Feb. 10, 2022

Related U.S. Application Data
(60) Provisional application No. 62/895,821, filed on Sep. 4, 2019.

(51) Int. Cl. A61K 31/4745; A61K 31/357; A61K 31/44; A61K 31/5377; A61K 31/165; A61K 31/365; A61K 31/36; A61K 31/428; A61K 31/33; A61K 31/519; C12N 15/113; A61K 31/4174; A61K 31/216; A61K 31/22; A61K 31/775; A61K 31/34; A61K 31/137; A61K 31/27; A61K 31/573; A61K 31/704; A61K 31/45; A61P 17/02

(52) U.S. Cl. CPC A61K 31/4745; A61K 31/357; A61K 31/44; A61K 31/5377; A61K 31/165; A61K 31/365; A61K 31/36; A61K 31/428; A61K 31/33; A61K 31/519; C12N 15/113; A61K 31/4174; A61K 31/216; A61K 31/22; A61K 31/775; A61K 31/34; A61K 31/137; A61K 31/27; A61K 31/573; A61K 31/704; A61K 31/45; A61P 17/02

(57) ABSTRACT

The present invention provides methods for improving or accelerating wound healing in a subject comprising administering to a wound of the subject in need thereof an agent that suppresses expression of a clock gene, wherein the clock gene is neuronal PAS domain protein 2 (Npas2). This invention also relates to methods for regenerating alveolar bone, regenerating connective tissue at a wound site, and for decreasing wound area size comprising administering to a bone loss site or a wound site, in particular, an open wound site, of a subject an agent that suppresses expression of Npas2.

### CROSS-REFERENCE TO RELATED APPLICATIONS

This application claims the benefit of and priority to U.S. Provisional Patent Application No. 62/895,821, filed Sep. 4, 2019, the contents of which are incorporated herein by reference in its entirety.

### FIELD OF THE INVENTION

This invention relates to methods for improving or accelerating wound healing in a subject comprising administering to a wound of the subject in need thereof an agent that suppresses expression of a clock gene, wherein the clock gene is neuronal PAS domain protein 2 (Npas2). This invention also relates to methods for regenerating alveolar bone comprising administering to a bone loss site of a subject in need thereof an agent that suppresses expression of Npas2. This invention further relates to methods for regenerating connective tissue at a wound site in a subject in need thereof comprising administering to the wound a therapeutically effective amount of a Npas2 expression suppressor. This invention also relates to methods for decreasing wound area size comprising topically administering to an open wound site of a subject an agent that suppresses expression of Npas2.

### BACKGROUND OF THE INVENTION

Unclosed open wound in skin and oral cavity presents a major threat for the current medical and dental treatment. The therapeutic goals of soft tissue wound management are infection control and wound closure. When Eliason and McLaughlin published a classic review in 1934 on postoperative wound complications, their focus was largely on surgical site infections. The effective use of antibiotics and aseptic procedures has significantly reduced the risk of surgical site infection today. However, the challenge to limit scarring remains problematic. Current approaches to achieve the wound closure employ sutures and adhesives (4) that have gone essentially unchanged for over a century.

The face and head are among the most frequent regions for wounding, which can occur due to accidents, assaults or battlefield injury. Facial wounds account for 4%-7% of all emergency department visits and the emergency department treats nearly 90% of facial soft tissue injuries, with a wide variety of wound closure methods available to clinicians. While major facial injuries, such as facial cancers, burns or fractures obviously lead to numerous social consequences for patients, even minor facial injuries can exhibit significant psychosocial impact, resulting in a decreased satisfaction with life, an altered perception of body image, and higher incidences of posttraumatic stress disorder, alcoholism, jail, unemployment or marital problems.

The primary legion of periodontitis in the oral cavity presents an open space between gingiva and the tooth surface, termed periodontal pocket, which provides an abnormal environment for oral microbiome, resulting in the growth of pathogenic bacteria. The closure of periodontal pocket is currently achieved only by pocket reduction surgery. The National Health and Nutrition Examination Survey of the U.S. civilian non-institutionalized population reported that 46% of dentate adults, representing 64.7 million people, suffered from periodontitis. The prevalence of periodontitis was positively associated with increasing age and with 8.9% of the people having developed severe or aggressive periodontitis. Similarly, periodontitis is the most widely experienced oral disease in companion dogs. According to the American Veterinary Dental College, periodontal disease is the most common clinical condition and the prevalence may reach over 90% in some dog breeds, presenting a large unmet veterinary patient population.

It is, therefore, of high importance to identify agents that can be administered to a wound of a subject, in particular an open dermal wound in the skin and oral cavity, to enhance wound healing, regenerate alveolar bone, and/or regenerate connective tissue at the wound site.

### SUMMARY OF THE INVENTION

In one aspect, this invention provides a method for improving or accelerating wound healing in a subject comprising administering to a wound of the subject in need thereof an agent that suppresses expression of a clock gene, wherein the clock gene is neuronal PAS domain protein 2 (Npas2).

In another aspect, this invention provides a method for regenerating alveolar bone comprising administering to a bone loss site of a subject in need thereof an agent that suppresses expression of Npas2.

In a further aspect, this invention provides a method for regenerating connective tissue at a wound site in a subject in need thereof comprising administering to the wound a therapeutically effective amount of a Npas2 expression suppressor.

In another aspect, this invention provides a method for decreasing wound area size comprising topically administering to an open wound site of a subject an agent that suppresses expression of Npas2.

Other features and advantages of the present invention will become apparent from the following detailed description, examples and figures. It should be understood, however, that the detailed description and the specific examples while indicating certain embodiments of the invention are given by way of illustration only, since various changes and modifications within the spirit and scope of the invention will become apparent to those skilled in the art from this detailed description. Whenever appropriate, any embodiment of the present invention can be combined with one or more other embodiments of the present invention, even though the embodiments are described under different aspects of the present invention.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1C show full-thickness skin punch wound healing is accelerated in Npas2−/− Mice. FIG. 1A is a standardized photograph of the skin wound that was obtained from 0 to 12 days after surgery, depicting the progressive wound closure and contraction. FIG. 1B shows the relative wound area that was calculated at 2, 4, 6, and 12 days. Npas2 KO mice showed a significantly smaller wound area than that of WT mice at day 12 (**P<0.01). FIG. 1C shows histological observation of wounds at Day 7 showed the formation of granulation tissue (GT) and the restoration of epithelial integrity (EP); however, the wound margin (dotted line) was clearly observed. At Day 14, the wound margin highlighted by hair follicles (HFs) was less clear and approached toward the granulation tissue (GT).

FIGS. 2A-2C show characterization of WT, Npas2+/−, and Npas2−/− skin fibroblasts. FIG. 2A shows the genotype of each fibroblast batch that was determined by genomic DNA PCR. The WT Npas2 allele generated a 250 bp PCR product, whereas the mutant allele generated a 350 bp PCR product. FIG. 2B shows the WST-1 assay demonstrated the increased cell proliferation rate in Npas2 KO fibroblasts (**P<0.01, significant difference compared with WT at the time points via the Tukey analysis). FIG. 2C shows the expression of core clock genes and the LacZ reporter gene was determined by RT-PCR every 6 for 48 hr (P value in the figure: two-way ANOVA for the interaction between the time and genotype factors. *P<0.05, **P<0.01, significant difference compared with WT at the time points via the Tukey analysis) (FIG. 2C).

FIGS. 3A-3H(c) show in vitro wound healing experiment using WT, Npas2+/− and Npas2−/− fibroblasts. FIG. 3A are images of time-lapse micrographs that captured the progressive scratch wound healing assay. FIG. 3B shows the number of migrated cells within the scratched area was significantly larger in the Npas2 KO groups at 12 hr and 24 hr (**P<0.01) FIG. 3C shows standardized images of floating collagen gel that depicted an increased collagen gel contraction in the Npas2 KO fibroblast groups. FIG. 3D shows that the area of collagen gels decreased over time. The gel contraction speed was faster in Npas2 KO fibroblasts (**P<0.01, significant difference shown only compared with WT). FIG. 3E is a schematic presentation of the FLECS-based single-cell contraction. FIG. 3F shows the ratio of contracted cells was increased in Npas2 KO fibroblasts. FIG. 3G shows that Npas2 KO mutation did not affect the gene expression of β-actin (Actb) and α-SMA (Acta2) in dermal fibroblasts. FIG. 3H(a)-3H(c) show the steady state gene expression level of integrin subunits αV (ItgaV), β3 (Itgb3), and β5 (Itgb5) in dermal fibroblasts was not affected by Npas2 KO mutation.

FIGS. 4A-4C show collagen synthesis by WT, Npas2+/− and Npas2−/− fibroblasts in vitro. FIG. 4A shows gene expression of collagen type I (Col1a1 and Col1a2), type III (Col3a1), type XII (Col12a1), and type XIV (Col14a1)(**P<0.01,*P<0.05, significant difference shown only compared with WT). FACIT collagen type XII and type XIV showed significantly increased steady-state mRNA levels in Npas2+/− and Npas2−/− fibroblasts. FIG. 4B shows images for cultured fibroblasts with picrosirius red staining highlighted the synthesis of collagen fibers. FIG. 4C shows the in vitro collagen fiber deposition was measured by picrosirius red staining (**P<0.01 by one-way ANOVA with post hoc Holm test).

FIGS. 5A-5C show the evaluation of collagen fiber structure in the wound healing area. FIG. 5A shows confocal laser scanning microscopy depicted the collagen fiber architecture stained with picrosirius red at 14 days after surgery. FIG. 5B shows measurement of the wound closure ratio using the wound closure area (WCA) calculated as the width of the ISA (a) between panniculus carnosus (PC) subtracted by the granulation tissue area (GT: b), which was normalized by ISA. FIG. 5C shows the wound closure ratio was greater in Npas2+/− and Npas2−/− mice at Day 14, albeit statistical significance was achieved only between the WT and Npas2−/− groups.

FIGS. 6A-6E show tooth extraction-induced alveolar bone regeneration in Npas2 KO mice. FIG. 6A shows C57B16J (B6) wild type (WT) mice were treated with maxillary left first molar extraction, which underwent wound healing of both oral mucosa and alveolar bone (arrow). Npas2 KO mice on B6 background demonstrated rapid wound closure and robust bone regeneration in the extraction socket. FIG. 6B shows MicroCT images of tooth-extraction wound healing at week 2. FIG. 6C shows MicroCT-based three-dimensional data analysis (BV/TV) for each of 3 root sockets that depicted the rapid bone filling in Npas2 KO mice in week 1 (W1) and week 2 (W2). Tukey's multiple comparison test, *: p<0.05; **: p<0.01 FIG. 6D shows in vitro mineralization of bone marrow MSC. After incubation in osteogenic medium for 28 days, MSC synthesized Alizarin Red-positive mineralization nodule area, which was significantly increased in Npas2 KO BMSC. FIG. 6E shows expression of BMP-2 by RT-PCR. Npas2 KO MSC (bone marrow derived mesenchymal stromal/stem cells) robustly increased BMP-2 expression after incubation in osteogenic medium. Statistical analyses: FIGS. 6C, 6D Tukey's multiple comparison test, *: p<0.05; **: p<0.01, FIG. 6E Student T test, **: p<0.01.

FIGS. 7A-7B show the effect of Reserpine on bone marrow stromal cells osteogenic differentiation. FIG. 7A shows wild type BMSC cultured in osteogenic medium supplemented with Reserpine (Dwn-C) demonstrated the increased alizarin red positive in vitro mineralization at culture day 21. Bars: Tukey analysis with p<0.05. FIG. 7B shows total RNA prepared from BMSC after 21 days of culture was subjected to real time RTPCR for osteopontin (Opn) and osteocalcin (Ocn) as well as house-keeping gene (Gapdh). The expression values were normalized with day 0 RNA.

FIG. 8 shows mouse dorsal skin punch was treated with Reserpine encapsulated DNV. FIGS. 9A-9C show periodontal tissue regeneration in the mouse periodontitis model. FIG. 9A shows periodontitis-induced alveolar bone resorption and MicroCT images of ligature induced mouse periodontitis. FIG. 9B is a flow diagram of a mouse model of ligature-induced periodontitis in which Reserpine+DNV was topically applied after ligature removal.

FIG. 9C shows the ligature placement induced severe inflammation, epithelial hyperplasia and connective tissue collagen disarrangement consistent with periodontitis. The ligature removal subsided the inflammatory reaction; however, epithelial and connective tissue abnormalities remained. The height of alveolar bone was unchanged (black arrow). In the Reserpine+DNV treatment group, epithelial and connective tissue were normalized There was a clear sign that alveolar bone was regenerated (between white and black arrows). The Reserpine+DNV treated group demonstrated the re-arrangement of gingival connective tissue collagen, similar to Control and the regeneration of alveolar bone also was observed.

FIG. 10 shows the titration assay for the top suppressor compound, DwnC. Dwn1 was serially diluted from 100 μM to 0.2 nM and applied to MSC Npas2-LacZ. Effective concentration (EC) was determined by LacZ expression and inhibitory concentration (IC) was determined by cell viability using Calcein AM/Hoechst 33342 staining DwnC and Dwn1 are both Reserpine.

FIGS. 11A-11D(b) show results of in vitro biological assays of Npas2 suppressing compound Dwn1 (Reserpine). FIG. 11A shows MSC in vitro mineralization was increased dose-dependently by Dwn1 supplementation. Dwn1 (1 μM) achieved an effect at a level similar to that of BMP-2 (100 ng/ml) supplementation FIG. 11B shows the expression of osteocalcin (OCN), whose expression level was increased as early as D21by Dwn1. FIG. 11C shows Dwn1 did not affect Bmal1 expression. FIGS. 11D(a)-11D(b) show Npas2+/− MSC responded to Dwn1 but Npas2−/− did not, suggesting the effect of Dwn1 was mediated by Npas2 suppression. **: p<0.01 to no treatment control by Tukey analysis

FIGS. 12A-12H show the effect of Dwn1 in the modified ligature-induced periodontitis in mice. FIG. 12A shows a 5.0 silk suture was placed at maxillary left second molar (M2) for 14 d and then removed. FIG. 12B shows gingival swelling indicated the ligature-induced periodontal inflammation. FIG. 12C shows RT-PCR of gingival tissue confirmed the inflammatory cytokine expression. FIG. 12D shows MicroCT demonstrating progressive alveolar bone loss. FIG. 12E shows deformable nano-scale vesicle (DNV) was applied to palatal gingiva using an oral appliance. Trans-oral mucosa drug administration was demonstrated by fluorescent-bisphosphonate on the alveolar bone. FIG. 12F shows Dwn1/DNV was applied to palatal gingiva after the suture was removed. Vehicle control showed abnormal epithelial thickening (white arrows). FIG. 12G shows MicroCT demonstrating the increased bone height in the Dwn1-treated palatal side but not in the untreated buccal side. FIG. 12H shows H&E (top row) and picrosirius red (bottom row) stained histological sections demonstrating the alveolar bone regeneration (top row) and gingival/PDL connective tissue reconstruction with Sharpey's fiber (SF) (bottom row). *: p<0.05; **: p<0.01

FIGS. 13A-13C show an unbiased chemical genetics analysis was used to determine the molecular mechanisms underlying implant osseointegration. FIG. 13A shows a flow diagram of chemical genetics analysis using BMSC carrying Npas2-LacZ reporter system. FIG. 13B shows high throughput screening of LOPAC1280 compounds for Npas2-LacZ expression of mouse BMSC. Hit compounds were identified as z-score >2.5 or <−2.5. FIG. 13C shows validation of Npas2-LacZ expression of hit compounds in triplicated experiments. The compounds (black bars) significantly modulated the Npas2-LacZ expression (p<0.05) compared to the untreated control (white bar) were identified.

FIGS. 14A-14D show Npas2 KO mice responded to bone wounding by bone regeneration. FIG. 14A shows a critical size calvarilal bone defect in C57B16J wild type (WT) and Npas2−/− mice on B6 background was treated with collagen sponge carrying 325 ng of BMP2 and was monitored by in vivo microCT for 4 weeks. FIG. 14 shows the regenerated bone volume in Npas2−/− mice was significantly larger than in WT mice. FIG. 14C shows tooth extraction-induced alveolar bone regeneration in Npas2 KO mice. WT mice were treated with maxillary left first molar extraction, which underwent wound healing of both oral mucosa and alveolar bone (arrow). Npas2 KO mice demonstrated rapid wound closure and robust bone regeneration in the extraction socket. FIG. 14D shows MicroCT data analysis (BV/TV) for each of 3 root sockets depicted the rapid bone filling in Npas2 KO mice in week 2. FIG. 14B Student T test, **: p<0.01 FIG. 14D Turkey's multiple comparison test, *: p<0.05; **: p<0.01

FIGS. 15A-15G show ligature-induced periodontitis in mice and alveolar bone regeneration in Npas2−/− mice after ligature removal. FIG. 15A shows ligature placement around maxillary 2^{nd} molar (M2) developed gingival inflammation (dotted line) over 14 days (D). FIG. 15B shows the expression of proinflammatory cytokines tissue such as IL-17a increased in the ligature placed side of palatal gingiva. FIG. 15C shows alveolar bone loss monitored by microCT progressively increased. FIG. 15D shows the gingival expression of Npas2 progressively increased. FIG. 15E shows at day 14, the ligature was removed, mimicking scaling and root plaining (SRP). At day 28, gingival inflammation was subsided. In WT mice, there was a gingival deficiency (white arrows) noted around M2, which was less visible in Npas2−/− mice. FIG. 15F shows before the suture removal at day 14, WT and Npas2−/− mice showed equivalent alveolar bone loss induced by periodontitis. FIG. 15G shows while alveolar bone height of WT mice remained low, Npas2−/− mice demonstrated increased bone height, suggesting bone regeneration. *: p<0.05; ***: p<0.001

FIGS. 16A-16D show a Npas2 suppressing compound (Dwn1) identified in HTS, regenerated alveolar bone. FIG. 16A shows the ligature was removed at D14 (FIG. 15E) and Dwn1 was topically applied to the palatal gingiva. At D28, the gingival defect seen in control mice (cont.) was less visible in Dwn1 treated mice. FIG. 16B shows the alveolar bone loss was attenuated at the palatal side where Dwn1 was applied. FIG. 16C. shows that the Dwn1 applied palatal side showed normalized gingiva at cement-enamel junction (white arrow) and new bone (red arrow) over the resorbed alveolar bone (black arrow). FIG. 16D shows that Dwn1 treatment showed normalized Sirius red stained gingival collagen arrangement with Sharpey's fiber (SF) under the epithelial (Ep) attachment on tooth to alveolar bone (B). *: p<0.05

FIG. 17 shows the HTS data were applied to Chemical Genomics analysis. Drug targets were largely overlapping within the chemical space of monoamine-related receptors, transporters and signal transduction pathways.

FIG. 18 shows MSC expressed neuronal monoamine transporters: vesicular monoamine transporter (VMAT); plasma membrane monoamine transporter (PMAT), extraneuronal monoamine transporter (EMT), dopamine transporter (DAT), serotonin transporter (SERT) and norepinephrine transporter (NET).

FIG. 19 shows Dwn1 (pan-monoamine transporter inhibitor) dose-dependently increased the in vitro mineralization at the similar level of BMP2 supplementation (100 ng/ml). **: p<0.01 against the control (white bar).

FIGS. 20A-20C show MSC behaviors of Npas2 KO mice. FIG. 20A shows MSC were exposed to osteogenic, chondrogenic and adipogenic differentiation media. Npas2−/− MSC exhibited increased multipotent differentiation capability than WT MSC. FIG. 20B shows the self-renewal activity was increased in Npas2−/− MSC. FIG. 20C shows the expression of stemness markers Nanog and KLF4 remained high in Npas2−/− MSC.

FIG. 21 shows hypertrophic scarring, which is characterized by deposits of excessive amounts of collagen (center) and a raised scar (left); the dense collagen fibers strongly stain blue with Masson Trichrome staining.

FIG. 22 shows a relationship between circadian rhythm and wound healing, adapted from Hoyle et al. Sci Transl Med., 2017, which is incorporated herein by reference in its entirety, who showed that human burn wounds that occurred during night time took much more time to heal than those that occurred during day time. Wound healing requires fibroblast (FBs) migration. To test fibroblast migration, Hyde et al. used an in vitro scratch model, that is a common method for in vitro wound healing. Skin FBs were cultured on a plate, and they scratched the plate at night time or day time. As shown, FBs scratched at day time migrated faster than FBs scratched at night time. This shows that faster migration means better healing.

FIG. 23 shows that Npas2, a clock molecule, has an important role in wound healing with implant Small titanium implants were surgically placed on rat femur. After 4 weeks, whole genome microarray of peri-implant tissue was performed. Npas2 was found to be is the most important clock molecule in the role of wound healing with implant. Npas2 knockout mutant mice were generated and implant surgery was performed in the same way as before (as described by Mengatto et al, PlosOne, 2011; Morinaga et al, Biomaterials, 2019, each of which is incorporated herein by reference in its entirety). In the wild type group, dense collagen tissue around the implant is beneficial for bone integration. Surprisingly, Npas2 knockout mice did not form dense collagen fibrous tissue. It was found that dense collagen fiber is the common structure of hypertrophic scarring. It was hypothesized that the suppression of Npas2 decreases “fibrosis” formation.

FIG. 24 shows that Npas knockout (KO) in mice improves wound healing and minimizes scarring in a mouse model of skin punch wound healing, adapted from Sasaki H, et al., Anat Rec (Hoboken). 2019, which is incorporated by reference herein in its entirety.

FIG. 25 shows the effect of Npas2 suppression on skin fibroblasts in vitro. Scratch wound healing and collagen gel contraction assay known as in vitro wound healing model were performed. Npas2 knockout fibroblasts show high cell migration and contraction ability. These results indicated Npas2 KO skin fibroblasts improve wound healing in vitro model.

FIG. 26 shows a platform to find Npas2 suppressive compounds. First, Mouse skin fibroblast with reporter gene were created and high throughput screening (HTS) was started with over one thousand FDA-approved compounds. After screening, 10 hit compounds that downregulate Npas2 were identified. One hit compounds downregulates Npas2 because of its toxicity. The cell viability assay was combined with HTS, and succeeded in eliminating False positives and obtained top 5 hit compounds.

FIG. 27 shows that Dwn1 accelerates fibroblast migration and gel contraction in vitro. The in vitro wound healing ability of Dwn1 was tested using a previously described method.

FIG. 28 shows that Dwn1 improved split wound healing with minimal scarring. To test in vivo wound healing using Dwn1, a 1.5 by 10 millimeter skin split model was created with a suture in the middle and three groups were designed. This model is similar to a clinical situation of a skin wound. When the skin was sutured only, visible wound healing was not effective. A suture and vehicle control with 10% DMSO, in the clinical observation, wound healing was visibly better than suture only. It is hypothesized that the moisture of vehicle may improve wound healing. In the third group, Dwn1 in 10% DMSO showed the best wound healing compared to the two other described groups.

FIG. 29 shows that Dwn1 improved split wound healing with minimal scarring. Masson trichrome staining was used to stain collagen deposition blue. Thick blue collagen deposition was found not only in granulation tissue but also in the peripheral wound. The histological staining for the vehicle control with 10% DMSO, was similar to control. Thick collagen deposition was observed. By contrast, Dwn1 in 10% DMSO showed a very small area of collagen deposition. These results indicated that Dwn1 improved split wound healing with minimal scarring.

FIG. 30 shows circadian rhythms inside cells are regulated by transcription-translation feedback loops of various clock genes including BMAL1, CLOCK and Npas2, which are basic helix-loop-helices, and Per or Cry genes, which are suppressor genes. (adapted from Sci Rep 8: 11996, 2018, and Morinaga et al, Biomaterials, 2019, each of which is incorporated herein by reference in its entirety). To date, these core circadian genes have been investigated as therapeutic target. However, BMAL1 or CLOCK deficient mice show abnormal phenotype or some critical phenomenon. On the other hand, Npas2 deficient mouse has not been reported any critical phenotype. Therefore, Npas2 is a safer molecular target.

FIG. 31 shows the mechanism of action for Reserpine (Res). Res blocks Vesicular Monoamine Transporters (VMAT) which are mostly expressed in neurons. (adapted from Endocrinology: Adult and Pediatric 2016, Science Direct, which is incorporated herein by reference in its entirety). Blockade of neuronal VMAT inhibits uptake of monoamine neurotransmitters such as norepinephrine, dopamine, serotonin and histamine in the synaptic vesicles. The relationship between monoamine neurotransmitter and circadian clock, transcription of the monoamine oxidase A (which maintains balance of monoamines) is regulated by the clock genes; BMAL1 and Npas2 and Per2. Per2 mutant mice showed reduced activity of monoamine oxidase A, as described by Hampp G. et al, Current Biology 2008, which is incorporated herein by reference in its entirety. This shows that the circadian clock regulates neurotransmitters.

FIGS. 32A-32B show a hypothesized mechanism for Reserpine (Res) and that serotonin can upregulate fibroblast function. (adapted from Wang C, et al. PloS ONE, 2013, which is incorporated herein by reference in its entirety). Although the mechanism of action for Reserpine in the skin has not been elucidated yet, it is hypothesized that Reserpine inhibits extra-neuronal monoamine transporter (EMT) which are similar to VMAT. When Res blocks EMT, monoamines, such as serotonin, will accumulate in the extracellular environment. It has been shown in previous study by Sadiq et al, Int J Mol Sci 2018, which is incorporated herein by reference in its entirety, that increased serotonin can upregulate fibroblast function. Thus, it is theorized that blockade of EMT by Reserpine accelerates Fibroblast (FB) function and wound healing.

FIGS. 33A-33F show linear wound/scar model of murine dorsal skin. FIG. 33A is a schematic of the animal model used in the study. Vertical wounds (10×1.5 mm) on both left and right side were made with a double-bladed scalpel. One ligation was performed at the center of the wound with 5-0 nylon suture. FIG. 33B shows Visual Analogue Scale (VAS) that was scored every day postoperatively until postoperative day 7 using gross images of the wounds/scars. FIG. 33C shows postoperative gross images of the wounds/scars with a ruler. Unit of the ruler is mm FIG. 33D shows histological images of center (left) and lateral (right) of wound/scar on postoperative day 7. Upper two were stained with Hematoxylin-eosin (HE). Lower two were stained with Masson's trichrome (MT). Yellow dotted lines indicate granulation tissue. Scale bar is 1000 μm. FIG. 33E shows a Scar Index that was evaluated using HE stained slices. FIG. 33F shows % area of fibrous tissue that was evaluated using MT stained slices. * shows p<0.05.

FIG. 34A-34C show selection and evaluation of candidate compound, Dwn1 for Npas2-suppression on dermal fibroblast in vitro. FIG. 34A shows a scatter plot of the high-through-put drug screening assay in vitro using FDA-approved compounds library in MSSR at UCLA. High absolute value of negative Npas2 Z score indicates that Npas2 expression was highly downregulated (X axis). High cell viability Z score indicates that fibroblast had high viability (Y axis). Candidate compound (Dwn1) were selected with the order from high absolute value of negative product of Npas2 Z score and highest viability. FIG. 34B shows an evaluation of circadian Npas2 expression in murine dermal fibroblasts treated with Dwn1 (1 μM or 10 μM) compared to control. FIG. 34C shows an evaluation of cell migration of murine dermal fibroblasts treated with Dwn1. * shows p<0.05.

FIGS. 35A-35B show effects of Dwn1 of collagen synthesis on murine dermal fibroblast in vitro. FIG. 35A shows Picrosirius red staining on murine dermal fibroblasts on day 7 after Dwn1 treatment. AA: 1-ascorbic acid. OD: optical density. CTRL: cell treated with control medium without AA. FIG. 35B shows gene expression of collagens (Col) type 1a1, 1a2, 3a1 and 14a1 on day 3 and 7 after Dwn1 treatment. * shows p<0.05.

FIGS. 36A-36E show effects of Dwn1 on the murine dorsal linear wound/scar model. FIG. 36A shows gross image on day 0 (D0), day 2 (D2), day 5 (D5), day 7 (D7) after surgery and starting topical application of Dwn1 to the wounds. Veh: vehicle. Vehicle or Dwn1+vehicle was applied every 24 hours postoperatively. FIG. 36B shows visual analogue score scale of wounds applied with vehicle or vehicle+Dwn1. FIG. 36C shows histological images of lateral wound/scar applied with vehicle or vehicle+Dwn1 on postoperative day 7. Yellow dotted lines indicate granulation tissue. Left two were stained with HE and right two were stained with MT. Scale bar is 1000 μm. FIG. 36D shows an evaluation of Scar Index using HE stained slices. FIG. 36E shows % Area of fibrous tissue was evaluated using MT stained slices. * shows p<0.05.

FIG. 37A-37C show molecular biological effects of Dwn1 on the murine dorsal linear wound/scar model. FIG. 37A shows a typical post-Laser capture microdissection (LCM) image. Slides were briefly stained with Hematoxylin and eosin before LCM. G: granulation tissue, W: wounded tissue. FIG. 37B shows gene expression of Col1a1, Col1a2, Col3a1), Col14a1, Tgfβ1 and Acta2 on granulation tissue (G) and wounded tissue (W). Gapdh was used as an internal control. * shows p<0.05. FIG. 37C shows immunohistochemical staining of αSMA in vivo at postoperative day 7 of wounds applied with vehicle or vehicle+Dwn1. Yellow dotted lines indicate granulation tissue. Scale bar is 100 μm.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Alveolar bone loss is a hallmark of periodontitis progression in humans and companion animals. The height of alveolar bone crest is located approximately 2 mm below the cementoenamel junction (CEJ) in healthy human subjects. The alveolar bone crest is subjected to bone resorption during the pathological development of periodontitis. Moderate and severe periodontitis conditions are defined as radiographic alveolar bone loss of 25%-50% and >50% of the root length, i.e., root tip to CEJ, respectively. Due to alveolar bone loss, which does not regenerate by conventional treatments, tooth extraction is often a likely clinical option.

The treatment options that can predictably regenerate the lost alveolar bone remain major clinical needs. The therapeutic stimulation of osteoblastic proliferation and differentiation has been investigated for bone regeneration and clinical applications of recombinant growth factors. The biological rationale of growth factor therapies lies in the embryonic and developmental processes. For example, mouse knockout mutations of the bone morphogenetic protein (BMP) signaling pathway molecules resulted in marked skeletal defects including spontaneous fractures and impaired fracture repair. Because the adult tissue regeneration undergoes, at least in part, reiterated embryonic and developmental processes, the application of growth factors is believed to induce the signaling pathway necessary for the bone regeneration in periodontal defects as well as for inducing the bone formation in extraction socket also known as socket preservation.

Current bone regenerative therapies utilize peptide biologics and growth factors, i.e., Emdogain® (porcine enamel matrix derivative product containing amelogenin); Infuse® (recombinant human BMP-2); GEM21S® (recombinant human platelet derived growth factor-bb); Fibroblast Growth Factor-basic 154 (recombinant human fibroblast growth factor-2); Forteo® (teriparatide, recombinant human N-terminal parathyroid hormone); rhGDF-5 (recombinant human growth differentiation factor-5, BMP-14, Phase 1/II completed).

In the past decades, recombinant peptide therapeutics played an important role in medical/dental practices and over 60 peptide drugs are approved in the US and other major markets. FDA has published a specific guideline for the safety monitoring of recombinant peptide products, which must include the stringent monitoring of adverse events related to anaphylactic reaction. If antibodies are generated against recombinant human peptides that share the sequence of endogenous proteins, they may potentially cause serious autoimmune reactions. The unexpected safety issues are the significant challenge to recombinant peptide therapeutics. In 2008, FDA has issued a black box warning for BMP-2 owing to the risk of overzealous inflammation. The additional safety monitoring may contribute to the time and cost of drug development. The current high cost as well as potential side effects of recombinant peptide products for dental regenerative therapy have become a significant hindering factor for the dental care delivery to our patients.

The circadian rhythm (also known as circadian clock), known as endogenous self-sustained and cell-autonomous oscillations of 24 hour rhythms in mammalian cells, is responsible for a wide range of physiological homeostasis functions, and the disruption of this rhythm is involved in chronic diseases, such as cardiovascular disease, diabetes, metabolic and sleep disorders, infertility, and impaired wound healing. A previous study reported that the database of human burn injuries showed that wounds injured during the night (the rest period) healed more slowly than wounds acquired during the day (the active period). Those results suggest a regulatory role of circadian rhythm in wound healing, albeit the mechanism of how the circadian rhythm contributes to skin wound healing is still unclear.

Circadian clock has been reported to regulate physiological tissue regeneration in adult animals Core circadian clock (rhythm) is rigidly maintained in the central brain by the suprachiasmatic nuclei (SCN) in the hypothalamus, which is the circadian pacemaker. Clock molecules: Clock, Npas2 and Bmal1 transcription factors induce the expression of Per and Cry genes, the protein products of which, in turn, inhibit Clock, Npas2 and Bmal1 transcriptional activity. In addition to core circadian clock (feed forward/back system in SCN), peripheral tissues such as bone, liver, skin and heart maintain their own circadian clock (e.g., clock molecule expression). Mouse calvarial bone organ culture demonstrated the bone mineral deposition in a circadian cycle. A microarray analysis of mouse calvaria revealed the presence of peripheral circadian rhythm in bone and that the daily expression of nearly 30% of all genes followed the 24-hour cycle, known as clock-controlled genes (CCG). Peripheral circadian clock is shown to play a regulatory role in cutaneous wound and bone fracture healing.

One of the circadian rhythm core regulators, neuronal PAS domain protein2 (NPAS2) is a member of the basic helix-loop-helix (bHLH)-PAS family of transcription factors and is a paralog of the circadian locomotor output cycles kaput (CLOCK). NPAS2 or CLOCK dimerizes with brain and muscle Arnt-like protein-1 (BMAL1) to regulate the gene transcription of two other circadian gene clusters; period (PER) and cryptochrome (CRY). PER and CRY then suppress the expression of NAPS2, CLOCK, and BMAL1 by a transcription/translation feedback loop system. Previous studies have revealed that Npas2 expression occurs in the mammalian forebrain and central brain but not in the SCN. However, the distinct expression of Npas2 was reported in peripheral tissue, including the heart, liver, vasculature, and skin.

Mouse skin fibroblasts have been reported to express Npas2, which might compensate for the lack of Clock expression. NPAS2 was identified among significantly upregulated genes in aging human skin by microarray analysis. Taken together, the inventors have hypothesized that Npas2 in skin fibroblasts plays a key role in homeostatic maintenance, and therefore Npas2 is a key factor during skin wound healing. The objective of the present study, as described in the Examples, was to address this hypothesis using Npas2 knockout mice.

Recently, ectopic upregulation of Npas2 in liver was linked to the fibrosis formation. Npas2 is an ortholog molecule of Clock and in the absence of Clock, Npas2 substitutes the peripheral clock function of fibroblasts. Therefore, fibrosis formation in peripheral tissues of Clock knockout mice may be contributed by pathological mechanism of substituting Npas2. It has been reported recently that Npas2 knockout (KO) mice exhibited much faster skin wound healing with minimal fibrosis.

Unless otherwise defined, all technical and scientific terms used herein have the same meaning as commonly understood by one of ordinary skill in the art to which this invention belongs. Further, unless otherwise required by context, singular terms shall include pluralities and plural terms shall include the singular.

The present invention is directed to the application of small molecule compounds targeting circadian clock molecule for regenerative therapy of alveolar bone. Circadian synchronization regulates numerous molecular, physiological and biological processes. Dysregulation of circadian rhythm was reported in neuropsychiatric diseases as well as in metabolic diseases and cancer. There are increasing reports suggesting that circadian clock molecules can be therapeutic targets; e.g., Bmal1 for malignant pleural mesothelioma and Alzheimer's disease. Therapeutic potential of small malecules modulating circadian systems has been proposed as a novel approach of “chronotherapy”. The present invention also is directed to small molecule-based chronotherapy for effective, safe and affordable dental tissue regeneration, including but not limited to alveolar bone regeneration, to patients in need thereof.

One of the major challenges in chronotherapy is selecting a target clock molecule. Because most, if not all, of cells possess circadian clock mechanisms, therapeutic modulation may result in a wide range of side effects. For example, KO mutations of Bmal1 or Clock generated various pathological phenotypes in peripheral bone tissues and premature aging symptoms (sarcopenia, cataracts, organ shrinkage). By contrast, Npas2 KO mutation did not result in embryonic and developmental pathology of jawbone, vertebral and appendicular bones. The level of Npas2 expression in SCN is low and has little contribution to the central circadian rhythm. Instead, increased Npas2 expression appears in peripheral tissues under disease states. The expression of Npas2 in bone tissue and MSC was significantly increased when exposed to titanium (Ti) biomaterial in vivo and in vitro, respectively, as described in Mengatto C M, et al., PLoS One. 2011; 6(1):e15848 and Hassan N, et al., PLoS One. 2017; 12(8):e0183359, respectively, each of which is incorporated by reference herein in its entirety. The Npas2 expression in peripheral tissues may be induced by “ad hoc” bases stimulated by environmental cues including wounding. The weighed gene co-expression analysis demonstrated that Npas2 was not co-regulated with other circadian clock genes, as described by Hassan N, et al., PLoS One. 2017; 12(8):e0183359, which is incorporated by reference herein in its entirety. Npas2 KO MSC maintained the normal expression of other core clock genes, as described by Morinaga K, et al., Biomaterials. 2018; 192:62-74, which is incorporated by reference herein in its entirety.

Provided herein are therapeutic methods of using agent(s) that suppresses expression of the clock gene neuronal PAS domain protein 2 (Npas2) (also called an Npas2 expression suppressor, Npas2 suppressor) for wound healing, in particular, for improving and/or accelerating repair and cure of the wound, for regenerating alveolar bone at a bone loss site, for regenerating connective tissue at a wound site and for decreasing wound area size at an open wound site, comprising administering to the open wound site and/or the bone loss site of a subject an Npas2 expression suppressor. The administered therapeutic agent(s) that suppress expression of Npas2 may be a chemical compound, a synthetic small interfering ribonucleic acid (siRNA) designed to target mRNA of a Npas2 gene, or a combination thereof.

The inventors of this application have found that Npas2 expression suppressor(s) regenerate connective tissue that has undergone a wound or chronic inflammation, regenerate dermal (skin) wounds and periodontal tissue wounds, and promote alveolar bone regeneration at a bone loss site.

In one aspect, this invention provides a method for improving or accelerating wound healing in a subject comprising administering to a wound of the subject in need thereof an agent that suppresses expression of a clock gene, wherein the clock gene is neuronal PAS domain protein 2 (Npas2).

In an embodiment, the administering is by a route selected from topical administration, transdermal administration and/or subcutaneous administration. In another embodiment, the wound is a dermal wound. In some embodiments, the dermal wound is a periodontal wound.

In certain embodiments, the periodontal wound comprises gingival connective tissue degeneration or alveolar bone resorption. In particular embodiments, the agent that suppresses expression of Npas2 accelerates human skin fibroblast migration in a cell migration assay.

In some embodiments, the agent that is a Npas2 expression suppressor is selected from norepinephrine, dopamine and serotonin uptake inhibitor, an oxidative phosphorylation inhibitor, a cyclooxygenase-2 inhibitor, a dopamine antagonist, or a central nervous system (CNS) stimulant.

In a particular embodiment, the agent that suppresses expression of Npas2 is an adrenergic uptake inhibitor that inhibits uptake of monoamine neurotransmitters norepinephrine (noradrenalin), dopamine and serotonin into presynaptic storage vesicles. In an embodiment, the norepinephrine, dopamine and serotonin uptake inhibitor is Reserpine, a catecholamine-depleting sympatholytic drug, which has the following chemical structure:

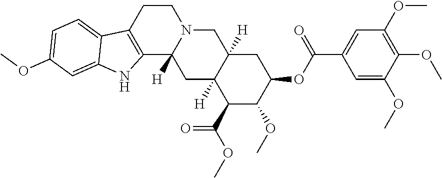

Reserpine is derived from Rauwolfia serpentine and other Rauwolfia species, and may be synthetically synthesized, as first described by Woodward R. B. et al., J. Am. Chem. Soc. 1956 78, 2023; and Tetrahedron 1958, 2, 1, or by alternate synthesis, e.g., as described more recently by Storck, G. et al, J. Am. Chem. Soc. 2005, 127, 16255-16262, which are incorporated by reference in their entirety. Reserpine irreversibly blocks the H+-coupled vesicular monoamine transporters, VMAT1 and VMAT2. Reserpine's blockade of VMAT2, which is expressed in neurons, inhibits uptake and reduces stores of the monoamine neurotransmitters norepinephrine, dopamine, serotonin and histamine in the presynaptic vesicles of neurons. Reserpine has been used as an antihypertensive, an antipsychotic drug, and a tranquilizer.

In additional embodiments, the agent that suppresses expression of Npas2 is one of the following Reserpine derivatives and analogs: rescinnamine, benzoyl reserpine, 3-methoxybenzoyl reserpine, 4-methoxybenzoyl reserpine, 3,4-dimethoxybenzoyl reserpine, 3,5-dimethoxybenzozyl reserpine, methylenedioxy reserpine, cinnamoyl reserpine, deserpidine, methyl reserpate, syrosingopine and evodiamine.

Rescinnamine also is obtained from Rauwolfia serpentine and other Rauwolfia species, and is used as an antihypertensive drug. Rescinnamine's pharmacological properties are similar to those of Reserpine, including sedative and hypotensive effects. In an embodiment, the agent that suppresses expression of Npas2 is Rescinnamine, which has the following chemical structure:

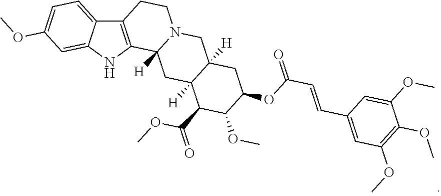

In another embodiment, the agent that suppresses expression of Npas2 is Benzoyl reserpine has the following chemical structure:

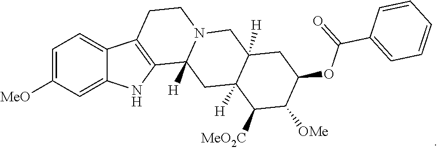 [SMILES: COC(=O)[C@H]1[C@H]2C[C@@H]3c4[nH]c5cc(OC)ccc5c4CCN3C[C@H]2C[C@@H](OC(=O)c2ccccc2)[C@@H]1OC]

In a further embodiment, the agent that suppresses expression of Npas2 is 3-methoxybenzoyl reserpine, which has the following chemical structure:

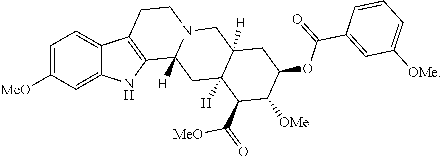 [SMILES: COC(=O)[C@H]1[C@H]2C[C@@H]3c4[nH]c5cc(OC)ccc5c4CCN3C[C@H]2C[C@@H](OC(=O)c2cccc(OC)c2)[C@@H]1OC]

In another embodiment, the agent that suppresses expression of Npas2 is 4-methoxybenzoyl reserpine, which has the following chemical structure:

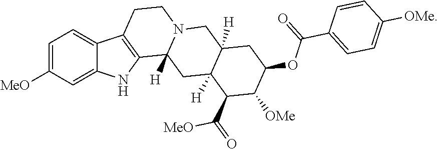 [SMILES: COC(=O)[C@H]1[C@H]2C[C@@H]3c4[nH]c5cc(OC)ccc5c4CCN3C[C@H]2C[C@@H](OC(=O)c2ccc(OC)cc2)[C@@H]1OC]

In an embodiment, the agent that suppresses expression of Npas2 is 3,4-dimethoxybenzoyl reserpine, which has the following chemical structure:

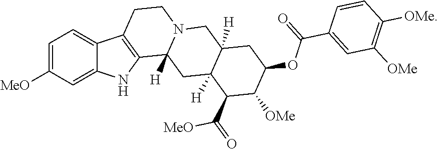 [SMILES: COC(=O)[C@H]1[C@H]2C[C@@H]3c4[nH]c5cc(OC)ccc5c4CCN3C[C@H]2C[C@@H](OC(=O)c2ccc(OC)c(OC)c2)[C@@H]1OC]

In another embodiment, the agent that suppresses expression of Npas2 is 3,5-dimethoxybenzozyl reserpine, which has the following chemical structure:

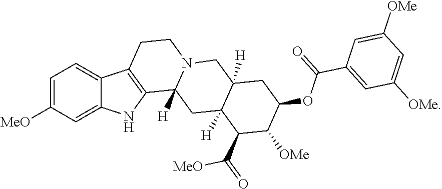 [SMILES: COC(=O)[C@H]1[C@H]2C[C@@H]3c4[nH]c5cc(OC)ccc5c4CCN3C[C@H]2C[C@@H](OC(=O)c2cc(OC)cc(OC)c2)[C@@H]1OC]

In still another embodiment, the agent that suppresses expression of Npas2 is methylenedioxy reserpine, which has the following chemical structure:

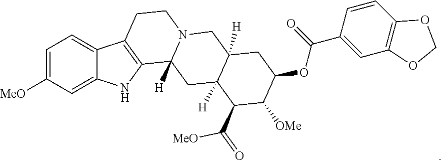 [SMILES: COC(=O)[C@H]1[C@H]2C[C@@H]3c4[nH]c5cc(OC)ccc5c4CCN3C[C@H]2C[C@@H](OC(=O)c2ccc3c(c2)OCO3)[C@@H]1OC]

In an embodiment, the agent that suppresses expression of Npas2 is Cinnamoyl reserpine, which has the following chemical structure:

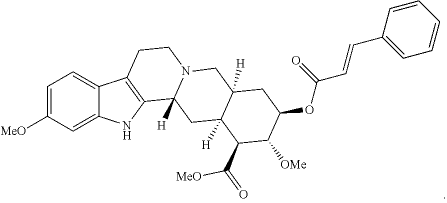 [SMILES: COC(=O)[C@H]1[C@H]2C[C@@H]3c4[nH]c5cc(OC)ccc5c4CCN3C[C@H]2C[C@@H](OC(=O)/C=C/c2ccccc2)[C@@H]1OC]

Deserpidine, also is a sympatholytic drug, i.e., it inhibits sympathetic nervous system, and has antihypertensive, sedative and antipsychotic properties; deserpidine, which is derived from Rauwolfia canescens L., and Apocyanaceae also may be synthesized from Reserpine. In certain embodiments, the agent that suppresses expression of Npas2 is deserpidine, which has the following chemical structure:

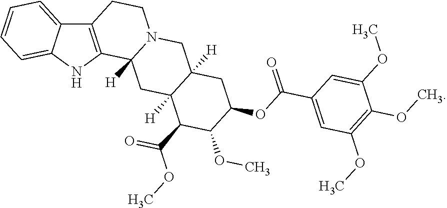 [SMILES: COC(=O)[C@H]1[C@H]2C[C@@H]3c4[nH]c5ccccc5c4CCN3C[C@H]2C[C@@H](OC(=O)c2cc(OC)c(OC)c(OC)c2)[C@@H]1OC]

In some embodiments, the agent that suppresses expression of Npas2 is methyl reserpate, which has the following chemical structure:

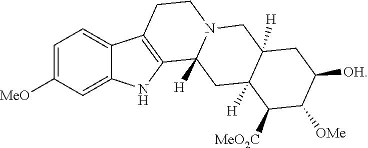 [SMILES: COC(=O)[C@H]1[C@H]2C[C@@H]3c4[nH]c5cc(OC)ccc5c4CCN3C[C@H]2C[C@@H](O)[C@@H]1OC]

In an embodiment, the agent that suppresses expression of Npas2 is syrosingopine, which has the following chemical structure:

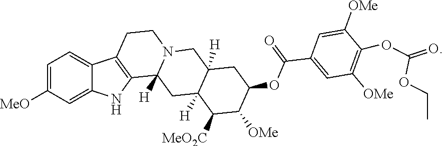 [SMILES: CCOC(=O)Oc1c(OC)cc(C(=O)O[C@@H]2C[C@@H]3CN4CCc5c([nH]c6cc(OC)ccc56)[C@H]4C[C@@H]3[C@H](C(=O)OC)[C@H]2OC)cc1OC]

In an embodiment, the agent that suppresses expression of Npas2 is evodiamine, which has the following chemical structure:

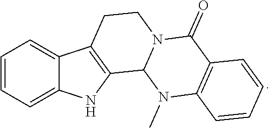 [SMILES: CN1c2ccccc2C(=O)N2CCc3c([nH]c4ccccc34)C21]

In another embodiment, the agent that suppresses expression of Npas2 is Tetrabenazine, which is an agent that is similar to Reserpine. Tetrabenazine reversibly inhibits VMAT2, which transports dopamine, serotonin, norepinephrine, and histamine into synaptic vesicles, causing decreased uptake of monoamines, as well as depletion of monoamine storage; Tetrabenazine reversibly depletes monoamines, particularly dopamine, by reversibly inhibiting monoamine uptake into vesicles of presynaptic neurons. Tetrabenazine has been used as an antipsychotic, and now is used the symptomatic treatment of various hyperkinetic disorders, chorea associated with Huntington's disease, and movement disorders, such as tardive dyskinesia, a side effect of antipsychotic medications. Tetrabenazine has the following chemical structure:

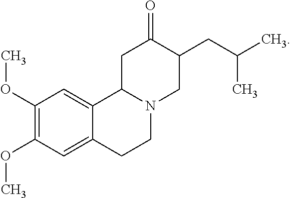 [SMILES: COc1cc2c(cc1OC)C1CC(=O)C(CC(C)C)CN1CC2]

In some embodiments, the agent that suppresses expression of Npas2 is a Tetrabenazine enantiomer or one of the eight stereoisomers of dihydrotetrabenazine, which also are VMAT2 inhibitors, the preparation of which is described in Yao, Z., et al., Eur J Med Chem. 2011 May; 46(5):1841-8, which is incorporated by reference herein in its entirety.

In an embodiment, the agent that suppresses expression of Npas2 is deutetrabenazine, which is an isotopic isomer of tetrabenazine in which six hydrogen atoms have been replaced by deuterium atoms. Deutetrabenazine has the following chemical structure:

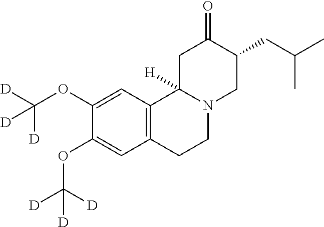 [SMILES: [2H]C([2H])([2H])Oc1cc2c(cc1OC([2H])([2H])[2H])[C@H]1CC(=O)[C@H](CC(C)C)CN1CC2]

Deutetrabenazine also inhibits vesicular monoamine transporter 2 (VMAT2) and is used for the treatment of chorea associated with Huntington's disease and tardive dyskinesia.

In additional embodiments, the oxidative phosphorylation inhibitor that is an agent that suppresses expression of Npas2 is Chlorpromazine. Chlorpromazine uncouples oxidative phosphorylation, but does not reduce norepinephrine and serotonin levels. Structurally unrelated to Reserpine, Chlorpromazine has the following chemical structure:

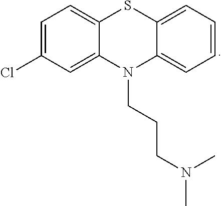 [SMILES: CN(C)CCCN1c2ccccc2Sc2ccc(Cl)cc21]

In another embodiment, the agent that suppresses expression of Npas2 is a Chlorpromazine analog, bromopromazine (Bromopromazine Hydrochloride), which has the following chemical structure:

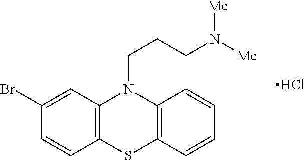 [SMILES: CN(C)CCCN1c2ccccc2Sc2ccc(Br)cc21 ; Cl]

1,4-thiazine-containing drugs similar to Chlorpromazine, include promethazine, trimeprazine, prochlorperazine, trifluoperazine, methotrimeprazine, and thioproperazine, having the following respective chemical structures (1)-(6):

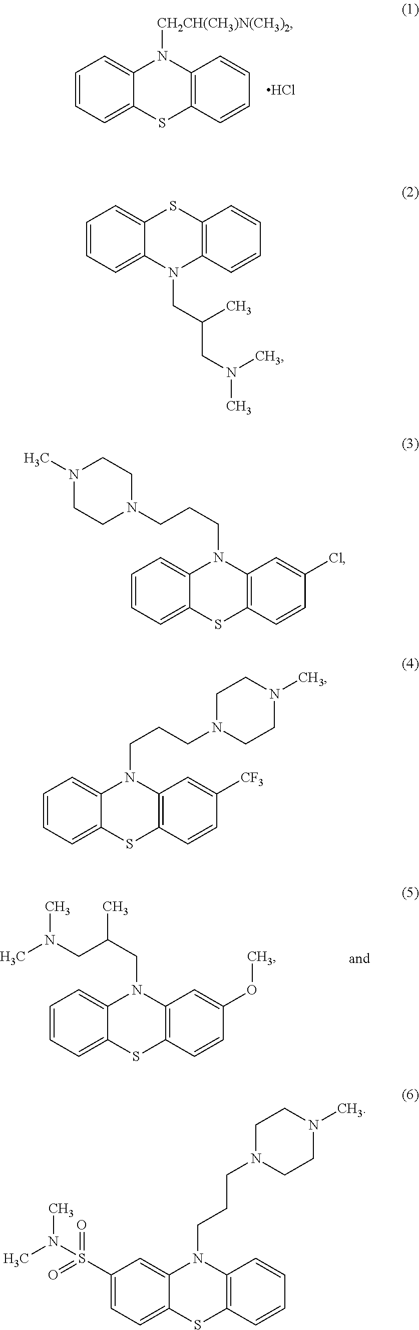

In some embodiments, the agent that suppresses expression of Npas2 is antimycin A, niflumic acid, molindone hydrochloride and mefexamide hydrochloride.

In certain embodiments, the Npas2 expression suppressing agent is Antimycin A, which has the following chemical structure:

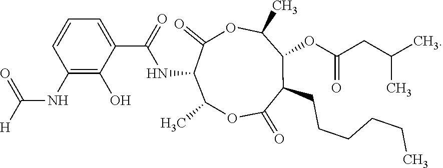 [SMILES: CCCCCC[C@H]1C(=O)O[C@H](C)[C@H](NC(=O)c2cccc(NC=O)c2O)C(=O)O[C@@H](C)[C@@H]1OC(=O)CC(C)C]

Antimycin A is produced by Streptomyces bacteria. Antimycin A is an inhibitor of oxidative phosphorylation and also disrupts the electron transport chain by inhibiting cytochrome c, thereby causing ATP production to stop. Antimycin A is used as a piscicide, a fish poison, in fisheries and in aquaculture to enhance catfish production by killing small and more sensitive fish species. Antimycin A, also known as Antimycin A1, is used as an antifungal agent, an insecticide and a miticide.

In an embodiment, the Npas2 expression suppressing agent is Antimycin A2, which has the following chemical structure:

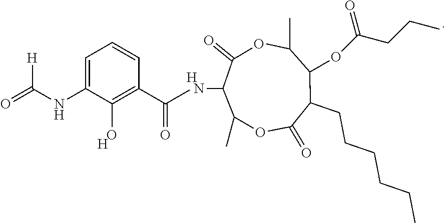

In some embodiments, the agent that suppresses expression of Npas2 is a derivative or analog of an Antimycin A, as described in US2005/0239873 (in particular, a 2-methoxy Antimycin A derivative); Batra, P. P., et al., J. Biological Chemistry, Vol. 246, No. 23, Issue of December 10, pp. 7125-7130, 1971 (in particular, Antimycin A di- and tri-acetates), Chevalier A., et al., Org. Lett. 2016, 18, 2395-2398 (in particular, an acylated Antimycin A derivative); Abidi, S. L., J. Chromatogr. 464 (1989) 453-458 (in particular, a homologue of Antimycin A or a methyl or dansyl derivative of Antimycin A) and Abidi, S L, J. Chromatogr. 447 (1988) 65-79 (in particular, subcomponents of Antimycin A, i.e., A1a, A1b, A2a, A2b, A3a, A3b, A4a, and A4b, and dansylated or methylated derivative of Antimycin A1a, A1b, A2a, A2b, A3a, A3b, A4a, and/or A4b), each of which is incorporated by reference herein in its entirety.

In an embodiment, the agent that suppresses expression of Npas2 is Antimycin A3 (also known as Blastmycin, and Blastomycin), which has the following chemical structure:

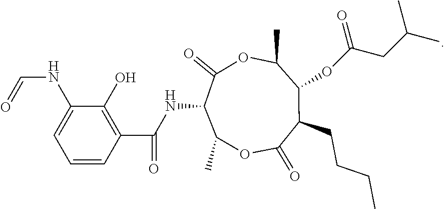 [SMILES: CCCC[C@H]1C(=O)O[C@H](C)[C@H](NC(=O)c2cccc(NC=O)c2O)C(=O)O[C@@H](C)[C@@H]1OC(=O)CC(C)C]

In another embodiment, the agent that suppresses expression of Npas2 is Antimycin A4, which has the following chemical structure:

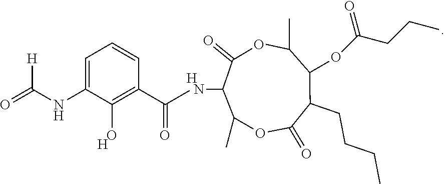

In an embodiment, Niflumic acid is the Npas2 expression suppressing agent; niflumic acid, which is a cyclooxygenase-2 inhibitor, has the following chemical structure:

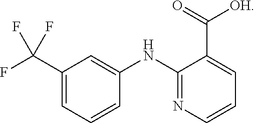 [SMILES: O=C(O)c1cccnc1Nc1cccc(C(F)(F)F)c1]

In another embodiment, the agent that suppresses expression of Npas2 is Talniflumate, a prodrug of Niflumic acid, which has the following chemical structure:

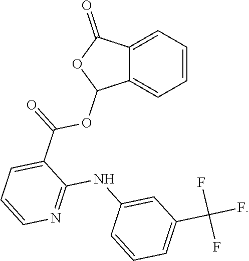 [SMILES: O=C1OC(OC(=O)c2cccnc2Nc2cccc(C(F)(F)F)c2)c2ccccc21]

In an embodiment, the agent that is a Npas2 expression suppressor is Molindone hydrochloride, an antipsychotic drug; Molindone hydrochloride is a dopamine D2/D5 receptor antagonist and has the following chemical structure:

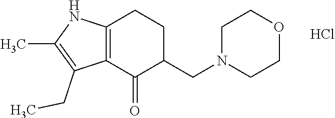 [SMILES: CCc1c(C)[nH]c2c1C(=O)C(CN1CCOCC1)CC2 ; Cl]

In another embodiment, the agent that is a Npas2 expression suppressor is Piquindone (Piquindone hydrochloride), a rigid analog of Molindone hydrochloride, which is an atypical antipsychotic drug that is a selective D2 receptor antagonist and has the following chemical structure:

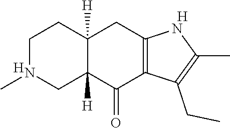

In an additional embodiment, the Npas2 expression suppressor is mefexamide hydrochloride (Mefexamide), a psychotherapeutic agent with central nervous system stimulatory action, which has the following chemical structure:

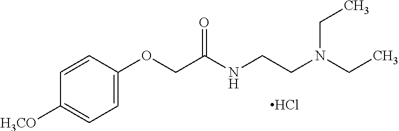 [SMILES: CCN(CC)CCNC(=O)COc1ccc(OC)cc1 ; Cl]

In various embodiments, the agent that suppresses expression of Npas2 is selected from econazole nitrate, Aceclofenac, Pravastatin, Tyloxapol, Isosorbide mononitrate, MS-1500387, (S)-(−)-Atenolol, Butenafine Hydrochloride, Aceclidine Hydrochloride, Atropine sulfate monohydrate, Trimethadione, Chlorphensin carbamate, Mafenide hydrochloride, Nifenazone, Articaine hydrochloride, Theobromine, Nifuroxazide, SAM001246626, Dropropizine (R,S), Diethylcarbamazine citrate, MS-1501214, Dolasetron mesilate, Estrone, Prednisolone, Daunorubicin hydrochloride, Cycloheximide, and Monensin sodium salt.

In an embodiment, the agent that suppresses expression of Npas2 is Aceclofenac which is a non-steroidal anti-inflammatory drug (NSAID) drug, that is an analog of Diclofenac. Aceclofenac has anti-inflammatory and analgesic properties and is used to treat rheumatoid arthritis, osteoarthritis, rheumatoid arthritis and ankylosing spondylitis. Aceclofenac inhibits the cyclo-oxygenase enzyme (COX). Aceclofenac has the following chemical structure:

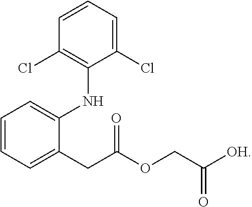 [SMILES: O=C(O)COC(=O)Cc1ccccc1Nc1c(Cl)cccc1Cl]

In another embodiment, the agent that suppresses expression of Npas2 is Pravastatin, which is a Hydroxymethylglutaryl-CoA (HMG-CoA) Reductase Inhibitor and is used as an anticholesteremic agent to lower plasma cholesterol and lipoprotein levels. Pravastatin has the following chemical structure:

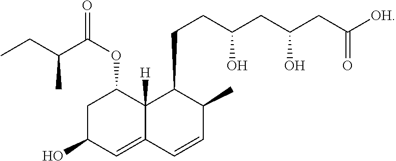 [SMILES: CC[C@H](C)C(=O)O[C@H]1C[C@H](O)C=C2C=C[C@H](C)[C@H](CC[C@@H](O)C[C@@H](O)CC(=O)O)[C@H]21]

In some embodiments, the agent that suppresses expression of Npas2 is Tyloxapol, which is a nonionic liquid polymer of the alkyl aryl polyether alcohol type. Tyloxapol is used as a nonionic surfactant used in bronchopulmonary studies of liquefaction and removal of mucupurulent secretions. Tyloxapol also has been shown to produce dose- and time-dependent cytotoxicity that induces apoptosis. Tyloxapol has the following chemical structure:

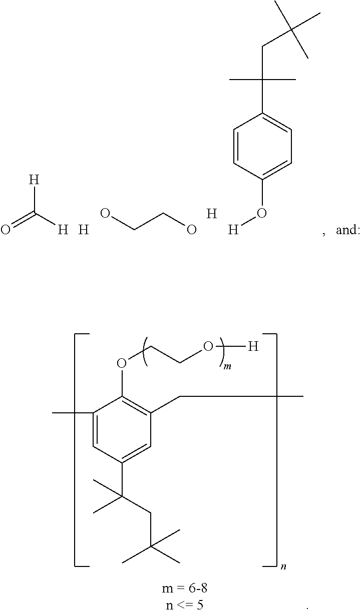

In an embodiment, the agent that suppresses expression of Npas2 is Isosorbide mononitrate, which is the mononitrate salt form of isosorbide, an organic nitrate with vasodilator activity; isosorbide mononitrate is used as a coronary artery vasodilator to treat angina and heart failure and also has been used to treat diffuse esophageal spasm. Isosorbide mononitrate has the following chemical structure:

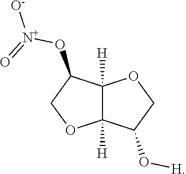

In another embodiment, the agent that suppresses expression of Npas2 is MS-1500387, also called Mercaptopurine, 6-Mercaptopurine, 6-MP and SPECTRUM1500387, which is an a purine antimetabolite, specifically, a thiopurine-derivative antimetabolite that is both an antineoplastic, i.e., an anticancer agent used to treat leukemia, such as acute lymphocytic leukemia and chronic lymphocytic leukemia, and is an immunosuppressive agent used to treat autoimmune diseases, such as ulcerative colitis. Mercaptopurine has the following chemical structure:

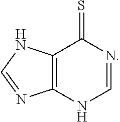

In certain embodiments, the agent that suppresses expression of Npas2 is(S)-(−)-Atenolol, the (S)-enantiomer of atenolol, also known as Esatenolol and (S)-Atenolol. (S)-(−)-Atenolol a beta-adrenergic antagonist, and is used as a beta blocker drug to treat high blood pressure, angina and to improve survival after a heart attack. (S)-(−)-Atenolol has the following chemical structure:

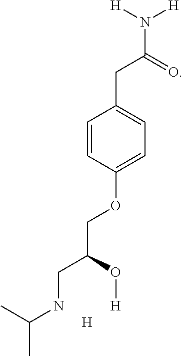

In another embodiment, the agent that suppresses expression of Npas2 is Butenafine Hydrochloride, which is the hydrochloride salt form of butenafine, a synthetic benzylamine; Butenafine Hydrochloride is an antifungal compound. Butenafine Hydrochloride interferes with the biosynthesis of ergosterol, an important component of fungal cell membranes, by inhibiting squalene epoxidase, an enzyme that is required for sterol formation needed for fungal cell membranes. Butenafine Hydrochloride has the following chemical structure:

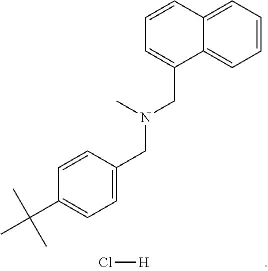 [SMILES: CN(Cc1ccc(C(C)(C)C)cc1)Cc1cccc2ccccc12 ; Cl]

In an additional embodiment, the agent that suppresses expression of Npas2 is Aceclidine Hydrochloride, also known as Glaucostat®, which is a non-selective muscarinic acetylcholine receptor partial agonist. Aceclidine Hydrochloride is used to treat narrow-angle glaucoma. Aceclidine Hydrochloride has the following chemical structure:

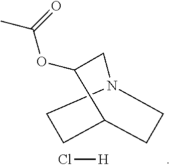

In an embodiment, the agent that suppresses expression of Npas2 is Atropine sulfate monohydrate, which is the sulfate salt of atropine, a naturally-occurring alkaloid isolated from the plant Atropa belladona L., Datura stramonium L., and other plants of Solanaceae family. Atropine functions as a sympathetic, competitive antagonist of muscarinic cholinergic receptors. Atropine Sulfate Monohydrate is a cholinergic receptor antagonist. Atropine sulfate monohydrate also acts as an antispasmodic agent, but does not exhibit any detectable effects on the central nervous system (CNS). Atropine sulfate monohydrate which has the following chemical structure:

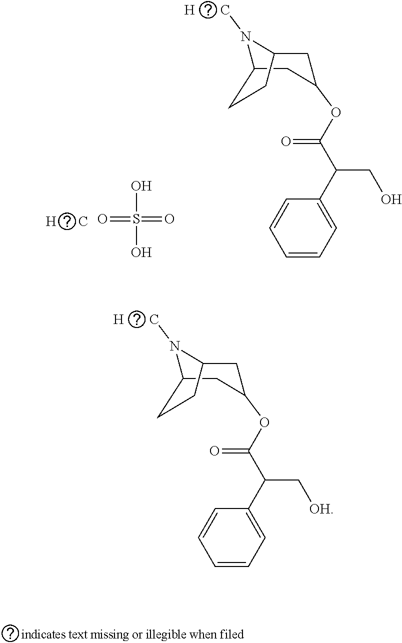

In some embodiments, the agent that suppresses expression of Npas2 is Trimethadione, which is an anticonvulsant compound that is used to treat epileptic conditions in patients who have used other medicines that did not work well; trimethadione has the following chemical structure:

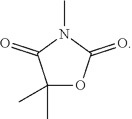

In various embodiments, the agent that suppresses expression of Npas2 is Chlorphensin carbamate, a centrally acting skeletal muscle relaxant that is used to treat muscle spasms; chlorphensin carbamate has the following chemical structure:

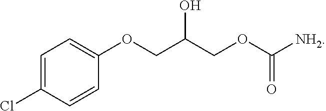 [SMILES: NC(=O)OCC(O)COc1ccc(Cl)cc1]

In an embodiment, the agent that suppresses expression of Npas2 is Mafenide hydrochloride, which has the following chemical structure:

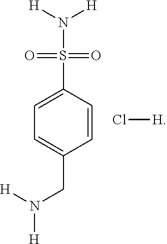

Mafenide hydrochloride is a sulfonamide drug that inhibits the enzyme, carbonic anhydrase; Mafenide hydrochloride is used as a topical antibiotic, particularly in burn therapy.

In an embodiment, the agent that suppresses expression of Npas2 is Articaine hydrochloride, which has the following chemical structure:

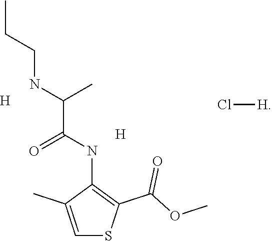

Articaine hydrochloride, the hydrochloride salt form of articaine is an amide-type local anesthetic that is used for pain relief in minor surgeries, typically in combination with epinephrine, a vasoconstrictor.

In an embodiment, the Npas2 expression suppressor is Nifenazone, which has the following chemical structure:

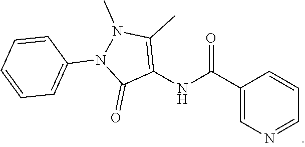 [SMILES: Cc1c(NC(=O)c2cccnc2)c(=O)n(-c2ccccc2)n1C]

Nifenazone is a non-steroidal anti-inflammatory drug, that also has analgesic, antipyretic and platelet-inhibitory therapeutic actions.

In another embodiment, the Npas2 expression suppressor is Theobromine, which has the following chemical structure:

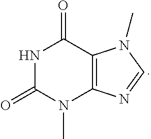

Theobromine (3,7-dimethylxanthine), is a purine alkaloid derived from the cacao plant; theobromine is an adenosine receptor antagonist and is used as a bronchodilator agent and as a vasodilator agent. Theobromine also has been used as a diuretic and as a heart stimulator.

In additional embodiments, the Npas2 expression suppressor is a compound that is structurally and pharmacologically similar to theobromine. In an embodiment, the theobromine related compound is theophylline, which has the following chemical structure:

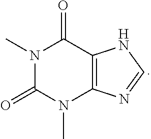

In another embodiment, the theobromine related compound is caffeine, which has the following chemical structure:

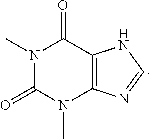

In an embodiment, the Npas2 expression suppressor is Nifuroxazide, an antibiotic that is used as an intestinal antibacterial agent to treat diarrhea and colitis in humans; nifuroxazide has the following chemical structure:

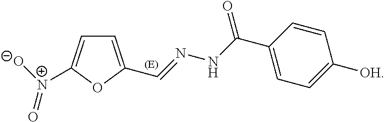 [SMILES: O=C(N/N=C/c1ccc([N+](=O)[O-])o1)c1ccc(O)cc1]

In some embodiments, the Npas2 expression suppressor is SAM001246626, also known as Atomoxetine hydrochloride, which has the following chemical structure:

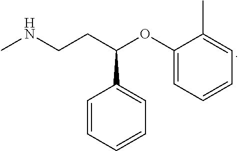 [SMILES: CNCC[C@@H](Oc1ccccc1C)c1ccccc1]

Atomoxetine hydrochloride is a norepinephrine reuptake inhibitor, which inhibits the pre-synaptic norepinephrine transporter, causing inhibition of the presynaptic reabsorption of norepinephrine and prolongation of norepinephrine activity in the synaptic cleft; atomoxetine hydrochloride is used to treat Attention Deficit Hyperactivity Disorder (ADHD).

In an embodiment, the Npas2 expression suppressor is Dropropizine (R,S), also known as dropropizine or dipropizine, which is a cough suppressant; dropropizine has the following chemical structure:

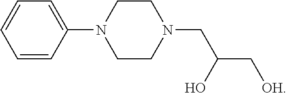 [SMILES: OCC(O)CN1CCN(c2ccccc2)CC1]

In another embodiment, the Npas2 expression suppressor is Diethylcarbamazine citrate, an anthelmintic drug used to treat filarial diseases; diethylcarbamazine citrate has the following chemical structure:

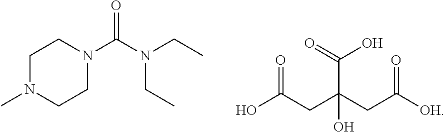

In a further embodiment, the Npas2 expression suppressor is MS-1501214, also known as enalapril maleate, which is the maleate salt form of enalapril. Enalapril maleate is an angiotensin-converting enzyme (ACE) inhibitor and is used to treat high blood pressure, congestive heart failure, kidney disease in diabetes, and has the following chemical structure:

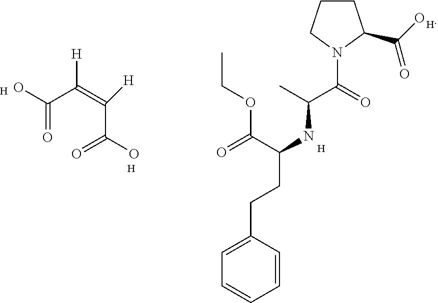

In an embodiment, the Npas2 expression suppressor is Dolasetron mesilate, also known as dolasetron mesilate, dolasetron (mesylate hydrate), and dolasetron. Dolasetron mesilate is a selective serotonin 5-HT₃ receptor antagonist with antiemetic activity and is used to treat nausea and vomiting after chemotherapy. Dolasetron mesylate hydrate has the following chemical structure:

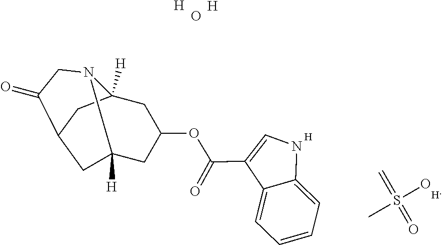

In another embodiment, the Npas2 expression suppressor is Estrone, also known as oestrone, which is a synthetically prepared or naturally occurring steroidal estrogen, specifically an agonist of the estrogen receptors ER-alpha and ER-beta. Estrone has the following chemical structure:

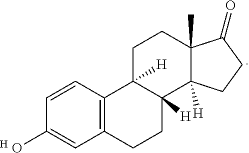

In an additional embodiment, the Npas2 expression suppressor is Prednisolone, which is a synthetic glucocorticoid with anti-inflammatory and immunomodulating properties; prednisolone acts as a corticosteroid hormone receptor agonist. Prednisolone has the following chemical structure:

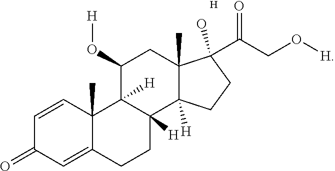

In an embodiment, the Npas2 expression suppressor is Daunorubicin hydrochloride, also known as daunorubicin and daunomycin, is the hydrochloride salt of an anthracycline antibiotic that has antineoplastic activity, which is used to treat leukemia, lymphoma and other cancers. Daunorubicin hydrochloride has the following chemical structure:

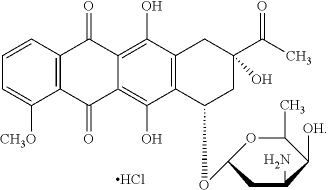

In some embodiments, the Npas2 expression suppressor is Cycloheximide, which is an antibiotic and an antibiotic fungicide produced by the bacterium Streptomyces griseus. Cycloheximide has the following chemical structure:

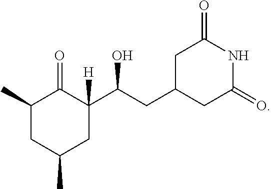 [SMILES: C[C@H]1C[C@@H](C)C(=O)[C@H]([C@@H](O)CC2CC(=O)NC(=O)C2)C1]

In another embodiment, the Npas2 expression suppressor is Monensin sodium salt, also known as Monensin sodium, is an antiprotozoal agent produced by Streptomyces cinnamonensis. Monensin sodium has the following chemical structure:

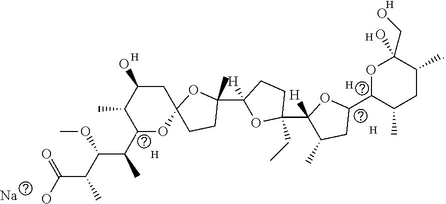

In an embodiment, the agent that suppresses expression of Npas2 is an oxidative phosphorylation inhibitor. In a particular embodiment, the oxidative phosphorylation inhibitor is Antimycin A, which has the following chemical structure:

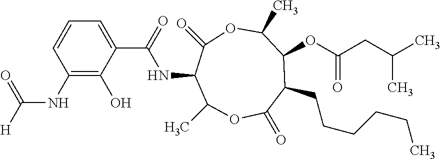 [SMILES: CCCCCC[C@H]1C(=O)OC(C)[C@@H](NC(=O)c2cccc(NC=O)c2O)C(=O)O[C@@H](C)[C@H]1OC(=O)CC(C)C]

In another embodiment, the agent is a Npas2 downregulating compound selected from the group consisting of a cytoskeleton/ECM inhibitor, a hormone agonist, a nitric oxide inhibitor, an intracellular Ca++ releasor, a kinase/phosphatase inhibitor, and a kinase inhibitor. In an embodiment, the cytoskeleton/ECM inhibitor is Brefeldin A, Colchicine, Podophyllotoxin or 5175348. In another embodiment, the hormone agonist is AC-93253 iodide, the nitric oxide inhibitor is Diphenyleneiodonium chloride, the intracellular Ca++ releasor is THAPSIGARGIN, the kinase/phosphatase inhibitor is PD-166285 hydrate, and the kinase inhibitor is PD-173952.

In a particular embodiment, the transdermal administration is an application to the wound of deformable nanoscale vesicles encapsulating the agent. In an embodiment, the transdermal administration is application to the wound of a transdermal delivery system selected from the group consisting of a microneedle coated with the agent, a solid polymer matrix having the agent incorporated therein, a transdermal patch comprising a reservoir storing the agent and a semi-permeable membrane, a transdermal gel comprising the agent dissolved therein, and a transdermal spray comprising the agent dissolved therein and a metered dose transdermal spray comprising the agent dissolved therein.

In certain embodiments, the agent is synthetic small interfering ribonucleic acid (siRNA) designed to target mRNA of a Npas2 gene. In some embodiments, the siRNA is administered by a route selected from the group consisting of microneedle array, electroporation, pressure, mechanical massage, cationic liposomes, cationic polymer-mediated delivery systems, ultrasound, conjugate delivery systems, microbubbles, liposomal bubbles, ultrasound sensitive nanobubbles, carbon nanotubes, lipid-based nanovectors, non-lipid organic-based nanovectors and inorganic nanovectors, gold nanoparticles, and gold nanorods. In various embodiments, the siRNA is chemically modified at a 2′ position of a ribose sugar ring, a phosphate backbone, a nucleobase and ribose sugar, 5′ termini modification or conjugation. In an embodiment, the ribose sugar ring is guanosine or uridine and the 2′ position modification is selected from the group consisting of 2′-OMe, 2′-F, 2′-O-methoxyethyl (2′-MOE). In another embodiment, the phosphate backbone is modified with phosphorodithioate, triazole dimers, amide or boranophosphate. In some embodiments, the nucleobase and ribose sugar modification is a 5-fluoro-2′-deoxyuridine (FdU), 2′-O-methyl phospshorodithioate (2′ O-MePS2), a lipophilic boron cluster, 3-N-[(1,12-dicarba-closo-dodecacarboran-1-yl)propan-3-yl]thymidine (C2B10H11, CB), thymidine and 5-bis(aminoethyl)-aminoethyl-2′-deoxyuridine. In a particular embodiments, the 5′ termini modification or conjugation is palmitic acid conjugation at the 5′ terminus of the siRNA, inverted thymidine (idT) coupling to the 3′ terminus of the siRNA and topalmitic acid conjugation at the 5′ terminus, conjugation of the siRNA with cell permeable peptide (CPPs), conjugation of the siRNA with aromatic compounds selected from the group consisting of phenyl, hydroxyphenyl, naphthyl, and pyrenyl derivatives; chemical modification at a 3′ overhang region with urea/thiourea bridged aromatic compounds; polyethylene glycol (PEG) conjugation at 3′ end of sense and anti-sense strands; and cholesterol conjugation of the siRNA.

In another aspect, this invention provides a method for regenerating alveolar bone comprising administering to a bone loss site of a subject in need thereof an agent that suppresses expression of Npas2. In some embodiments, the administering is by a route selected from topical administration, transdermal administration and/or subcutaneous administration. In an embodiment, the wound is a dermal wound. In another embodiment, the dermal wound is a periodontal wound. In a further embodiment, the periodontal wound comprises gingival connective tissue degeneration or alveolar bone resorption.

In an embodiment, the agent that suppresses expression of Npas2 accelerates human skin fibroblast migration in a cell migration assay. In some embodiments, the agent is selected from a norepinephrine and serotonin uptake inhibitor, an oxidative phosphorylation inhibitor, a cyclooxygenase-2 inhibitor, a dopamine antagonist, or a central nervous system (CNS) stimulant. In various embodiments, the agent is Reserpine. In some embodiments, the agent is antimycin A, niflumic acid, molindone hydrochloride and mefexamide hydrochloride. In certain embodiments, the agent is selected from econazole nitrate, Aceclofenac, Pravastatin, Tyloxapol, Isosorbide mononitrate, MS-1500387, (S)-(−)-Atenolo, Butenafine Hydrochloride, Aceclidine Hydrochloride, Atropine sulfate monohydrate, Trimethadione, Chlorphensin carbamate, Mafenide hydrochloride, Nifenazone, Articaine hydrochloride, Theobromine, Nifuroxazide, SAM001246626, Dropropizine (R,S), Diethylcarbamazine citrate, MS-1501214, Dolasetron mesilate, Estrone, Prednisolone, Daunorubicin hydrochloride, Cycloheximide, and Monensin sodium salt. In a particular embodiment, the agent is a Npas2 downregulating compound selected from the group consisting of a cytoskeleton/ECM inhibitor, a hormone agonist, a nitric oxide inhibitor, an intracellular Ca++ releasor, a kinase/phosphatase inhibitor, and a kinase inhibitor. In an embodiment, the cytoskeleton/ECM inhibitor is Brefeldin A, Colchicine, Podophyllotoxin or 5175348. In another embodiment, the hormone agonist is AC-93253 iodide, the nitric oxide inhibitor is Diphenyleneiodonium chloride, the intracellular Ca++ releasor is THAPSIGARGIN, the kinase/phosphatase inhibitor is PD-166285 hydrate, and the kinase inhibitor is PD-173952.

In particular embodiments, the transdermal administration is by deformable nanoscale vesicles encapsulating the agent. In certain embodiments, the transdermal administration is application to the wound of a transdermal delivery system selected from the group consisting of a microneedle coated with the agent, a solid polymer matrix having the agent incorporated therein, a transdermal patch comprising a reservoir storing the agent and a semi-permeable membrane, a transdermal gel comprising the agent dissolved therein, and a transdermal spray comprising the agent dissolved therein and a metered dose transdermal spray comprising the agent dissolved therein.

In various embodiments, the agent is synthetic small interfering ribonucleic acid (siRNA) designed to target mRNA of a Npas2 gene. In an embodiment, the siRNA is administered by a route selected from the group consisting of microneedle array, electroporation, pressure, mechanical massage, cationic liposomes, cationic polymer-mediated delivery systems, ultrasound, conjugate delivery systems, microbubbles, liposomal bubbles, ultrasound sensitive nanobubbles, carbon nanotubes, lipid-based nanovectors, non-lipid organic-based nanovectors and inorganic nanovectors, gold nanoparticles, and gold nanorods. In another embodiment, the siRNA is chemically modified at a 2′ position of a ribose sugar ring, a phosphate backbone, a nucleobase and ribose sugar, 5′ termini modification or conjugation. In some embodiments, the ribose sugar ring is guanosine or uridine and the 2′ position modification is selected from the group consisting of 2′-OMe, 2′-F, 2′-O-methoxyethyl (2′-MOE). In an embodiment, the phosphate backbone is modified with phosphorodithioate, triazole dimers, amide or boranophosphate. In another embodiment, the nucleobase and ribose sugar modification is a 5-fluoro-2′-deoxyuridine (FdU), 2′-O-methyl phospshorodithioate (2′ O-MePS2), a lipophilic boron cluster, 3-N-[(1,12-dicarba-closo-dodecacarboran-1-yl)propan-3-yl]thymidine (C2B10H11, CB), thymidine and 5-bis(aminoethyl)-aminoethyl-2′-deoxyuridine. In an embodiment, the 5′ termini modification or conjugation is palmitic acid conjugation at the 5′ terminus of the siRNA, inverted thymidine (idT) coupling to the 3′ terminus of the siRNA and topalmitic acid conjugation at the 5′ terminus, conjugation of the siRNA with cell permeable peptide (CPPs), conjugation of the siRNA with aromatic compounds selected from the group consisting of phenyl, hydroxyphenyl, naphthyl, and pyrenyl derivatives; chemical modification at a 3′ overhang region with urea/thiourea bridged aromatic compounds; polyethylene glycol (PEG) conjugation at 3′ end of sense and anti-sense strands; and cholesterol conjugation of the siRNA.

In certain embodiments, the transdermal administration is by deformable nanoscale vesicles encapsulating the agent. In some embodiments, the transdermal administration is application to the wound of a transdermal delivery system selected from the group consisting of a microneedle coated with the agent, a solid polymer matrix having the agent incorporated therein, a transdermal patch comprising a reservoir storing the agent and a semi-permeable membrane, a transdermal gel comprising the agent dissolved therein, and a transdermal spray comprising the agent dissolved therein and a metered dose transdermal spray comprising the agent dissolved therein.

In a further aspect, this invention provides a method for regenerating connective tissue at a wound site in a subject in need thereof comprising administering to the wound a therapeutically effective amount of a Npas2 expression suppressor. In particular embodiments, the administering is by a route selected from topical administration, transdermal administration and/or subcutaneous administration. In an embodiment, the wound is a dermal wound. In another embodiment, the dermal wound is a periodontal wound. In certain embodiments, the periodontal wound comprises gingival connective tissue degeneration or alveolar bone resorption. In various embodiments, the agent that suppresses expression of Npas2 accelerates human skin fibroblast migration in a cell migration assay. In certain embodiments, the agent is selected from a norepinephrine and serotonin uptake inhibitor, an oxidative phosphorylation inhibitor, a cyclooxygenase-2 inhibitor, a dopamine antagonist, or a central nervous system (CNS) stimulant. In particular embodiments, the agent is Reserpine. In some embodiments, the agent is antimycin A, niflumic acid, molindone hydrochloride and mefexamide hydrochloride. In an embodiment, the agent is selected from econazole nitrate, Aceclofenac, Pravastatin, Tyloxapol, Isosorbide mononitrate, MS-1500387, (S)-(−)-Atenolo, Butenafine Hydrochloride, Aceclidine Hydrochloride, Atropine sulfate monohydrate, Trimethadione, Chlorphensin carbamate, Mafenide hydrochloride, Nifenazone, Articaine hydrochloride, Theobromine, Nifuroxazide, SAM001246626, Dropropizine (R,S), Diethylcarbamazine citrate, MS-1501214, Dolasetron mesilate, Estrone, Prednisolone, Daunorubicin hydrochloride, Cycloheximide, and Monensin sodium salt. In another embodiment, the agent is a Npas2 downregulating compound selected from the group consisting of a cytoskeleton/ECM inhibitor, a hormone agonist, a nitric oxide inhibitor, an intracellular Ca++ releasor, a kinase/phosphatase inhibitor, and a kinase inhibitor. In an embodiment, the cytoskeleton/ECM inhibitor is Brefeldin A, Colchicine, Podophyllotoxin or 5175348. In another embodiment, the hormone agonist is AC-93253 iodide, the nitric oxide inhibitor is Diphenyleneiodonium chloride, the intracellular Ca++ releasor is THAPSIGARGIN, the kinase/phosphatase inhibitor is PD-166285 hydrate, and the kinase inhibitor is PD-173952. In particular embodiments, the transdermal administration is an application to the wound of deformable nanoscale vesicles encapsulating the agent. In an embodiment, the transdermal administration is application to the wound of a transdermal delivery system selected from the group consisting of a microneedle coated with the agent, a solid polymer matrix having the agent incorporated therein, a transdermal patch comprising a reservoir storing the agent and a semi-permeable membrane, a transdermal gel comprising the agent dissolved therein, and a transdermal spray comprising the agent dissolved therein and a metered dose transdermal spray comprising the agent dissolved therein.

In another embodiment, the agent is synthetic small interfering ribonucleic acid (siRNA) designed to target mRNA of a Npas2 gene. In a further embodiment, the siRNA is administered by a route selected from the group consisting of microneedle array, electroporation, pressure, mechanical massage, cationic liposomes, cationic polymer-mediated delivery systems, ultrasound, conjugate delivery systems, microbubbles, liposomal bubbles, ultrasound sensitive nanobubbles, carbon nanotubes, lipid-based nanovectors, non-lipid organic-based nanovectors and inorganic nanovectors, gold nanoparticles, and gold nanorods. In an embodiment, the siRNA is chemically modified at a 2′ position of a ribose sugar ring, a phosphate backbone, a nucleobase and ribose sugar, 5′ termini modification or conjugation. In another embodiment, the ribose sugar ring is guanosine or uridine and the 2′ position modification is selected from the group consisting of 2′-OMe, 2′-F, 2′-O-methoxyethyl (2′-MOE). In an embodiment, the phosphate backbone is modified with phosphorodithioate, triazole dimers, amide or boranophosphate. In another embodiment, the nucleobase and ribose sugar modification is a 5-fluoro-2′-deoxyuridine (FdU), 2′-O-methyl phospshorodithioate (2′ O-MePS2), a lipophilic boron cluster, 3-N-[(1,12-dicarba-closo-dodecacarboran-1-yl)propan-3-yl]thymidine (C2B10H11, CB), thymidine and 5-bis(aminoethyl)-aminoethyl-2′-deoxyuridine. In another embodiment, the 5′ termini modification or conjugation is palmitic acid conjugation at the 5′ terminus of the siRNA, inverted thymidine (idT) coupling to the 3′ terminus of the siRNA and topalmitic acid conjugation at the 5′ terminus, conjugation of the siRNA with cell permeable peptide (CPPs), conjugation of the siRNA with aromatic compounds selected from the group consisting of phenyl, hydroxyphenyl, naphthyl, and pyrenyl derivatives; chemical modification at a 3′ overhang region with urea/thiourea bridged aromatic compounds; polyethylene glycol (PEG) conjugation at 3′ end of sense and anti-sense strands; and cholesterol conjugation of the siRNA.

In particular embodiments, the connective tissue is one or more of collagen, dermis-like collagen fibers, or bone. In an embodiment, the wound site is a site of bone loss. In another embodiment, the bone loss is a site of periodontitis-induced alveolar bone resorption. In a further embodiment, the wound site is a site of gingival connective tissue degeneration.

In another aspect, this invention provides a method for decreasing wound area size comprising topically administering to an open wound site of a subject an agent that suppresses expression of Npas2. In certain embodiments, the administering is by a route selected from topical administration, transdermal administration and/or subcutaneous administration. In an embodiment, the wound is a dermal wound. In another embodiment, the dermal wound is a periodontal wound. In still another embodiment, the periodontal wound comprises gingival connective tissue degeneration or alveolar bone resorption. In an embodiment, the agent that suppresses expression of Npas2 accelerates human skin fibroblast migration in a cell migration assay. In particular embodiments, the agent is selected from a norepinephrine and serotonin uptake inhibitor, an oxidative phosphorylation inhibitor, a cyclooxygenase-2 inhibitor, a dopamine antagonist, or a central nervous system (CNS) stimulant. In another particular embodiment, the agent is Reserpine. In an embodiment, the agent is antimycin A, niflumic acid, molindone hydrochloride and mefexamide hydrochloride. In another embodiment, the agent is selected from econazole nitrate, Aceclofenac, Pravastatin, Tyloxapol, Isosorbide mononitrate, MS-1500387, (S)-(−)-Atenolo, Butenafine Hydrochloride, Aceclidine Hydrochloride, Atropine sulfate monohydrate, Trimethadione, Chlorphensin carbamate, Mafenide hydrochloride, Nifenazone, Articaine hydrochloride, Theobromine, Nifuroxazide, SAM001246626, Dropropizine (R,S), Diethylcarbamazine citrate, MS-1501214, Dolasetron mesilate, Estrone, Prednisolone, Daunorubicin hydrochloride, Cycloheximide, and Monensin sodium salt. In still another embodiment, the agent is a Npas2 downregulating compound selected from the group consisting of a cytoskeleton/ECM inhibitor, a hormone agonist, a nitric oxide inhibitor, an intracellular Ca++ releasor, a kinase/phosphatase inhibitor, and a kinase inhibitor. In a further embodiment, the cytoskeleton/ECM inhibitor is Brefeldin A, Colchicine, Podophyllotoxin or 5175348. In certain embodiments, the hormone agonist is AC-93253 iodide, the nitric oxide inhibitor is Diphenyleneiodonium chloride, the intracellular Ca++ releasor is THAPSIGARGIN, the kinase/phosphatase inhibitor is PD-166285 hydrate, and the kinase inhibitor is PD-173952. In various embodiments, the transdermal administration is an application to the wound of deformable nanoscale vesicles encapsulating the agent. In particular embodiments, the transdermal administration is application to the wound of a transdermal delivery system selected from the group consisting of a microneedle coated with the agent, a solid polymer matrix having the agent incorporated therein, a transdermal patch comprising a reservoir storing the agent and a semi-permeable membrane, a transdermal gel comprising the agent dissolved therein, and a transdermal spray comprising the agent dissolved therein and a metered dose transdermal spray comprising the agent dissolved therein. In another embodiment, the agent is synthetic small interfering ribonucleic acid (siRNA) designed to target mRNA of a Npas2 gene. In particular embodiments, the siRNA is administered by a route selected from the group consisting of microneedle array, electroporation, pressure, mechanical massage, cationic liposomes, cationic polymer-mediated delivery systems, ultrasound, conjugate delivery systems, microbubbles, liposomal bubbles, ultrasound sensitive nanobubbles, carbon nanotubes, lipid-based nanovectors, non-lipid organic-based nanovectors and inorganic nanovectors, gold nanoparticles, and gold nanorods. In an embodiment, the siRNA is chemically modified at a 2′ position of a ribose sugar ring, a phosphate backbone, a nucleobase and ribose sugar, 5′ termini modification or conjugation. In another embodiment, the ribose sugar ring is guanosine or uridine and the 2′ position modification is selected from the group consisting of 2′-OMe, 2′-F, 2′-O-methoxyethyl (2′-MOE). In a further embodiment, the phosphate backbone is modified with phosphorodithioate, triazole dimers, amide or boranophosphate. In another embodiment, wherein the nucleobase and ribose sugar modification is a 5-fluoro-2′-deoxyuridine (FdU), 2′-O-methyl phospshorodithioate (2′ O-MePS2), a lipophilic boron cluster, 3-N-[(1,12-dicarba-closo-dodecacarboran-1-yl)propan-3-yl]thymidine (C2B10H11, CB), thymidine and 5-bis(aminoethyl)-aminoethyl-2′-deoxyuridine. In certain embodiments, the 5′ termini modification or conjugation is palmitic acid conjugation at the 5′ terminus of the siRNA, inverted thymidine (idT) coupling to the 3′ terminus of the siRNA and topalmitic acid conjugation at the 5′ terminus, conjugation of the siRNA with cell permeable peptide (CPPs), conjugation of the siRNA with aromatic compounds selected from the group consisting of phenyl, hydroxyphenyl, naphthyl, and pyrenyl derivatives; chemical modification at a 3′ overhang region with urea/thiourea bridged aromatic compounds; polyethylene glycol (PEG) conjugation at 3′ end of sense and anti-sense strands; and cholesterol conjugation of the siRNA.

In another embodiment, the open wound site comprises connective tissue selected from one or more of collagen, dermis-like collagen fibers, or bone. In a further embodiment, the open wound site is a site of bone loss. In an embodiment, the bone loss is a site of periodontitis-induced alveolar bone resorption. In some embodiments, the open wound site is a site of gingival connective tissue degeneration.

In an embodiment the agent that suppresses expression of Npas2 and/or the agent that is a Npas2 downregulating compound is formulated as a pharmaceutical composition for topical administration, transdermal administration and/or subcutaneous administration. In particular embodiments, the pharmaceutical composition comprises a therapeutically effective amount of the agent that suppresses expression of clock gene Npas2, as described herein. In certain embodiments, the pharmaceutical composition comprises a therapeutically effective amount of the agent that suppresses expression of clock gene Npas2 effective to regenerate alveolar bone at a bone loss site, to regenerate connective tissue at a wound site, and/or to decrease wound area size of a wound site, in particular, an open wound site, of a subject in need thereof. In an embodiment, the pharmaceutical composition comprises at least one agent that suppresses expression of clock gene Npas2. In another embodiment, pharmaceutical composition comprises a combination of agents that suppresses expression of clock gene Npas2.

### Peripheral Circadian Clock Genes and Wound Healing

The function and phenotype of connective tissues vary in skin and oral tissue. Dermal fibroblasts, oral fibroblasts and bone forming osteoblasts are among connective tissue cells maintaining the site-specific function and phenotype, contributing to the homeostasis of health. Wounding in a broad sense affects connective tissue cells by modifying their phenotypes resulting in scarring or loss of functions. The inventors describe herein that peripheral circadian clock plays a previously unrecognized role during wound healing.

Circadian clock genes have been reported to regulate physiological tissue regeneration in adult animals. The core circadian clock is rigidly maintained in the suprachiasmatic nuclei (SCN) in the hypothalamus, which is the circadian pacemaker. Clock molecules: circadian locomotor output cycles kaput (Clock), Neuronal PAS domain 2 (Npas2) and aryl hydrocarbon receptor nuclear translocator-like (Arnt1, Bmal1) transcription factors induce the expression of period (Per) and cryptochrome (Cry) genes, the protein products of which, in turn, inhibit Clock, Npas2 and Bmal1 transcriptional activity. The circadian rhythm is responsible for a wide range of physiological homeostasis functions, and the disruption of this rhythm is involved in chronic diseases and impaired tissue repair.

In addition to the core circadian clock in SCN, peripheral tissues such as fibroblasts and osteoblasts have peripheral clocks that can function autonomously, as described by Matsui M S, Biological Rhythms in the Skin. Int J Mol Sci. 2016; 17(6), which is incorporated by reference herein in its entirety. A previous study reported that the database of human burn injuries showed that wounds injured during the night (the rest period) healed more slowly than wounds acquired during the day (the active period), as described by Hoyle N P, et al., Circadian actin dynamics drive rhythmic fibroblast mobilization during wound healing. Sci Transl Med. 2017; 9(415), which is incorporated by reference herein in its entirety. These results suggest a regulatory role of circadian rhythm in wound healing, though the mechanism of how the circadian rhythm contributes to skin wound healing is still unclear.

Mouse skin fibroblasts have been reported to express Npas2, which might compensate for the lack of Clock expression, as described by Landgraf D, et al., NPAS2 Compensates for Loss of CLOCK in Peripheral Circadian Oscillators. PLoS Genet. 2016; 12(2): e1005882, which is incorporated by reference herein in its entirety. Npas2 was identified among significantly upregulated genes in aging human skin by microarray analysis, as described by Glass D, et al., Gene expression changes with age in skin, adipose tissue, blood and brain. Genome Biol. 2013; 14(7):R75, which is incorporated by reference herein in its entirety. Recently, it has been observed that Npas2 plays a role in facilitating enhanced skin wound healing, as described by Sasaki H, et al., Neuronal PAS Domain 2 (Npas2)-Deficient Fibroblasts Accelerate Skin Wound Healing and Dermal Collagen Reconstruction. Anat Rec (Hoboken). 2019, which is incorporated by reference herein in its entirety.

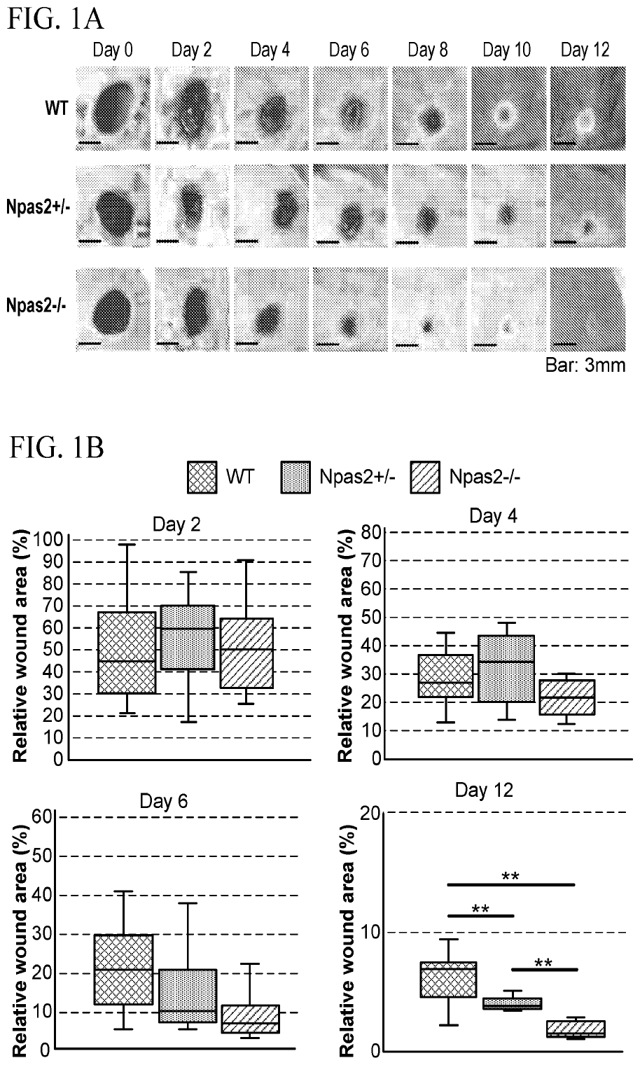

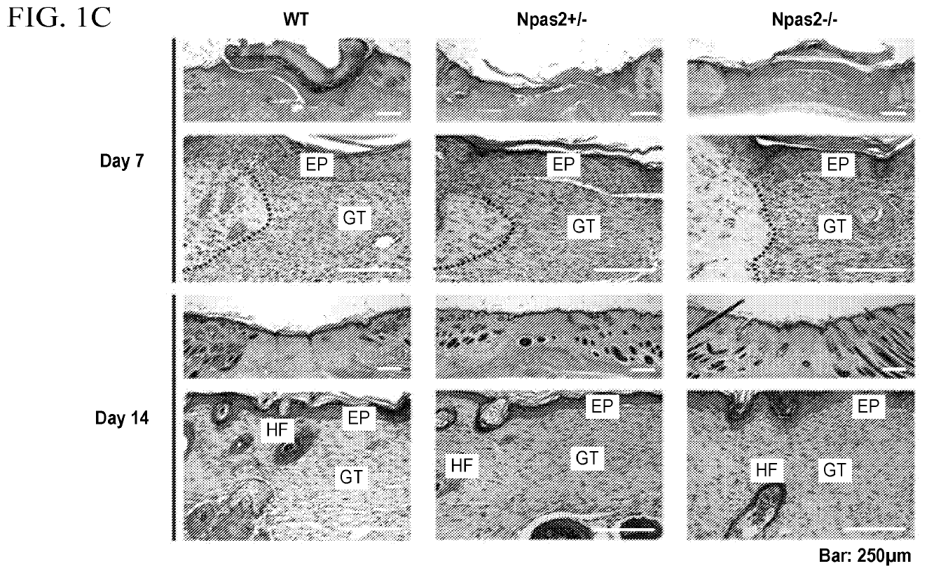

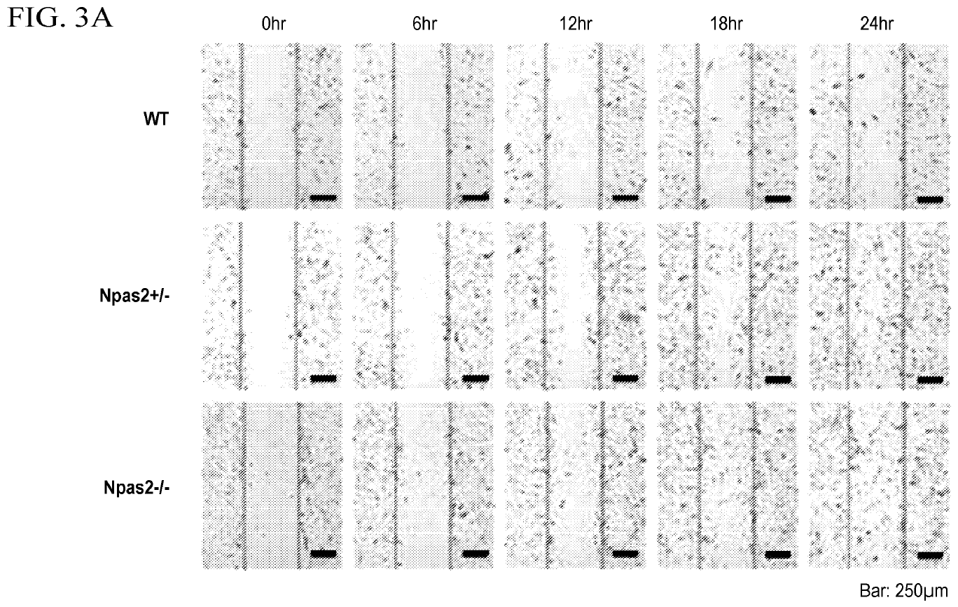

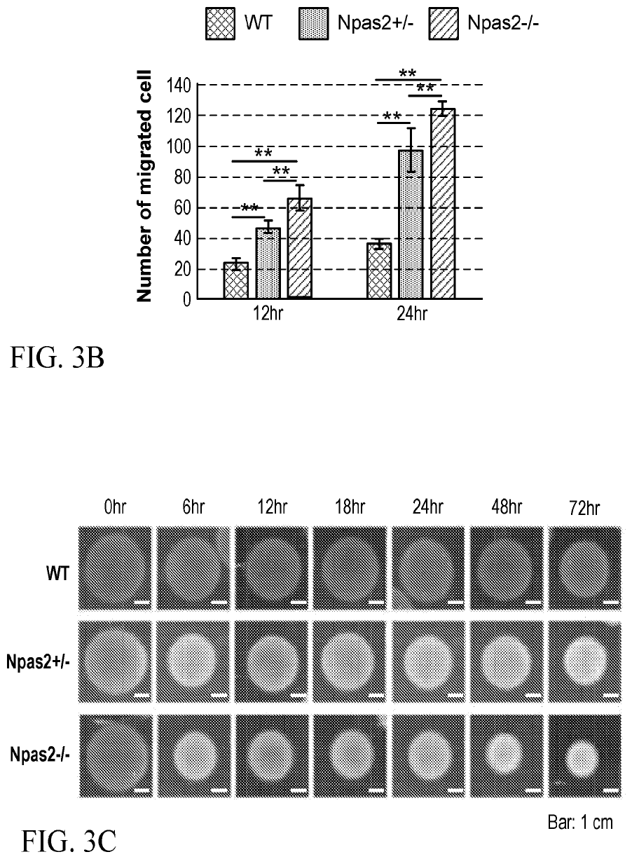

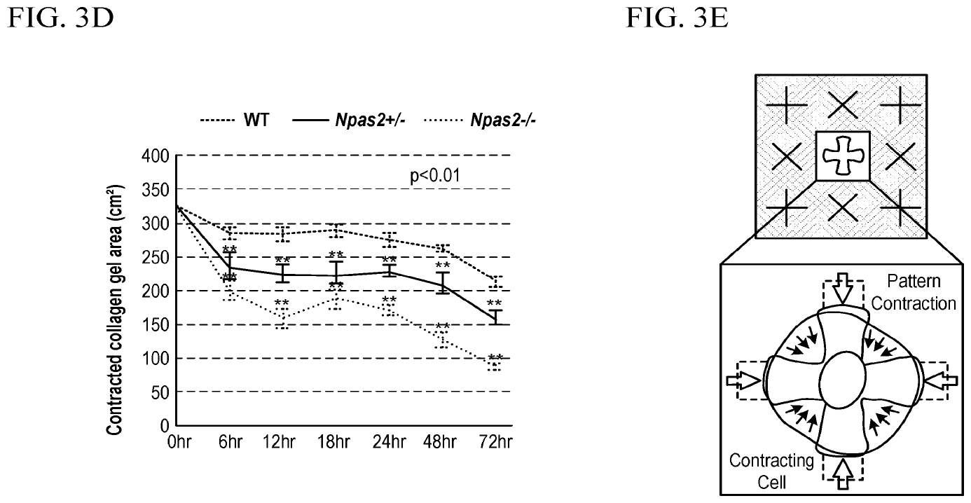

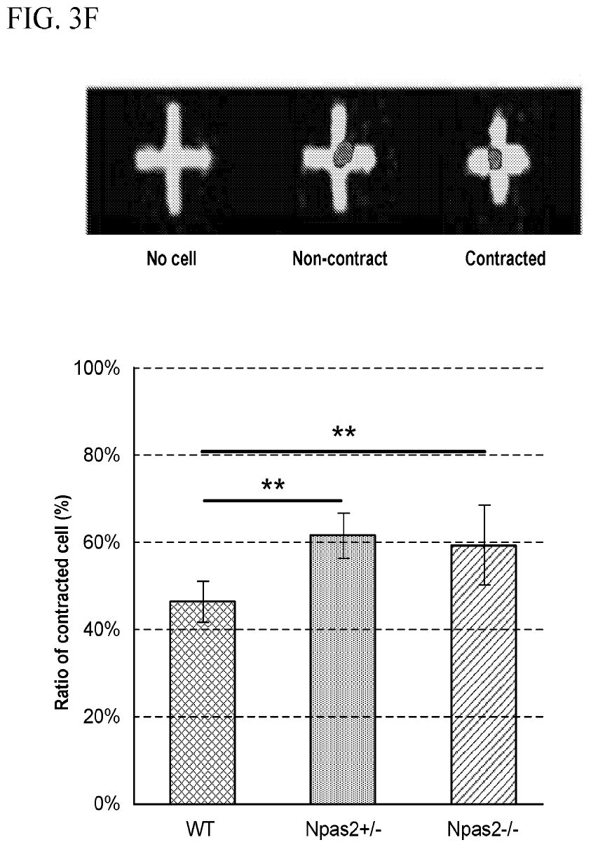

Npas2−/− mice demonstrated faster skin wound closure than the other groups (FIGS. 1A and 1B). Cell proliferation, cell migration and cell contraction of Npas2−/− fibroblasts were greater than in those for WT fibroblasts (p<0.01) (FIGS. 3A, 3B, 3C and 3D). An increased expression of type XII and XIV FAICT collagens and dermis-like collagen fiber formation was found in Npas2 KO fibroblasts in vitro. The collagen fiber structure in the granulation tissue area was better reconstructed in Npas2−/− mice. These data suggest that circadian rhythm, in particular Npas2, may regulate skin wound healing. These observations have provided the rationale to explore a novel opportunity for therapeutic development.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment”, “treating,” or “therapy” (as well as different forms thereof) of a disease-state in a mammal, particularly in a human, are used interchangeably herein and refer to (a) preventing the disease-state from occurring in a mammal, i.e., prophylaxis of the disease-state, in particular, when such mammal is predisposed to the disease-state but has not yet been diagnosed as having it; (b) inhibiting the disease-state, i.e., arresting its development, and/or curing the disease-state; and/or (c) relieving the disease-state, i.e., causing regression of the disease state. The term “treating” as used herein includes alleviating or reducing at least one adverse or negative effect or symptom of a condition, disease or disorder.

In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

As used herein, the terms “administering,” “administer,” or “administration” refer to delivering one or more compounds or compositions to a subject parenterally, enterally, or topically. In one embodiment, the compositions are applied locally. In another embodiment, the compositions are applied systemically. Administration can be accomplished to cells or tissue cultures, or to living organisms, for example humans Illustrative examples of parenteral administration include, but are not limited to, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. Illustrative examples of enteral administration include, but are not limited to oral, inhalation, intranasal, sublingual, and rectal administration. Illustrative examples of topical administration include, but are not limited to, transdermal and vaginal administration. In particular embodiments, an agent or composition is administered parenterally, optionally by intravenous administration or oral administration to a subject.

The terms “subject,” “individual,” and “patient” are used interchangeably herein, and refer to an animal, for example a human, to whom treatment, including prophylactic treatment and inhibition of the disease-state and secondary infection, with an agent that suppresses expression of Npas2, as described herein, and/or pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

According to any of the methods of the present invention and in one embodiment, a subject as described herein is human. In another embodiment, the subject is non-human. In one embodiment, the subject is a vertebrate. In another embodiment, the subject is a mammal. In another embodiment, the subject is a primate, which in one embodiment, is a non-human primate. In another embodiment, the subject is murine, which in one embodiment is a mouse, and, in another embodiment is a rat. In another embodiment, the subject is a canine, feline, bovine, equine, caprine, ovine, porcine, simian, ursine, vulpine, or lupine. In one embodiment, the subject is a chicken or fish.

In one embodiment, a composition of the present invention comprises a pharmaceutically acceptable composition. In an embodiment, the composition comprises an agent that suppresses expression of a clock gene, wherein the clock gene is neuronal PAS domain protein 2 (Npas2). In particular embodiments, the agent that suppresses expression of Npas2 is any one of the agents that suppresses expression of Npas2, as described herein. In some embodiments, the “pharmaceutically acceptable composition” and the “pharmaceutical composition” is formulated for topical administration, transdermal administration and/or subcutaneous administration. In an embodiment, the “pharmaceutically acceptable composition” and the “pharmaceutical composition” comprises a pharmaceutically acceptable carrier or excipient.

In one embodiment, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier” or “excipient” includes solvents, dispersion media, coatings, antibacterial and antifungal agents, isotonic and absorption delaying agents, and the like that are physiologically compatible. In one embodiment, the pharmaceutically acceptable carrier is suitable for topical administration, transdermal administration and/or subcutaneous administration. Topical formulations include gels, ointments, creams, lotions, drops and the like.

In an embodiment, the pharmaceutically acceptable carrier is suitable for parenteral administration.

Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, i.e., the agent that suppresses expression of Npas2, use thereof in the pharmaceutical compositions described herein is contemplated. Supplementary active compounds can also be incorporated into the compositions.

Therapeutic pharmaceutical compositions typically are sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols, such as mannitol, sorbitol, or sodium chloride in the composition. Prolonged absorption of injectable compositions can be brought about by including in the composition an agent which delays absorption, for example, monostearate salts and gelatin.

Moreover, an agent that suppresses expression of Npas2, as described herein can be administered in a time release formulation, for example in a composition which includes a slow release polymer. The agent that suppresses expression of Npas2 can be prepared with carriers that will protect it against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating an active compound, such as an agent that suppresses expression of Npas2 described herein, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation include vacuum drying and freeze-drying, which yields a powder of the active ingredient plus any additional desired ingredient from a previously sterile-filtered solution thereof. In some embodiments, an agent that suppresses expression of Npas2, as described herein may be formulated with one or more additional compounds that enhance its solubility.

In one embodiment, a composition of the present invention is administered in a therapeutically effective amount. In one embodiment, a “therapeutically effective amount” is intended to include an amount of an agent or compound of the present invention alone or an amount of the combination of agents or compounds claimed or an amount of an agent or compound of the present invention in combination with other active ingredients effective to act as a suppressor, inhibitor or down-regulator of expression of clock gene, neuronal PAS domain protein 2 (Npas2), effective to improve or accelerate wound healing in a subject, regenerate alveolar bone at a bone loss site of a subject, regenerate connective tissue at a wound site in a subject and/or decrease wound area size of an open wound site of a subject, to which the agent or compound is administered or has been administered. In one embodiment, a “therapeutically effective amount” of an agent that suppresses expression of clock gene Npas2 of the present invention is that amount of agent which is sufficient to provide a beneficial effect to the subject to which the composition is administered.

The following examples are presented in order to more fully illustrate exemplary embodiments of the invention. They should in no way be construed, however, as limiting the broad scope of the invention.

### EXAMPLES

### Example 1

### Npas2-Deficient Fibroblasts Accelerate Skin Wound Healing and Dermal Collagen Reconstruction

### Materials and Methods

### Animal Ethics Statement

The Npas2 knockout (KO) mice (B6.129S6-Npas2tm1Slm/J, Jackson Laboratory, Bar Harbor, Me.) of the C57Bl/6J background were used in this experiment. Npas2 heterozygous mutant (Npas2+/−) mice were generated from cryopreserved sperm samples, and an active breeding colony was established at UCLA. Both Npas2−/− and Npas2+/− mice were used as the experimental groups, and C57Bl/6J wild-type (WT) mice were used as the control group. All of the experimental protocols using animals were reviewed and approved by the UCLA animal research committee (ARC #2003-009) and followed the Public Health Service Policy for the Humane Care and Use of Laboratory Animals and the UCLA Animal Care and Use Training Manual guidelines. All of the animals had free access to food and water and were maintained in regular housing with a 12 hr light/dark cycle at the Division of Laboratory Animal Medicine, UCLA.

### Mouse Dorsal Skin Full-Thickness Excisional Wound Model

The 9- to 14-week-old mice weighing approximately 25 g (WT: four males and four females, Npas2+/−: seven males and Npas2−/−: seven males) were used for the dorsal skin full-thickness wound experiment. After anesthesia with isoflurane inhalation, identical skin wounds were created on the right and left sides of dorsal skin simultaneously by punching a full-thickness skin wound, passing though the panniculus carnosus layer, with a 5 mm dermal biopsy punch (INTEGRA, Integra Life Sciences, Plainsboro, N.J.). These surgeries were performed between 11 a.m. and 1 p. m. Standardized photographs during the course of wound healing were obtained at 0, 2, 4, 6, and 12 days. The skin wound area was measured at each time point (NIH ImageJ ver.1.51). The wound areas on each day were compared by the Kruskal-Wallis test with Dunn's post-test. These mice were sacrificed at 7 days (n=4 in each group) and 14 days (WT: four mice, Npas2+/−: three mice, and Npas2−/−: three mice) for histological analysis. The dorsal skin containing the wound area was dissected as a 1 cm square and immediately fixed with 10% neutral buffered formalin. The sections were stained with hematoxylin and eosin (H-E) for histological evaluation.

### Dermal Fibroblast Cell Culture

Primary fibroblasts from the mouse dorsal skin of each of the three genotypes were cultured using an explant method. The cells were cultured in Dulbecco's modified Eagle's medium (DMEM) with 10% fetal bovine serum and 100 U penicillin/0.1 mg/mL streptomycin at 37_C, 5% CO2 in a humidified incubator. Their genotype was determined by polymerase chain reaction (PCR) targeting WT and mutant Npas2 gene alleles.

### WST-1 Cell Proliferation Assay

The cell proliferation assay was performed using WST-1 reagent (Roche Applied Science, Indianapolis, Ind.). A total of 2,000 cells were seeded into a 96-well reading plate and cultured for the predetermined time points (Days 1, 3, 5, and 7). At each time point, the culture medium was changed to 10% WST-1 regent with medium and incubated for 3 hr (n=4 per time point). The absorption value was read in a spectrophotometer at 450 nm with a plate reader (SYHNERGY H1 plate reader, Biotek, Winooski, Vt.) and compared by two-way analysis of variance (ANOVA), followed by the Tukey test at each time point.

### Circadian Gene Expression in Skin Fibroblasts

Steady-state mRNA expression levels of eight core circadian genes in skin fibroblasts were determined by quantitative real-time PCR (RT-PCR) using Taqman MGB probes (Thermo Fisher Scientific Inc., Waltham, Mass.). Fibroblasts were cultured in 24-well plates and synchronized at 80% to 90% confluency by adding 100 nM dexamethasone to the medium and incubating for 2 hr, followed by washing with DMEM (Nagoshi et al., 2004). Total RNA was extracted using an RNeasy kit (Qiagen, Valencia, Calif.) every 6 hr, starting at 0-48 hr (n=4 per time point) after the synchronization, and their quality and quantity were confirmed by NanoDrop (Thermo Fisher Scientific Inc.). The RT-PCR was performed using commercially available primer/probe mixes (Thermo Fisher Scientific Inc.) as follows: Npas2 (Mm01239312_m1), Bmal1 (=Arnt1: Mm00500223_m1), Clock (Mm00455950_m1), Per1 (Mm0050 1813_m1), Per2 (Mm00478099_m1), Per1 (Mm00478120_m1), Cry1 (Mm00514392_m1), and Cry2 (Mm01331539_m1). Gapdh was used as an internal control. In addition, the LacZ reporter gene expression was determined. The statistical analysis was performed first by two-way ANOVA. The group with the significant interaction P value (P<0.05) by two-way ANOVA and the gene expression at each time point was further subjected to the Tukey test.

### In Vitro Wound Healing Scratch Plate Assay

Fibroblasts were seeded into a 6-well plate and were synchronized as above. After 2 hr, scratch lines were created with a 20 μL plastic pipette and were washed with medium (n=5 per group). These scratched areas were captured by time-lapse photomicrography every hour from 0 to 24 hr. The number of migrated cells into the scratched area was counted at 12 and 24 hr and compared by one-way ANOVA with post hoc Holm test.

### Floating Collagen Gel Contraction Assay

The floating collagen gel contraction assay was performed following the previously established protocol with some modifications (Ngo et al., 2006). A 500 μL aliquot of collagen gel mixture (Collagen Type I, Corning, Manassas, Va.) containing fibroblasts (50,000 cells) was applied to a 24-well plate (n=5 in each group) and placed at room temperature for 20 min. The solidified gels were transferred to a 100 mm diameter dish and cultured (37_C, 5% CO2 in a humidified incubator). The gel images were scanned by a scanner at 0, 6, 12, 24, 48, and 72 hr. The collagen gel area at each time point was measured (NIH ImageJ ver.1.51) and compared by two-way ANOVA, followed by the Tukey test at each time point.

### Single-Cell Contraction Assessment

Single-cell contraction was measured using fluorescently labeled elastomeric contractible surfaces (FLECS) (Forcyte Biotechnologies Inc., Los Angeles, Calif.) (Koziol-White et al., 2016). FLECS plates with the soft silicone elastomer filmed bottom were micropatterned with fluorescent fibrinogen in uniform “X” shapes (70 μm diagonal by 10 μm thick). Approximately 30,000 cells were seeded into a well of 24-well FLECS plate. The plates were placed at room temperature for 40 min and in an incubator (37° C., 5% CO₂) for 30 min for cell attachment. After incubation for initial cell attachment to the X-shape pattern, floating cells were removed by washing with medium, and the plates were incubated for an additional 8 hr. Nuclear staining was performed with Hoechst 33,342 (1:10,000). The images of the fluorescent fibrinogen on the X-shape patterns were captured using a fluorescence microscope with a rhodamine filter. For single-cell contraction evaluation, micropatterns associated with a single nucleus attached at the center of the X shape were selected and categorized to either the no-contract or contract group by comparison with the no-cell pattern. The ratio of contracted patterns per captured image (containing approximately 1,000 X-shape patterns) was compared among each genotype (n=5). The statistical analysis was performed by one-way ANOVA with the post hoc Holm test.

### Gene Expression for Actin, Integrin, and Collagen Subunits

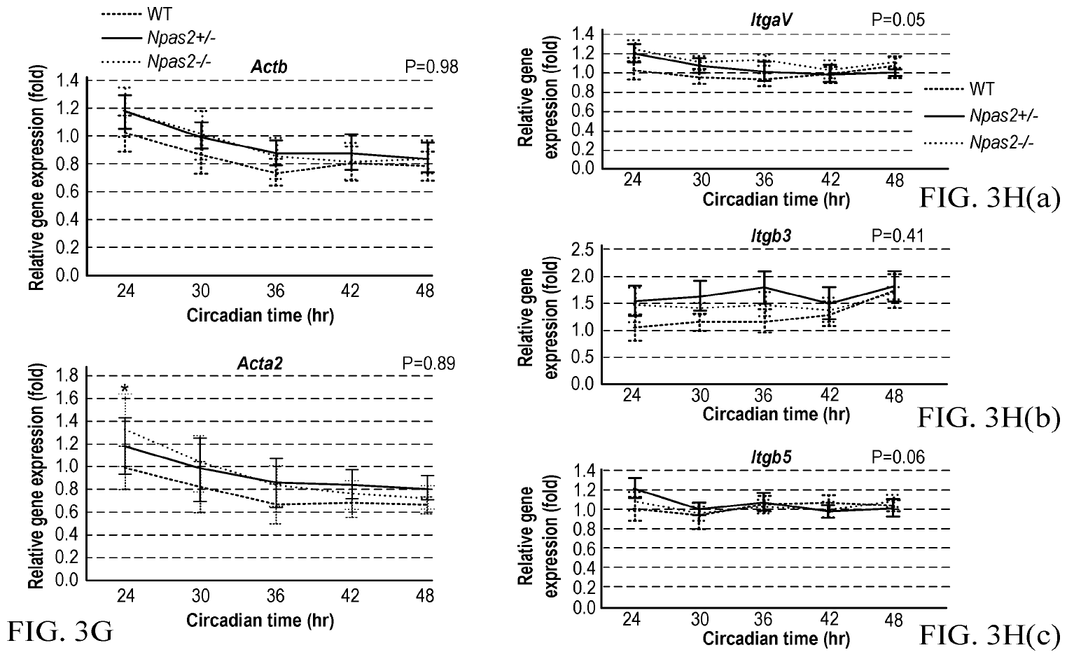

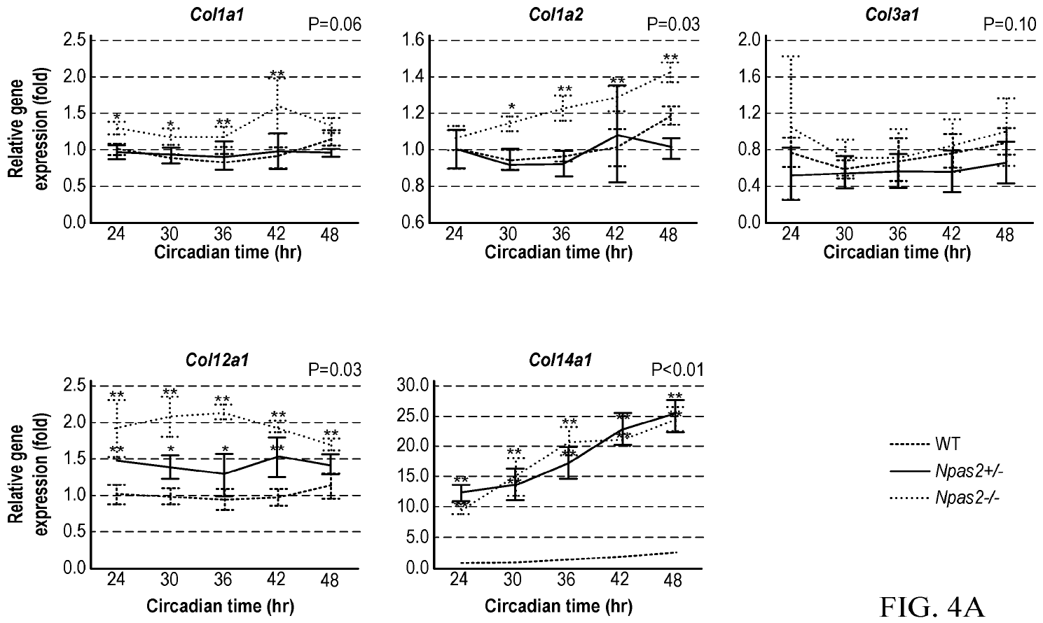

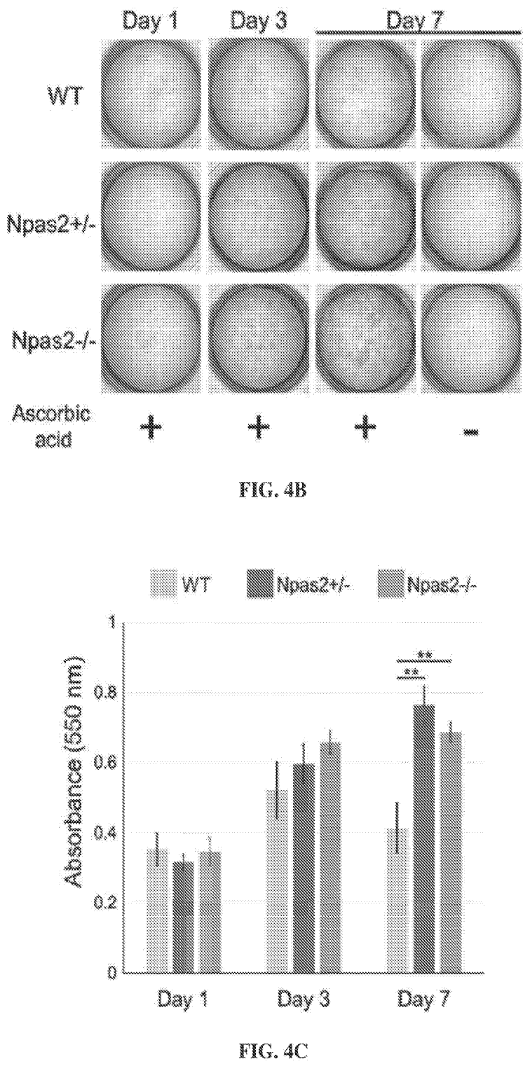

Total RNA samples were extracted from fibroblasts every 6 hr, from 24 to 48 hr after synchronization, as described above. The RNA samples were used for evaluating the gene expression of actin subunits—β-actin (Actb: Mm02619580_g1) and α-smooth muscle actin (α-SMA, Acta2: Mm00725412_s1) (FIG. 3G); integrin subunits—integrin αV (ItgaV: Mm00434486_m1), integrin β3 (Itgb3: Mm00443980_m1), and integrin β5 (Itgb5: Mm00439825_m1) (FIG. 3H); and collagen subunits—type I (Col1a1: Mm00801666_g1 and Col1a2: Mm00483888_m1), type III (Col3a1: Mm00802300_m1), type XII (Col12a1: Mm01148576_m1) and type XIV (Col14a1: Mm008052 69_m1) by Taqman-based qRT-PCR (FIG. 4A). The statistical analysis was performed by two-way ANOVA and Tukey test at each time point.

### Collagen Synthesis Assessment In Vitro by Picrosirius Red Staining

Fibroblasts were seeded into 24-well plates and cultured at 80%-90% confluency in medium supplemented with ascorbic acid (50 μg/mL) for 1, 3, and 7 days. The cells were then fixed with 10% neutral buffered formalin and stained with picrosirius red (PolyScience, Niles, Ill.) for visualizing the collagen. The absorption value was read in a spectrophotometer at 550 nm with a plate reader (SYHNERGY H1 plate reader) and compared by one-way ANOVA with the post hoc Holm test.

### Collagen Fiber Structure at Skin Wound Healing Area by Picrosirius Red Staining and Confocal Laser Scanning Microscopy

The histological sections for the dorsal skin full-thickness wound experiment at 7 and 14 days after surgery were stained with picrosirius red for collagen fibers during wound healing. Collagen fiber structure in the granulation tissue (GT) area, wound closure area (WCA), and intact skin area (ISA) was evaluated using confocal laser scanning microscopy. The distance between the edge of the panniculus carnosus as the original wound width (a) and the distance between the edge of the maturated collagen at the skin punch area, measured as the width of GT (b), were assessed. The ratio of wound closure was calculated by (a−b)/a and was compared by the Kruskal-Wallis test with Dunn's post hoc test.

### Results

### Full-Thickness Dorsal Skin Wound Closure was Accelerated in Npas2−/− Mice

The full-thickness dorsal skin wounds contracted continuously from Days 2 to 12 after surgery, and scar formation was recognized by Day 12 in all genotypes (FIG. 1A). The relative wound area at Day 12 in Npas2−/− mice was significantly smaller than in the other two genotypes (P<0.01) (FIG. 1B). In the histological observation, hyperkeratosis, residual clots, and the immune response in the GT area were observed at 7 days after surgery. Furthermore, the edge of dermis connective tissue with hair follicles moved toward the center of wounds by Day 14. The epithelial layer at the wound area appeared to be similar to the intact skin epithelium, and the immune response had declined in all samples (FIG. 1C). In the present study, mouse dorsal skin full-thickness excisional wounds were generated in the middle of the day (11 a.m.-1 p.m.). It has been reported that skin burn wounds occurring during the night or resting period of humans showed impaired healing, as described by Hoyle et al., 2017 Circadian actin dynamics drive rhythmic fibroblast mobilization during wound healing. Sci Transl Med 9:eaa12774, which is incorporated by reference herein in its entirety. Daytime for the nocturnal mice is equivalent to night for humans. It may be possible that the difference in wound healing between WT and Npas2 KO mice might be more evident if the wound occurred during the dark/active period in mice.

### The Effect of Npas2 KO Mutation on Proliferation and Circadian Rhythm Gene Expression of Skin Fibroblasts

The genotype for each fibroblast sample was determined by PCR. Exon 2 of the mouse Npas2 allele was replaced by the LacZ expression reporter cassette (LacZ/Neo). Because exon 2 encodes the bHLH sequence, the resultant Npas2 molecule lacked the DNA binding function. The amplified PCR product, which was larger than that of WT, recognized Npas2−/− fibroblasts and both the mutant and WT PCR products recognized Npas2+/− fibroblasts.

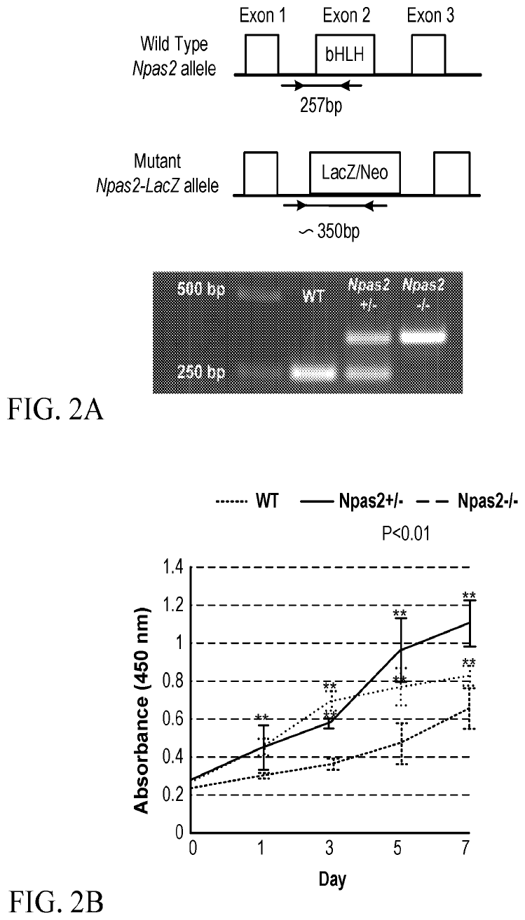

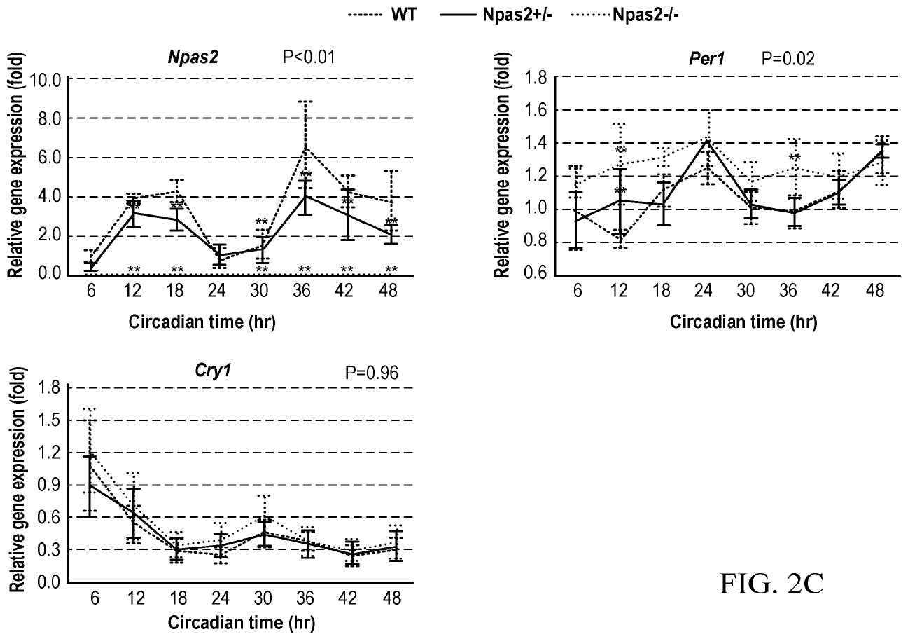

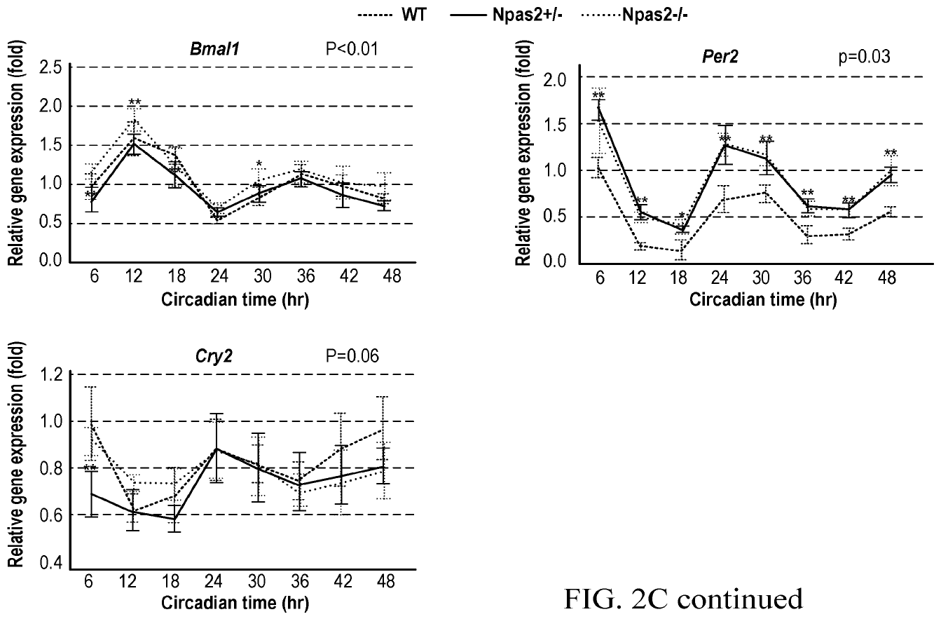

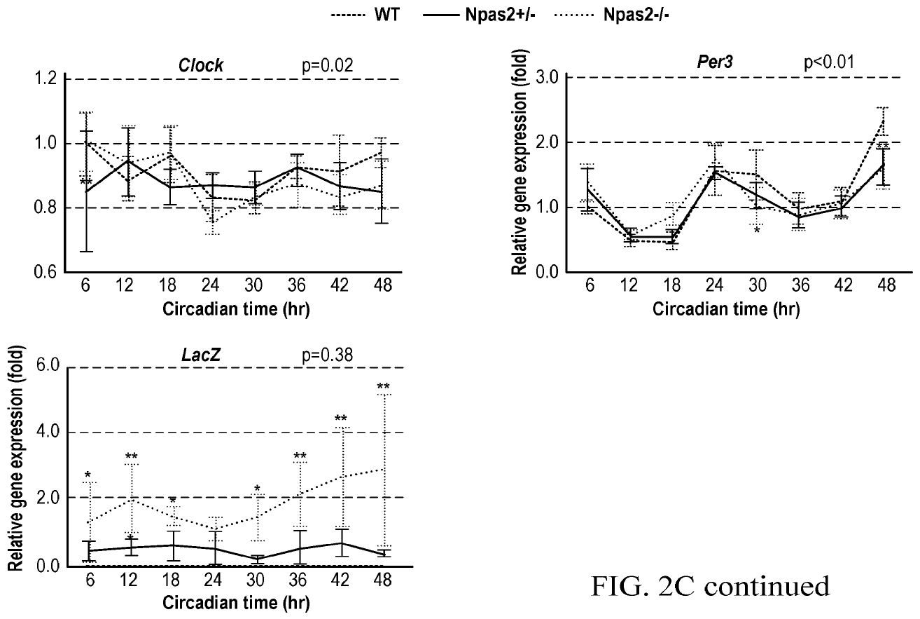

The WST-1 assay indicated that both Npas2+/− and Npas2−/− fibroblasts proliferated faster than WT fibroblasts (P<0.01) (FIG. 2B).

The circadian expression of Npas2 was decreased in Npas2+/− fibroblasts and was undetected in Npas2−/− fibroblasts. However, an effect of the Npas2 KO mutation on the expression patterns of other circadian genes was not observed, except for the Per2 expression (FIG. 2C). The reporter gene (LacZ expression) was detected only in Npas2 KO mice.X

### Accelerated In Vitro Wound Healing of Npas2−/− Skin Fibroblasts by Scratch Test and Floating

The wound healing scratch assay, floating collagen gel contraction assay, and single-cell force assessment with FLECS were performed in vitro. The numbers of migrated Npas2+/− and Npas2−/− fibroblasts were higher than those of WT during 24 hr (Video: https://players.brightcove.net/656326989001/default_default/index.html?videoId=601319767 2001), which was statistically significant (P<0.05) (FIGS. 3A, 3B). However, there was no significant difference between the cell migration rate of Npas2+/− and Npas2−/− fibroblasts (FIGS. 3A, 3B). The floating collagen gel contraction assay showed that Npas2−/− fibroblasts contracted faster than WT and Npas2+/− fibroblasts (P<0.01) (FIGS. 3C, 3D).

### Single-Cell Contraction and Expression of α-SMA and Integrins

The evaluation for single-cell contraction using FLECS (FIG. 3E) revealed that the ratio of contracted Npas2+/− and Npas2−/− fibroblasts was higher than the ratio of WT fibroblasts (P<0.01) (FIG. 3F). The gene expression levels of β-actin (Actb), known to be related with cell migration, and α-SMA (Acta2), known as the factor for upregulating myofibroblast contractile activity, were evaluated by RTPCR (FIG. 3G). The expression of both actin subunits decreased over time. However, there was no significant difference among the three genotypes. The expression of integrin αV (ItgaV), integrin β3 (Itgb3), and integrin β5 (Itgb5) did not show any circadian rhythm in dermal fibroblasts. Npas2 KO mutation did not affect the steady-state level of the examined integrin subunits (FIG. 3H).

Npas2−/− Fibroblasts Increased Dermis-Like Collagen Synthesis In Vitro

The gene expression levels of collagen subunits type I (Col1a1, Col1a2), type III (Col3a1), type XII (Col12a1), and type XIV (Col14a1) were investigated in this experiment (FIG. 4A). Overall, no circadian pattern was observed in these collagen mRNAs. Col1a1 and Col1a2 in Npas2−/− fibroblast were more highly expressed than in WT and Npas2+/− fibroblasts; however, the interaction P value was significant only for Col1a2. No difference was observed for the Col3a1 expression. There was an increase of Col12a1 expression in Npas2+/− and Npas2−/− fibroblasts. Strikingly, a significantly elevated expression of Col14a1 was found in both Npas2+/− and Npas2−/− fibroblasts compared to WT fibroblasts. The picrosirius red staining for fibroblasts cultured with ascorbic acid supplementation showed a strong, positive reaction, indicating collagen fiber formation and accumulation in Npas2+/− and Npas2−/− fibroblasts (FIG. 4B); their absorbance at 550 nm was significantly higher than that in WT fibroblasts at Day 7 (P<0.01) (FIG. 4C).

Dermis-Like Collagen Fiber Reconstruction during Skin Wound Healing of Npas2−/− Mice

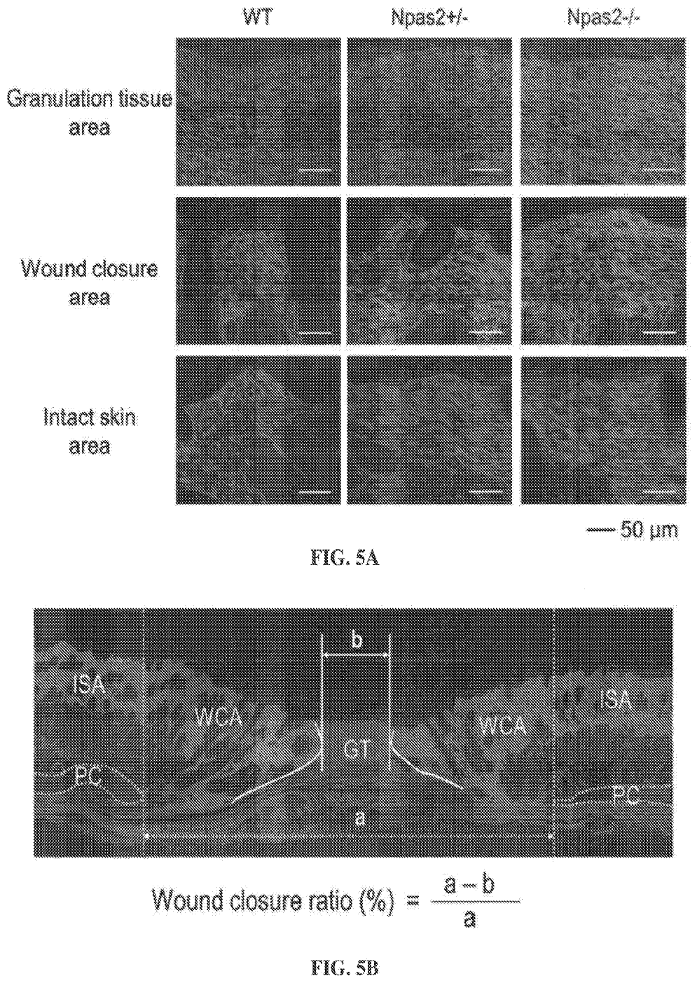

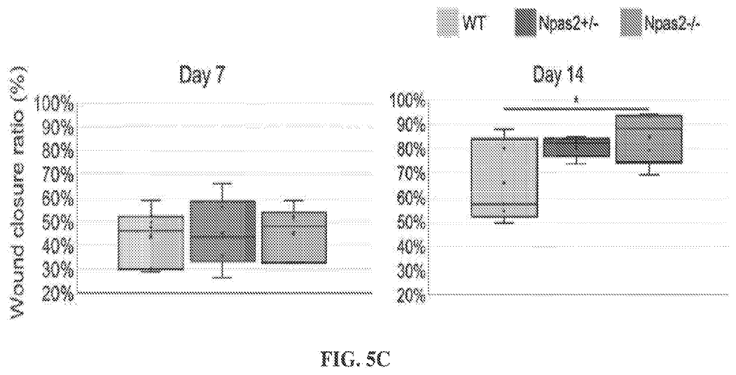

Histological sections of the full-thickness skin wound area with picrosirius red staining were examined by confocal laser scanning microscopy (FIG. 5A). There was no obvious difference in collagen fiber structures in the ISA; however, collagen fibers in both the GT area and the WCA appeared to be thicker in Npas2+/− and Npas2−/− samples than in those of WT. In particular, collagen fibers of GT in Npas2−/− samples appeared more organized, partially resembling the intact skin collagen structure. The histological measurement of wound closure was performed with picrosirius red-stained sections (FIG. 5B). The ratio of wound closure of Npas2+/− and Npas2−/− samples was greater than that of WT, although statistical significance was achieved only between WT and Npas2−/− samples at Day 14 (P<0.01) (FIG. 5C).

### Discussion

Mammalian skin is a large barrier tissue composed of the epithelial layer (epidermis) and underlining connective tissue (dermis). This study proposes a novel role of the circadian clock in dermal fibroblasts for skin wound healing, which may possibly enable dermal connective tissue collagen reconstruction. Once injured, the skin epithelial cells actively proliferate and migrate over the wound, leading to the rapid establishment of a barrier layer. By contrast, dermal fibroblasts are slow in proliferation and migration into the wound area. Furthermore, wound fibroblasts do not maintain the dermal fibroblast phenotype, but acquire a new phenotype, in part, contributing to the formation of GT and scarring. The present study demonstrated the accelerated healing of the well-established skin full-thickness wound model, as described by Kowalska et al., 2013, NONO couples the circadian clock to the cell cycle. Proc Natl Acad Sci USA 110:1592-1599, which is incorporated by reference herein in its entirety, in Npas2+/− and Npas2−/− mice, potentially through faster wound closure and/or smaller scarring than that of WT mice (FIG. 1). As such, this study focused on the role of Npas2 KO mutation on the behavior of dermal fibroblasts as a mechanistic investigation.

Npas2 is a core circadian rhythm gene encoding a basic HLH transcription factor and is highly expressed in skin fibroblasts. Npas2 has been postulated to compensate the role of Clock, whose expression rate in fibroblasts was comparatively low (FIG. 2C). In the case of retinal cells, knock down of the Clock gene reduced mRNA and protein levels of Npas2, whereas knock down of Npas2 did not affect either the mRNA or protein levels of Clock. The data herein corroborated the previous observation that Npas2 KO mutation did not significantly affect the expression of the core circadian rhythm genes (FIG. 2C). Thus, the effect of Npas2 KO mutation may be mediated by mechanisms other than the disruption of the circadian rhythm. The expression of Npas2 in the SCN peaks at the dark/active period in mice. Wound responses in mice would be expected to show a daily rhythm. However, this issue was not explored in the present study.

Three-dimensional collagen gels containing fibroblasts have been used to model tissue remodeling, wound contraction, and fibrosis. The primary mechanism of fibroblast-embedded gel contraction in vitro is due to fibroblast locomotion forces. The cell traction force is applied to the substrate ECM, contributing to the collagen gel contraction. The accelerated collagen gel contraction was demonstrated by Npas2+/− and Npas2−/− fibroblasts (FIGS. 3C, 3D), suggesting the increased fibroblast locomotion forces. It was reported that silencing the NPAS2 expression in human colorectal cancer cells accelerated cell migration. The present study also showed accelerated migration by Npas2+/− and Npas2−/− fibroblasts in an in vitro scratch test (Video: https://players.brightcove.net/656326989001/default_default/index.html?videoId=601319767 2001; FIGS. 3A, 3B). The activation of extracellular signal-regulated kinase (ERK) and phosphoinositide-3 kinase/protein kinase B (PI3K/AKT) through phosphorylation is well known to regulate cell migration, collagen gel contraction, and skin wound healing. The activation of these signaling pathways was suggested in the phenotype conversion of fibroblasts toward myofibroblasts, such as an increased expression of α-SMA. In the present study, the phenotype conversion of fibroblasts was not suggested by the Npas2 KO mutation (FIG. 3G), and thus, the involvement of a myofibroblast-like phenotype in the modulated collagen gel contraction and fibroblast migration was ruled out. However, it is important to characterize the effect of Npas2 KO mutation on phosphorylation in the ERK/Akt/FAK pathway.

During migration, fibroblasts adhere to the extracellular matrix (ECM) through integrin molecules and generate a single-cell traction force. A recently developed single-cell contraction assay that required cell adhesion to the FLECS printed with fibronectin was used, as described by Koziol-White et al., 2016 Inhibition of PI3K promotes dilation of human small airways in a rho kinase-dependent manner Br J Pharmacol 173:2726-2738; Pushkarsky et al., 2018 Elastomeric sensor surfaces for high-throughput single-cell force cytometry. Nat Biomed Eng 2:124-137, each of which is incorporated by reference herein in its entirety. The FLECS assay showed that mouse dermal fibroblasts increased the cell contraction behavior by Npas2 KO mutation (FIG. 3F). Wound-induced transformation of fibroblasts to myofibroblasts has been postulated to play a pathological role in tissue contraction and fibrosis formation. Separately, the increased expression of alpha and beta integrins mediating cell adhesion to fibronectin were thought to be critical for cell contractility driven wound fibrosis formation. For example, the significantly elevated expression of integrin αVβ3 has been postulated to cause idiopathic pulmonary fibrosis. In the present study, the steady-state expression of myofibroblast marker α-SMA as well as integrin subunits αV, β3, and β5 was not altered by Npas2 KO mutation in dermal fibroblasts (FIGS. 3G, 3H). Therefore, the increased fibroblast contractility by Npas2 KO mutation may not result in the abnormal wound healing phenotypes of pathological wound contraction or fibrosis formation.

Connective tissue ECM molecules, in particular the FACIT class of collagens, have been shown to influence cell migration and cell contraction through integrin-mediated cell adhesion. The FACIT class of collagens has been postulated to decorate the surface of collagen fibers. The externally exposed N-terminal globular domains, such as NC3 of type XII and type XIV collagens, have been shown to be essential in fibroblast-mediated collagen gel contraction. Thus, the inventors postulate that the increased expression of type XII and XIV collagens in Npas2 KO fibroblasts might affect the migration and gel contraction behaviors.

It has been reported that downregulation of Npas2 expression is related to cell cycle progression and DNA repair capacity, although there are conflicting reports on the effect of Npas2 modulation on cell proliferation. The present study indicated that Npas2 KO mutation increased fibroblast proliferation (FIG. 2B), which may have confounded the cell migration assay. When the time-lapse microscopy was evaluated (Video: https://players.brightcove.net/656326989001/default_default/index.html?videoId=6013197672001), no proliferating cells were observed within the scratch area for all genotypes, suggesting that the effect of Npas2 on cell proliferation and migration occurs through mechanisms other than cell proliferation.

Previously, it was reported that titanium-based biomaterials increased the Npas2 expression of bone marrow mesenchymal stromal cells (BMSC) concomitantly with an elevated expression of cartilage collagens types II, IX, and X, suggesting that Npas2 might mediate biomaterial-induced BMSC differentiation. Thus, the first step of mechanistic dissection was to examine skin fibroblast differentiation through dermal-related collagens. Skin dermal collagen ECM is primarily composed of fibril-forming type I and type III collagens, which form thick collagen fibers. FACIT collagen types XII and XIV have been found in developing skin. Type XII and XIV collagens are postulated to decorate the surface of collagen fibers and regulate the physiological ECM organization with tissue-specific functions. By contrast, wound fibroblasts abundantly synthesize collagen ECM with different properties in the GT. The present study revealed a striking upregulation of FACIT collagen types XII and XIV by Npas2 KO fibroblasts (FIG. 4A). The in vitro collagen fiber formation depicted by picrosirius red staining showed thick collagen fibers in the cultures of Npas2+/− and Npas2−/− fibroblasts (FIGS. 4B, 4C). The robustly increased FACIT expression might contribute to the re-organization of dermis-like collagen fibers in the skin wound. In fact, Npas2−/− mice demonstrated an increased WCA containing mature dermis-like collagen structure (FIG. 5A). Furthermore, the GT of Npas2−/− mice showed thicker collagen fibers, in part, resembling the dermis-like collagen fiber structure. Taken together, the inventors propose that fibroblasts with decreased Npas2 expression may differentiate to dermal fibroblasts, not myofibroblasts or GT fibroblasts, and Npas2-suppressed fibroblasts might induce their ability to better reconstruct, if not partially regenerate, the dermal collagen architecture.

The present Example demonstrates that Npas2 suppression in peripheral skin fibroblasts modified cell behaviors and was depicted by accelerated cell proliferation, cell migration, and cell contraction forces in vitro. Moreover, Npas2 suppression resulted in increased dermis FACIT collagen synthesis and the formation of thick collagen fibers. These fibroblastic phenotypes appeared to have contributed to better skin wound healing and the potential reconstruction of dermis collagen architecture. Within the scope of this study, the mechanism of circadian clock molecules, such as Npas2, in dermal wound healing may facilitate skin-specific cell differentiation. From these results, the inventors propose that Npas2 may be an attractive therapeutic target for improving skin wound healing.

### Example 2

### Screening for Small Molecule Compounds Suppressing Npas2 Expression Using High Throughput Screening

In Example 1, mouse dermal fibroblasts carrying Npas2-LacZ reporter genes that had been developed and validated, confirmed that the detection of LacZ was consistent with Npas2 expression, as described by Sasaki H, et al., Neuronal PAS Domain 2 (Npas2)-Deficient Fibroblasts Accelerate Skin Wound Healing and Dermal Collagen Reconstruction. Anat Rec (Hoboken). 2019, which is incorporated by reference herein in its entirety.

UCLA academic laboratory established the research platform for regenerative chronotherapy targeting Npas2: (1) high throughput screening (HTS) of small compounds; (2) in vitro biological validation; and (3) in vivo testing in mouse periodontitis model. This research platform successfully identified a compound, Reserpine, possessing the regenerative activity, as well as additional compounds suppressing Npas2.

The inventors found that HTS was less effective in identifying compounds that decreased the target gene expression. A number of compounds suppressing Npas2 turned out to be false positives due to cytotoxicity leading to cell death or growth suppression.

The HTS protocol was improved to better identify Npas2 suppressors. Mouse MSC carrying Npas2-LacZ reporter gene, as described by Hassan N, et al., PLoS One. 2017; 12(8):e0183359, which is incorporated by reference herein in its entirety, was immortalized using SV40 in order to accommodate a large-scale HTS. The expression of LacZ reporter gene of immortalized and primary MSC was consistent (data not shown). This cell system was used to screen FDA Approved Drug Library (1120 compounds). The same library was also screened for cell viability by Calcein AM Hoechst 33342 staining Cell migration assays using the Oris™ Pro Cell Migration Assay plate, as described by Joy M E et al., PloS one. 2014; 9(2):e88350, which is incorporated by reference herein in its entirety, was used for a high-throughput migration assay using human skin fibroblasts

The combined Z score was used to identify hit compounds (Table 1). A small number of compounds suppressing Npas2 were identified (Table 1). The highest hit for Npas2 expression suppressor compound was Dwn1, i.e., Reserpine. (Npas2 suppression z score: −2.57; and cell migration z score: 4.19).

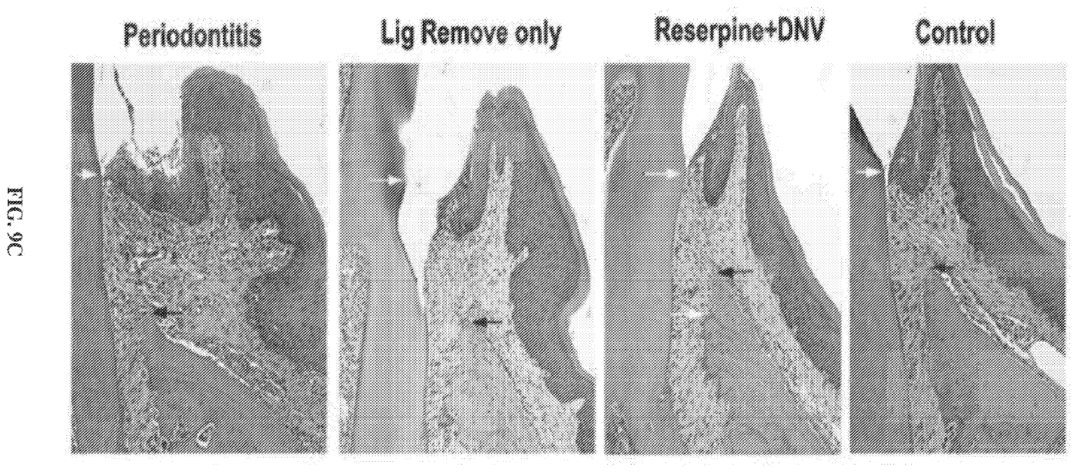

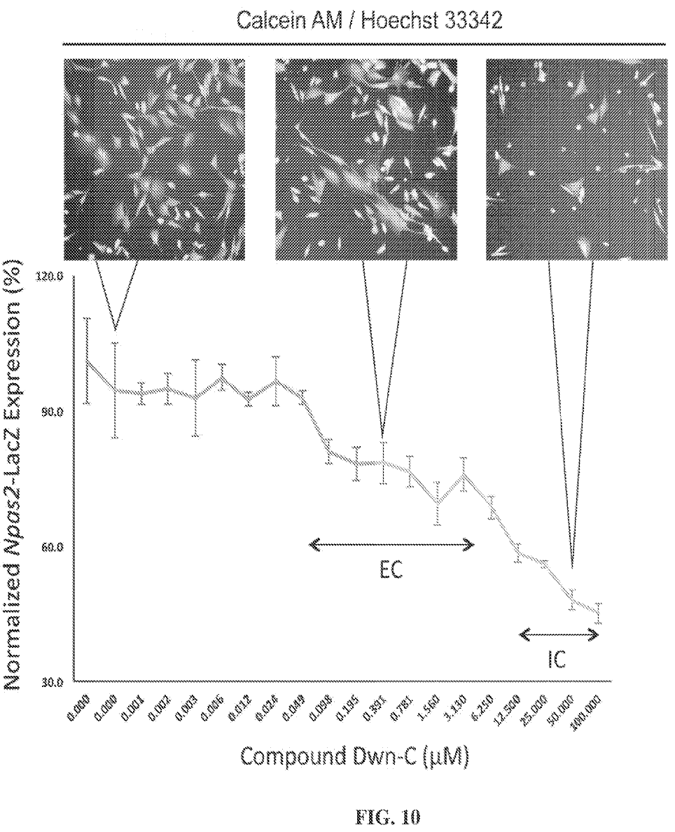

Dwn1 was identical to the previously identified DwnC compound from the LOPAC library. Dwn1 compound (Reserpine) was selected for serial dilution analysis. The effective concentration (EC) was determined in a range of 0.5-10 μM and the inhibitory concentration (IC) was at >12.5 μM (FIG. 10). Dwn1 was used for preliminary in vitro and in vivo studies described below. Additional compounds suppressing Npas2 were identified (Table 2).

TABLE 1
Small Molecule Compounds Suppressing
Npas2 Expression Identified by HTS of FDA
Drug Library. MED is a medicinal compound.

| | Migration | | | | |
|---|---|---|---|---|---|
| | (standard- | | | | |
| | ized | NPAS | | | |
| | viability) | Z × | | | |
| NPAS2 | Z | Via- | Molecule | | |
| Z Score | Score | bilityZ | Name | Action | Activity |
| −2.57 | 4.19 | −10.77 | Reserpine | Inhibitor | anti- |
| | | | | | hyper- |
| | | | | | tensive, |
| | | | | | MED |
| −2.72 | 2.13 | −5.79 | Antimycin | | MED |
| | | | A | | |
| −1.99 | 1.79 | −3.56 | Niflumic | Inhibitor | Misc. |
| | | | Acid | | Channels, |
| | | | | | analgesic, |
| | | | | | anti- |
| | | | | | inflam- |
| | | | | | matory, |
| | | | | | MED |
| −2.1 | 1.22 | −2.56 | Molindone | An- | MED |
| | | | hydro- | tagonist | |
| | | | chloride | | |
| −2.06 | 1.12 | −2.31 | Mefex- | | stimulant |
| | | | amide | | (central), |
| | | | hydro- | | MED |
| | | | chloride | | |

TABLE 2
Additional Npas2 Expression Suppressors

| | Migration | | | | |
|---|---|---|---|---|---|
| | (standard- | | | | |
| NPAS2 | ized | | | | |
| Z | viability) Z | Molecule | | | NpasZ × |
| Score | Score | Name | Action | Activity | ViabilityZ |
| −2.41 | 0.805 | Econazole | | antifungal, | −1.94005 |
| | | nitrate | | MED | |
| −2.16 | 0.666 | Aceclofenac | | MED | −1.43856 |
| −2.16 | 0.64 | Pravastatin | | | −1.3824 |
| −2.39 | 0.572 | Tyloxapol | | MED | −1.36708 |
| −2.01 | 0.647 | Isosorbide | | | −1.30047 |
| | | mononitrate | | | |
| −2.08 | 0.577 | MS-1500387 | | anti- | −1.20016 |
| | | | | neoplastic, | |
| | | | | purine | |
| | | | | anti- | |
| | | | | metabolite | |
| −2 | 0.429 | (S)-(-)- | | MED | −0.858 |
| | | Atenolol | | | |
| −2.02 | 0.239 | Butenafine | | | −0.48278 |
| | | Hydrochloride | | | |
| −2.2 | 0.198 | Aceclidine | | | −0.4356 |
| | | Hydrochloride | | | |
| −2.04 | 0.181 | Atropine sulfate | | MED | −0.36924 |
| | | monohydrate | | | |
| −2.74 | −0.105 | Trimethadione | | MED | 0.2877 |
| −2.36 | −0.157 | Chlorphensin | | MED | 0.37052 |
| | | carbamate | | | |
| −2.8 | −0.188 | Mafenide | | antibacterial, | 0.5264 |
| | | hydrochloride | | MED | |
| −2.41 | −0.243 | Nifenazone | | analgesic, | 0.58563 |
| | | | | anti- | |
| | | | | inflam- | |
| | | | | matory, | |
| | | | | MED | |
| −2.02 | −0.466 | Articaine | | MED | 0.94132 |
| | | hydrochloride | | | |
| −2.04 | −0.6 | Theobromine | An- | diuretic, | 1.224 |
| | | | tagonist | broncho- | |
| | | | | dilator, | |
| | | | | cardiotonic, | |
| | | | | ALK | |
| −3.43 | −0.402 | Nifuroxazide | | MED | 1.37886 |
| −2.6 | −0.749 | SAM001246626 | | | 1.9474 |
| −2.27 | −1.21 | Dropropizine | | antitussive, | 2.7467 |
| | | (R,S) | | MED | |
| −2.36 | −2.08 | Diethyl- | | anthelmintic, | 4.9088 |
| | | carbamazine | | MED | |
| | | citrate | | | |
| −3.22 | −1.53 | MS-1501214 | | ACE | 4.9266 |
| | | | | inhibitor, | |
| | | | | anti- | |
| | | | | hypertensive | |
| −2.64 | −1.91 | Dolasetron | | | 5.0424 |
| | | mesilate | | | |
| −2.01 | −2.56 | Estrone | | MED | 5.1456 |
| −2.42 | −2.31 | Prednisolone | | MED | 5.5902 |
| −2.45 | −4.56 | Daunorubicin | | MED | 11.172 |
| | | hydrochloride | | | |
| −3.29 | −4.91 | Cycloheximide | | protein | 16.1539 |
| | | | | synthesis | |
| | | | | inhibitor, | |
| | | | | MED | |
| −4.34 | −8.13 | Monensin | | MED | 35.2842 |
| | | sodium salt | | | |

### Example 3

### In Vitro Phenotype Study

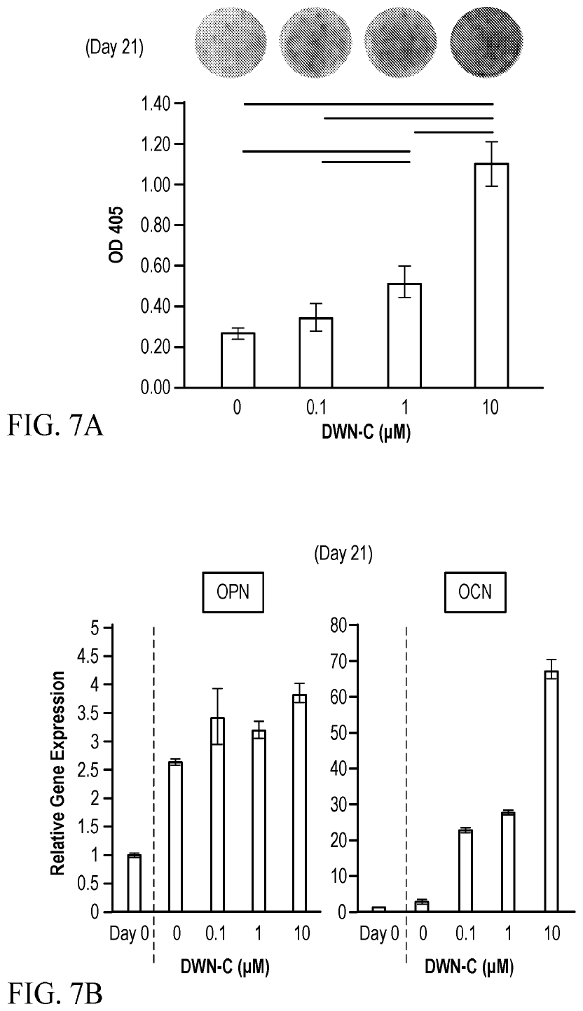

Reserpine was selected and its effect on bone marrow stromal cells (BMSC) was characterized for osteogenic differentiation. Reserpine dose-dependently accelerated in vitro mineralization (FIG. 7A). The expression of late-stage osteogenic differentiation marker: osteoclacin (Ocn) was significantly increased at day 21, whereas osteopontin (Opn) was mildly affected (FIG. 7B).

To evaluate the mechanism of action involving Npas2, Reserpine was applied to WT, Npas2+/− and Npas2−/− mouse BMSC and evaluated for in vitro osteogenic differentiation. The effect of Reserpine was reproducibly observed in WT as well as Npas2+/− BMSC. However, it did not modulate Npas2−/− BMSC, suggesting that the mode of action (MOA) of Reserpine in supporting osteogenic differentiation of BMSC was suppressing Npas2.

### Example 4

### In Vivo Skin Wound Healing Study

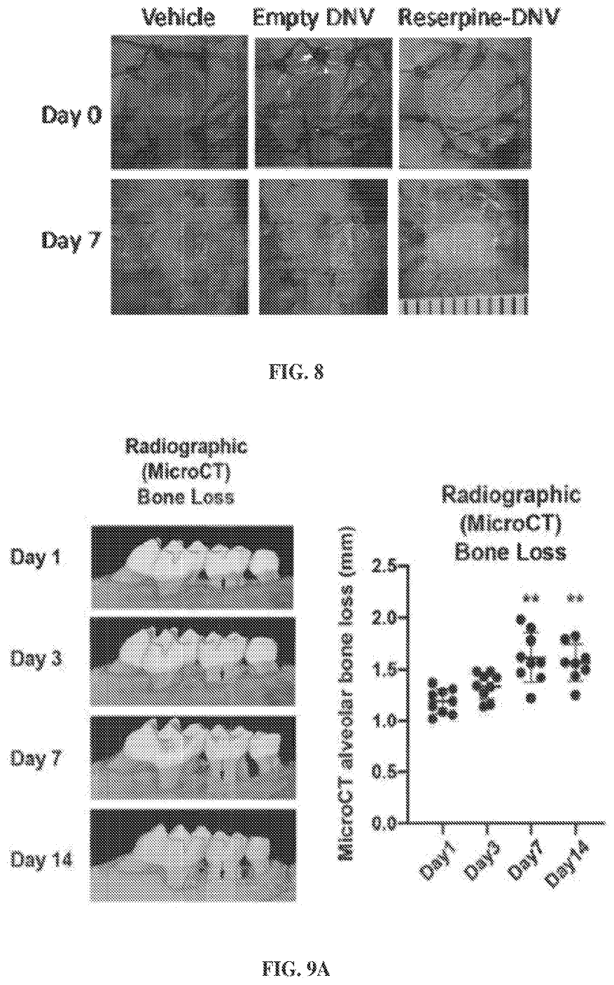

An in vivo application study was conducted to evaluate the dermal wound healing capability of Npas2 suppression using Reserpine. A trans-dermal drug using deformable nanoscale vesicle (DNV) encapsulating Reserpine has been prototyped. It has been demonstrated, as described by Subbiah N, et al., Deformable Nanovesicles Synthesized through an Adaptable Microfluidic Platform for Enhanced Localized Transdermal Drug Delivery. J Drug Deliv. 2017; 2017:4759839, which is incorporated by reference herein in its entirety, that DNVs can penetrate the keratinized epidermis of mouse skin. Mouse dorsal skin punch wounds (5 min in diameter) were created and fixed with a custom-made ring, then vehicle (MilliQ water), empty DNV or Reserpine encapsulated DNV were applied on the wounds every day. On day 7, wounds treated with Reserpine encapsulated DNV showed accelerated wound healing compared with other applications (FIG. 8).

### Example 5

### Periodontal Tissue Regeneration in the Mouse Periodontitis Model

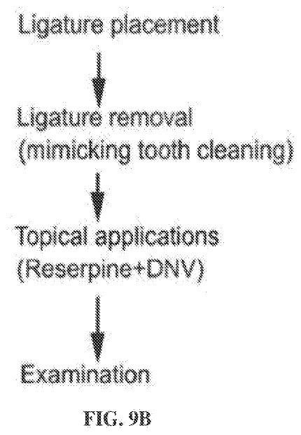

A commonly used mouse model of ligature-induced periodontitis was utilized in investigations on pathological mechanisms. The ligature was placed around the maxillary second molar at Day 0. Periodontitis-induced alveolar bone resorption was observed (FIG. 9A) and gingival connective tissue degeneration was observed (FIG. 9C). The ligature placement induced severe inflammation, epithelial hyperplasia and connective tissue collagen disarrangement consistent with periodontitis (FIG. 9C). Then the ligature was removed at Day 7 and allowed to heal for 1 week. This process mimicked the routine dental treatment of scaling. Removal of the ligature significantly decreased the inflammatory reaction (inflammation); however, epithelial and connective tissue abnormalities remained and alveolar bone did not regenerate (FIG. 9C). In this model, after ligature removal, Reserpine incorporated in DNV (hereafter “Reserpine+DNV”) was topically applied on mouse gingival tissue (FIG. 9B). The Reserpine+DNV treated group demonstrated the re-arrangement of gingival connective tissue and collagen, similar to control. The regeneration of alveolar bone also was observed (FIG. 9C).

### Example 6

### Efficacy of Npas2 Suppressing Compounds for Accelerated Osteogenic Differentiation In Vitro

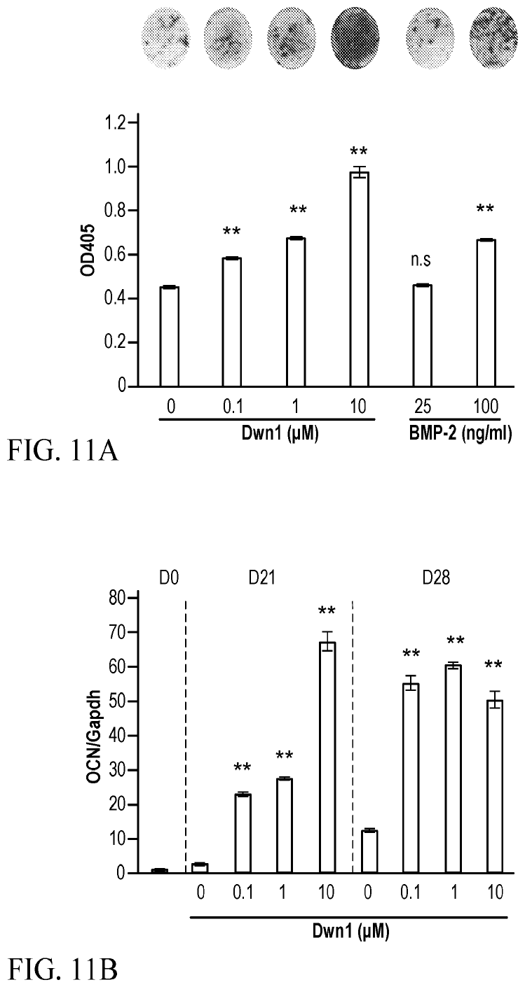

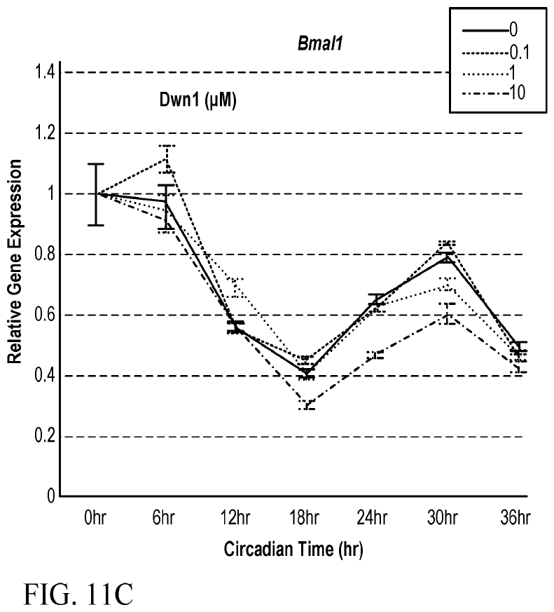

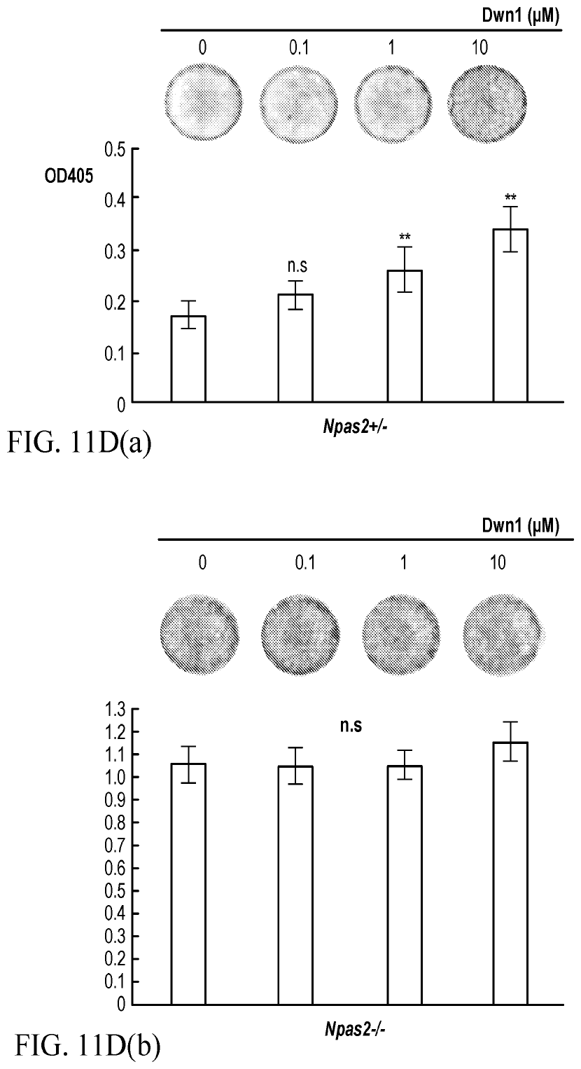

The small molecule compound that scored the best combined z score in Example 2, Dwn1 (Reserpine) was selected for in vitro biological assays. The osteogenic induction medium supplemented with Dwn1 dose dependently increased the in vitro mineralization of MSC (FIG. 11A). BMP-2 has been used as a gold standard for accelerated osteogenic differentiation of MSC (48, 49). The effect of Dwn1 (1 μM) was found equivalent to BMP-2 (100 ng/ml) supplementation (FIG. 11A). The osteogenic differentation by Dwn1 was also supported by the accelerated expression of osteocalcin (OCN) (FIG. 11B). The expression of core clock gene Bmal1 was not affected by Dwn1 (FIG. 11C). Dwn1 increased in vitro mineralization of Npas2+/− MSC, but not of Npas2−/− MSC (FIG. 11D), implying that the effect of Dwn1 (Reserpine) was facilitated primarily by Npas2 suppression.

### Example 7

### Rapid Bone Regeneration in Tooth Extraction Socket of Npas2 KO Mice

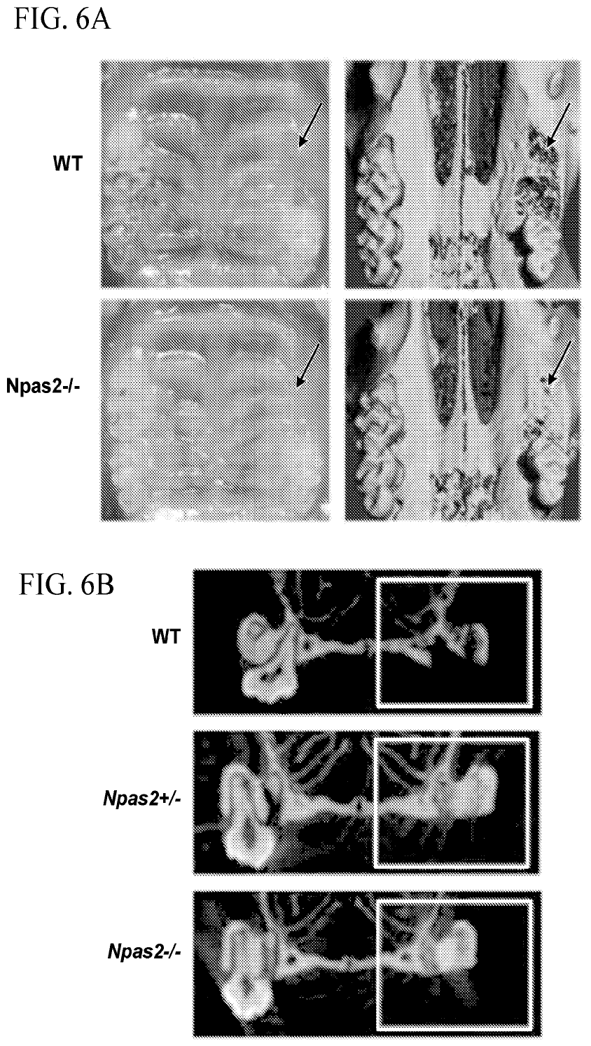

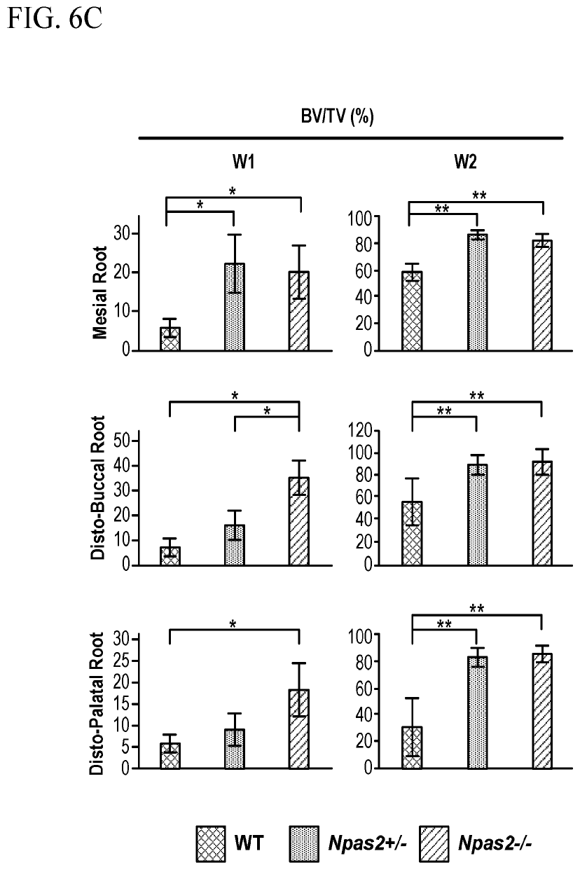

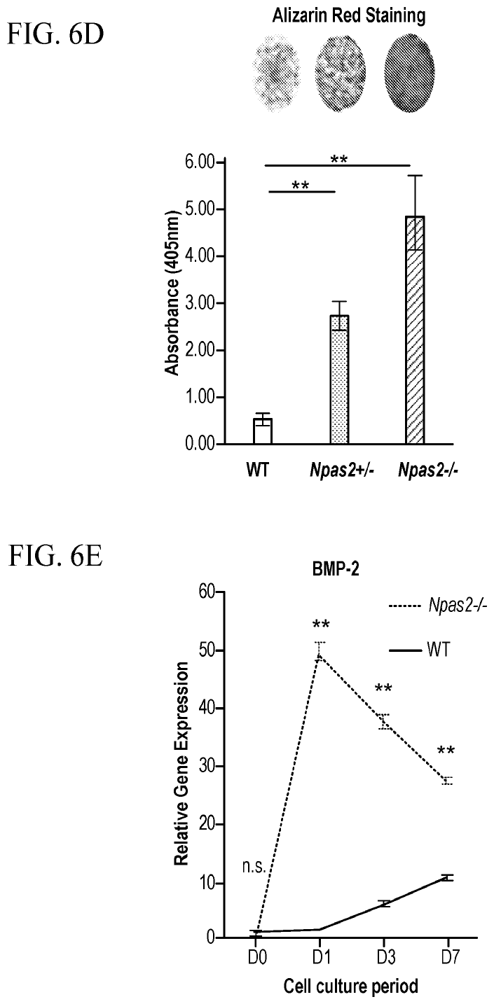

C57B16J (B6) wild type (WT) mice were treated with maxillary left first molar extraction, which underwent wound healing of both oral mucosa and alveolar bone (white arrow). A rapid bone regeneration in tooth extraction socket of Npas2 KO mice was observed (FIG. 6A-6C). It has been previously demonstrated that bone formation in the extraction socket undergoes intramembranous ossification without cartilage precursor tissue. In wild type mice, the bone formation was limited to the bottom half of extraction sockets. By contrast, Npas2 KO mice demonstrated over 80%˜100% of the socket filled with new bone. The “bone formation” in the top half of extraction socket requires regenerative agents such as BMP-2, as described by Coomes A M, et al., Buccal bone formation after flapless extraction: a randomized, controlled clinical trial comparing recombinant human bone morphogenetic protein 2/absorbable collagen carrier and collagen sponge alone. J Periodontal. 2014; 85(4):525-35, which is incorporated by reference herein in its entirety. Because the top half of extraction socket is not normally filled by new bone, the “bone formation” induced by regenerative agents such as BMP-2 at the top half of extraction socket was considered de novo bone formation or bone regeneration, Therefore, the bone filling of the entire extraction socket of Npas2 KO mice can be interpreted as an unprecedented bone regeneration activity. Bone marrow derived mesenchymal stromal/stem cells (MSC, also called BMSC) of Npas2 KO mice also demonstrated the robust in vitro mineralization (FIG. 6D) and increased BMP-2 expression when exposed to osteogenic medium (FIG. 6E), suggesting that circadian rhythm, in particular Npas2, may regulate bone regeneration. Npas2 KO MSC also showed increased expression of BMP receptors (data not shown). These observations have provided the rationale to explore the novel opportunity for therapeutic development.

The inventors hypothesize that the therapeutic suppression of Npas2 increases the capacity of maintenance of unmodified function and phenotype of connective tissues, such as dermal fibroblasts and alveolar bone regeneration, and thus, suppression of Npas2 raises the capacity of connective tissue regeneration and alveolar bone regeneration.

### Example 8

### Efficacy of Npas2 Suppressing Compounds for Alveolar Bone Regeneration in Mouse Ligature-Induced Periodontitis Model

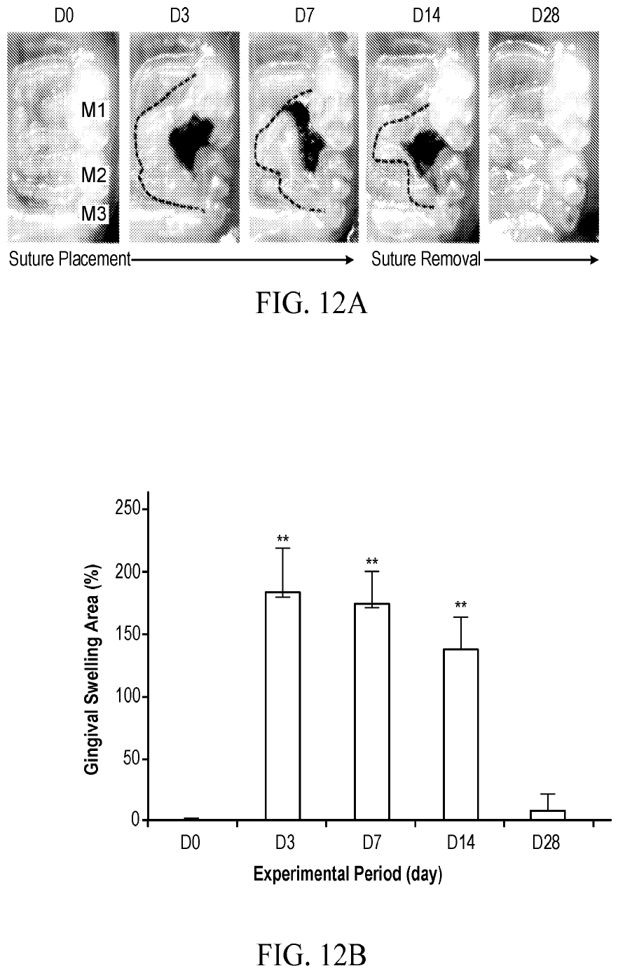

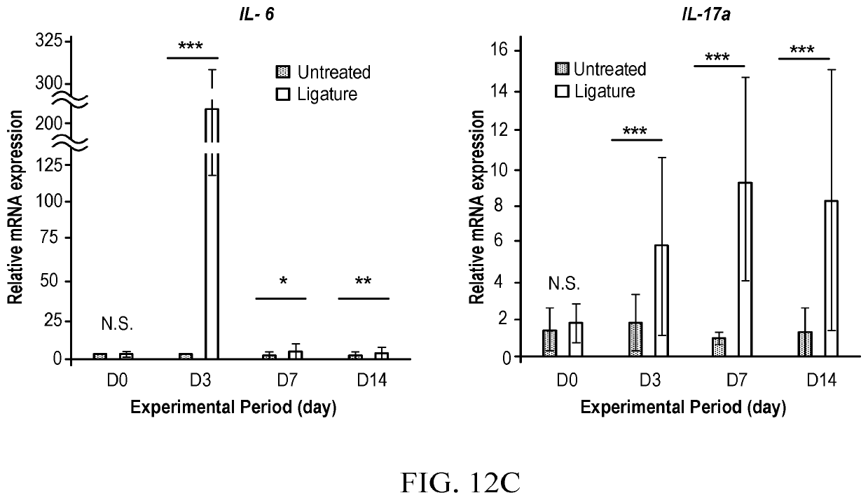

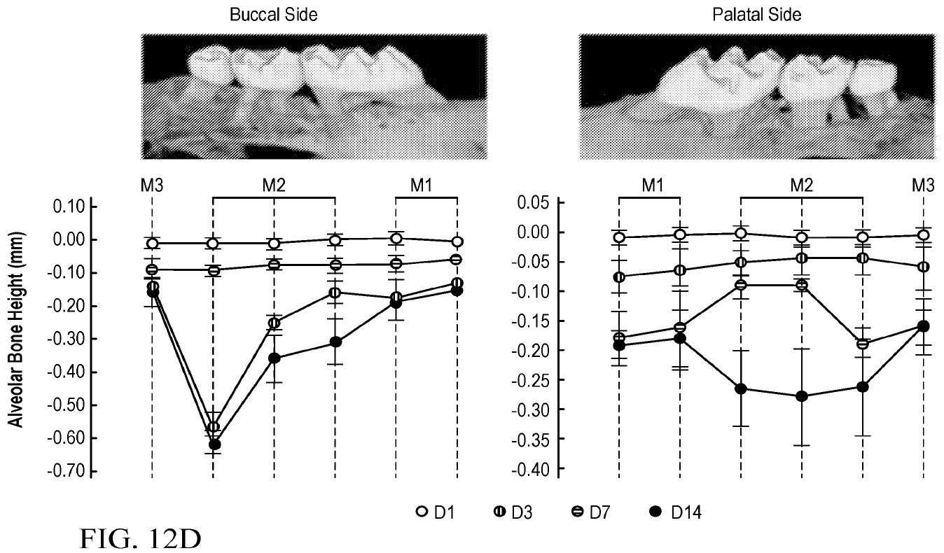

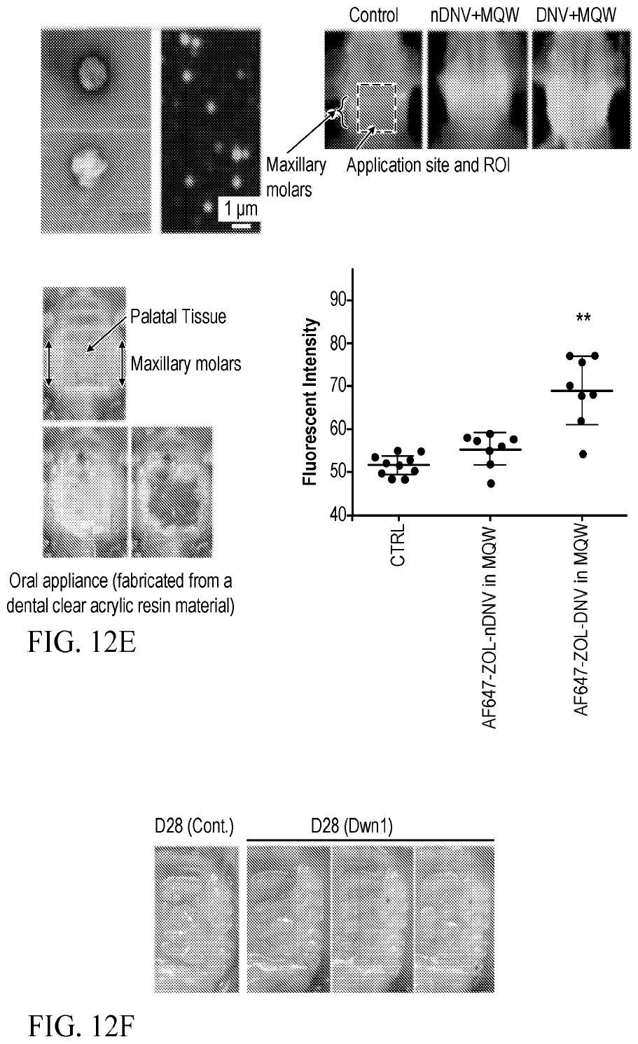

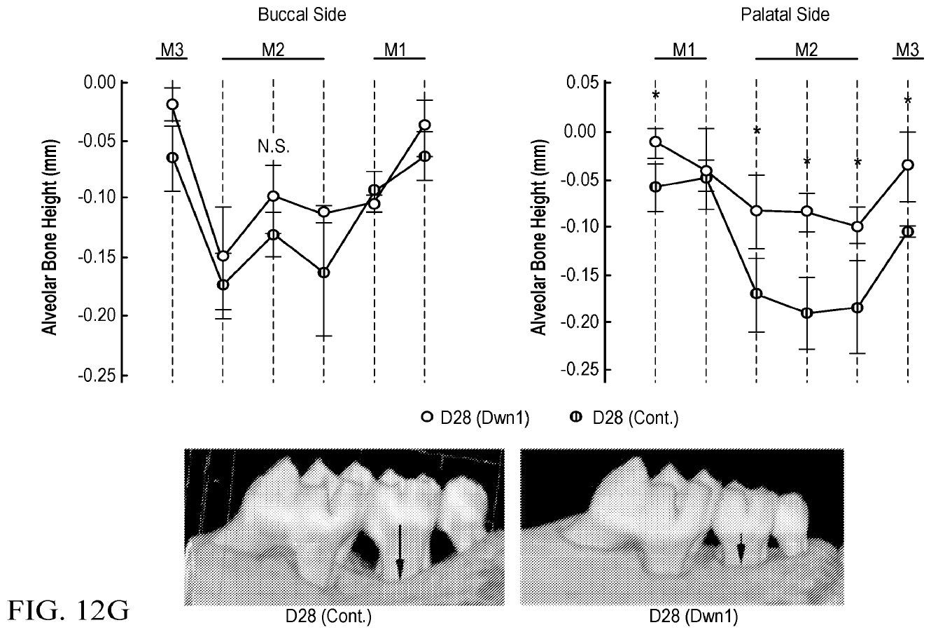

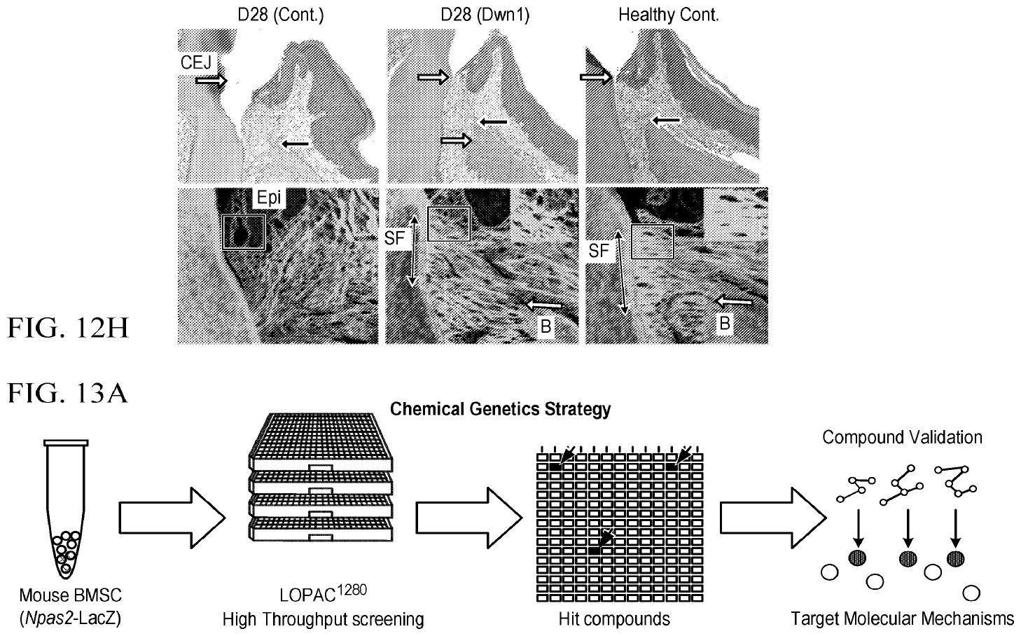

The selected highest hit compound Dwn1 (Reserpine) of Example 2 was applied to the well established ligature-induced periodontitis model with modification. After periodontitis was established at Day 14, the ligature was revoved from mouse molar mimicking non-invasive periodontits treatment: scaling and root planing (SRP) (FIGS. 12A-12D). Dwn1 was formulated in ABR LLC's propritery trans-epithelial deformable nano-scale vesicle (DNV) (57) and topically adminstered to the palatal gingival tissue once a week (FIG. 12E). The experimental group demonstrated the normalized gingival epithelium (FIGS. 12F, 12H). Alveolar bone regeneration was demonstrated by microCT at the Dwn1-treated plalatal side but not at the untreated buccal side (FIG. 12G). In the Dwn1 group, not only alveolar bone but also periodontal collagen with Sharpey's fibers appeared to be reconstructed (FIG. 12H). The in vivo regenerative efficacy of Dwn1 (Reserpine) was strongly indicated.

### Example 9

### Screening Npas2 Downregulating Compounds

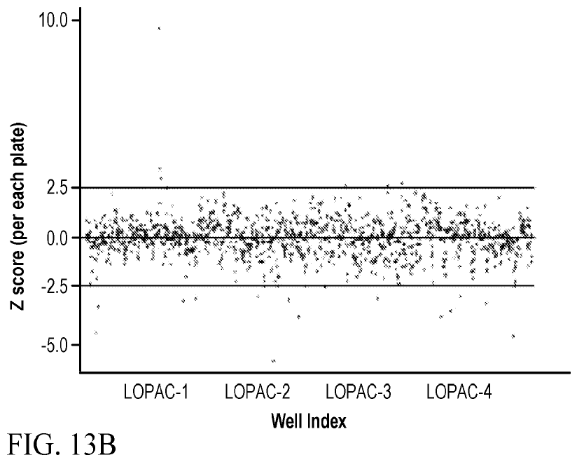

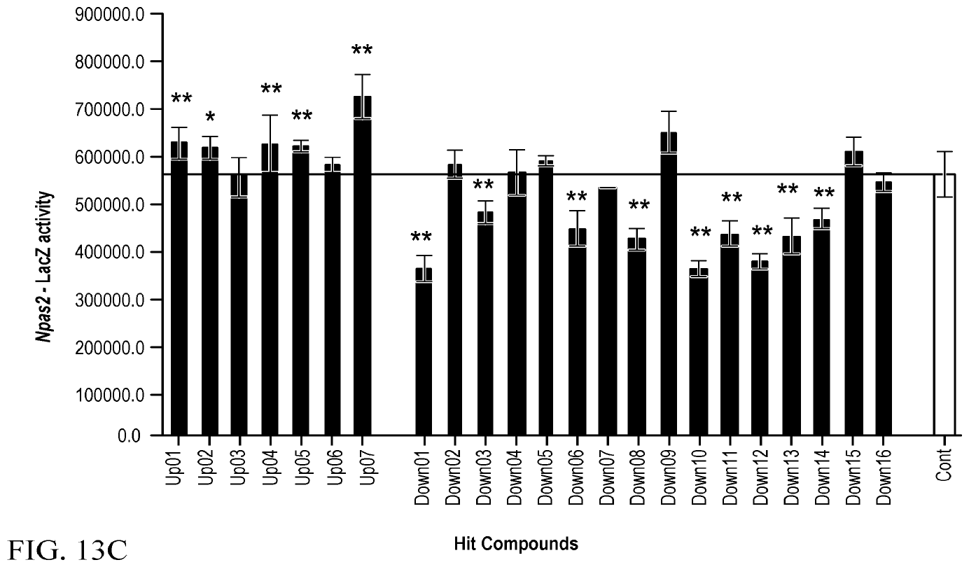

Femur BMSC derived from Npas2−/− mouse was previously characterized for the expression of LacZ, as described by Hassan, N., et al., (2017) Titanium biomaterials with complex surfaces induced aberrant peripheral circadian rhythms in bone marrow mesenchymal stromal cells. PLoS One 12, e0183359, which is incorporated by reference herein in its entirety, which was used for high throughput screening of LOPAC 1280 (FIG. 13A). The output data of screening analyzed for the Z score >2.5 or <−2.5 resulted in a total of 24 hits: 7 Npas2-upregulation and 16 Npas2-downregulation compounds (FIG. 13B). The validation study identified a total of 14 compounds (FIG. 13C), which were subjected to the chemical genetics analysis. Npas2 upregulating compounds were found to decrease intracellular cAMP or stimulate the alpha2 adrenergic receptor. By contrast, Npas2 down regulating compounds stimulate or accumulate cAMP, or induce cAMP response element binding (CREB) activation (Table 3).

TABLE 3
Npas2 Downregulating Compounds
Npas2 downregulation

| | | | | Description | |
|---|---|---|---|---|---|
| | | | | and Relevant | % |
| Compounds | | Class | Action | Functions | Activity * |
| Down01 | Brefeldin | Cyto- | Inhibitor | Fungal metabolite | −45.4 |
| | A | skeleton/ | | that disrupts | |
| | | ECM | | the structure and | |
| | | | | function of the golgi | |
| | | | | apparatus | |
| Down03 | Colchicine | Cyto- | Inhibitor | Prevents tubuline | −29.7 |
| | | skeleton/ | | polymerization | |
| | | ECM | | Potenciate PGE1 | |
| | | | | stimulation of cAMP | |
| | | | | formation | |
| Down06 | AC-93253 | Hormone | Agonist | Potent, cell | −25.1 |
| | iodide | | | permeable, subtype | |
| | | | | selective retionic | |
| | | | | acid receptor (RARa) | |
| | | | | agonist | |
| Down08 | Diphenyl- | Nitric | Inhibitor | Endothelial nitric | −18.6 |
| | eneiodon- | Oxide | | oxide synthase | |
| | ium | | | inhibitor | |
| | chloride | | | Inhibit cell Redox | |
| | | | | metabolism; | |
| | | | | Accumulate cAMP | |
| Down10 | Podophyllo | Cyto- | Inhibitor | Antineoplastic | −15.6 |
| | toxin | skeleton/ | | glucoside; inhibitor | |
| | | ECM | | of microtuble | |
| | | | | assembly Induce | |
| | | | | CREB activation | |
| Down11 | 5175348 | Cyto- | Inhibitor | Disrupts microtubles | −12.5 |
| | | skeleton/ | | by binding to beta- | |
| | | ECM | | tubulin | |
| Down12 | THAPSIG- | Intra- | Releaser | Potent, cell | −43.5 |
| | ARGIN | cellular | | permeable, IP3- | |
| | | Ca++ | | independent | |
| | | | | intracellular Ca++ | |
| | | | | releaser; Increase | |
| | | | | intracellular | |
| | | | | cAMP | |
| Down13 | PD-166285 | Kinase/ | Inhibitor | Broad spectrum | −35.5 |
| | hydrate | Phos- | | protein tyrosine | |
| | | phatase | | kinase inhibitor | |
| | | | | Inhibit Src and FGFR | |
| | | | | kinases | |
| Down14 | PD-173952 | Kinase | Inhibitor | Inhibit Src family | −33.0 |
| | | | | kinases | |

* Activity against negative controls in LOPAC screening

### Example 10

### Identifying Small Molecule Compounds Modulating Npas2 Expression by High Throughput Screening (HTS)

Culture medium will be dispensed into 384-well plates using the MultiDrop Combi system and compounds from the selected library will be applied using automated Biomek FX system Immortalized Npas2-LacZ MSC (1,500 cells per well) will be dispensed to each well and incubated at 37° C. for 36 hours (the peak expression time of Npas2). Then, MSC will be incubated with the LacZ detection agent (Beta-Glo, Promega, Madison, Wis.) and beta-galactosidase activity will be determined by luminometry. The luminometer data will be analyzed on CDD Vault algorithm (Collaborative Drug Discovery Vault, Burlingame, Calif.) for initial identification of suppressors as Z-score <−2.5. Primary MSC will then be incubated with initial hit compounds and stained with Calcein AM/Hoechst 33342 for a high throughput spinning disk confocal microscope (ImageExpress^{Confocal}, Molecular Devices, San Jose, Calif.). The viability will be determined by the number and size of cells. The hit compounds will be determined by Npas2 Z-score (<−2.5) and cell viability Z-score (>−2.5).

### Validation

The hit compounds will be dispensed on triplicated 384-well plates and primary MSC with Npas2-LacZ reporter gene will be added. After 36 hours of incubation, beta-galactosidase activity will be determined. The validated compounds must show statistically less Npas2-LacZ expression than untreated controls for p<0.01 by Student's t test.

### Titration for EC and IC

The validated hit compounds will be titrated from 100 μM to 0.2 nM in 20 wells of triplicated 384-well plates and primary Npas2-LacZ MSC (1,500 cells per well) will be dispensed. After 36 hours of incubation, the cell count and beta-galactosidase activity will be determined. The range of compound concentrations with significant Npas2-LacZ down-regulation and reduction of cell count will be the EC range and IC range, respectively. The final hit compounds with a Minimal Therapeutic Index of EC₅₀≥10×IC₅₀ will be identified.

### Sex as Biological Variable

No sex differences were observed in circadian clock behavior of isolated skeletal cells, as described by Okubo N, et al., PLoS One. 2013; 8(11):e78306, which is incorporated by reference herein in its entirety. It is proposed to use the single sex MSC for HTS (vertebrate animals)

### Anticipated Results

From approximately 40,000 compounds, it is anticipated that 40 (or 0.1%) hit compounds down-regulating Npas2 will be identified and approximately 100 compounds will be validated. Hit compounds will be further eliminated through the EC and IC index. It is anticipated that 20 candidates will be further analyzed.

### Potential Problems and Alternative Plans

For HTS, it is proposed to use immortalized MSC. The allelic genomic PCR will be performed after 5 passages to ensure the stability. Although unlikely, genetic shift or other mutations may occur. If needed, a new batch of primary MSC from Npas2-LacZ mice will be used.

### Example 11

### Establishing the Efficacy of Npas2 Suppressing Compounds for Accelerated Osteogenic Differentiation In Vitro

### ALP Expression Assay and In Vitro Mineralization Assay

Wild type male and female mouse MSC will be cultured with DMEM supplemented with 10% FBS and 1% antibiotics under 5% CO₂ at 37° C. After reaching semi-confluence, culture medium will be replaced with osteogenic medium (100 nM dexamethasone; 10 mM beta-glycerophosphate; 50 μM ascorbic acid) containing one of hit compounds (0.1, 1 and 10 μM in 0.1% DMSO). It must be noted that exposure to dexamethasone synchronizes MSC. At Day 7 of culture, the enzymatic ALP activity in the cell lysate will be measured using a commercially available kit (e.g. Abcam ab83369). The colorimetric data will be converted to ALP enzyme activity (n=3 per compound). Separately, at Day 21, in vitro mineralization will be assessed following standard protocol (i.e. ARed-Q, Sciencell Research Laboratories, Carlsbad, Calif. or QuantiChrome Calcium Assay Kit, BioAssay Systems, Heyward, Calif.). The average Alizarin Red S concentration or Ca++ concentration (n=3) will be obtained.

### Osteogenic Differentiation-Related Gene Expression

The “candidate” compounds will be characterized for time-course in vitro osteogenic differentiation. Wild type MSC exposed to the “candidate” compounds identified from the above experiments at the best concentration for the accelerated osteogenic differentiation will be used to isolate total RNA samples at culture period of 3, 7, 14 and 21 days. Real time PCR will be performed for Runx2, Osx, Ocn, Bmp2, Bmpr2, Col1a1, Opn, as well as Gapdh as control.

### Controls

The quantitative values of ALP, in vitro mineralization and osteogenic gene expression for MSC with 0.1% DMSO as untreated control will be obtained. For a positive control, it is proposed to supplement the osteogenic medium with BMP-2 (100 ng/ml).

### Data Analysis

The all assay data including untreated control and positive control will be ranked as 1 being the strongest. Then, compounds with the combined ranks that are above the rank of untreated MSC will be identified. From this list, the “candidate” compounds will be selected as the strongest 5 combined ranks. It is anticipated that the candidate compounds that may exhibit equivalent activities as the BMP-2-treated positive control.

### Sex as Biological Variable

Bone volume and structure are sexually dimorphic and male MSC formed more mineralized nodules than female MSC. The intrinsic molecular differences between male and female MSCs have been suggested. It is proposed to use male and female MSC (Vertebrate Animals)

### Anticipated Results

It is anticipated that the hit compounds will be evaluated successfully by (1) bone turnover marker, ALP expression and (2) in vitro mineralization for the demonstration of accelerated osteogenic differentiation. The ranking protocol should identify the candidate compounds. It is anticipated that most of compounds will generate statistically greater ALP and in vitro mineralization data than untreated control. The top-ranking compounds may achieve equivalent levels of BMP-2-derived osteogenic differentiation. Those compounds with the high ranks for osteogenic differentiation-related gene expression will be included. The “candidate” compounds should demonstrate the accelerated in vitro osteogenic differentiation through coordinated time-course expression of osteogenic genes. The top 10 “candidate” compounds will be selected for in vivo studies to demonstrate the efficacy of Npas2 suppressing compounds for alveolar bone regeneration in mouse ligature-induced periodontitis model.

### Potential Problems and Alternative Plans

Effective dose may vary. If EC ranges from identified small molecule compounds modulating Npas2 expression by HTS are outside of the proposed dose range of 0.1 to 10 μM, the compounds will be tested at a customized concentration range and if needed subjected to medicinal chemistry. It is noted that human and mouse MSC respond differently to BMP-2. A highly physiological osteogenic differentiation cocktail containing a physiological centration of BMP-2 previously was developed through feedback system control algorithm, as described by Honda Y, et al., Sci Rep. 2013; 3:3420, which is incorporated by reference herein in its entirety. It is proposed to use conventional BMP-2 (100 ng/ml) medium for positive control; however, if needed, alternative positive control cocktails may be used.

### Example 12

### Demonstrating the Efficacy of Npas2 Suppressing Compounds for Alveolar Bone Regeneration in Mouse Ligature-Induced Periodontitis Model

Minimally invasive surgical debridement with or without a recombinant growth factor has shown similar radiographic bone fill of small infra-bony pockets. An intrinsic environment was suggested to support alveolar bone regeneration. The postulated chronotherapy may not induce ectopic bone formation but support the host's healing environment. To test this hypothesis, the top 5 candidate compounds will be applied to the modified mouse ligature-induced periodontitis model and the efficacy of alveolar bone regeneration in vivo will be determined.

### DNV Formulation

Water-soluble or lipid-soluble small compounds are dissolved in either aqueous or lipid components. Using microfluidics, aqueous and lipid components will be mixed to generate DNV as described in the PCT/US2016062552, which is incorporated by reference herein in its entirety. DNV will be provided as freeze-dried powder.

### Mouse Periodontitis

Both male and female mice will be used. To induce periodontitis, a 5.0 silk suture will be placed around the maxillary second molar. After 14 days, the suture will be removed mimicking the non-surgical debridement treatment: Scaling and root planing (SRP). The candidate compound in DNV will be reconstituted in purified and sterilized water and topically applied to palatal gingiva (as shown in FIG. 12E).

### Data Analysis

At day 28, mouse maxillae will be harvested and subjected to microCT (n=20/group) and bone morphometric analysis with calcein injections (n=10/group) as well as decalcified histology (H&E and picrosirius red staining n=10/group). In addition, the serum level of bone turnover markers (Tracp5b; CTX; P1NP and ALP) will be determined.

### Sample Size

From the prior microCT data (FIG. 12G), a sample size of 20 is proposed in order to reach the power of 0.8.

### Sex as Biological Variable

Periodontitis has a documented sexual dichotomy with higher prevalence in men, potentially through sex difference in innate immunity among others. It is proposed to use male and female mice in the present Example to demonstrate the efficacy of Npas2 suppressing compounds for alveolar bone regeneration in mouse ligature-induced periodontitis model. (Vertebrate Animals).

### Anticipated Results

The primary outcome of alveolar bone regeneration will be assessed by microCT. The supporting evidences will be provided from histomorphometry, histology and serum markers. The exploratory outcome on the reconstruction of gingival/PDL connective tissue will be assessed by confocal laser scanning microscopy of picrosirius red stained histology. It is anticipated that the top 5 compounds will be identified for testing in Example “Phase II”.

### Potential Problems and Alternative Plans

It is noted that drug formulation of candidate compounds requires detailed optimization. Within the scope of Phase I (the herein proposed Examples), it is proposed to use DNV trans-oral epithelial drug formulation for all candidate compounds. The optimal drug formulation will be addressed in Example 13 (Phase II).

### Example 13

### Phase II

In this Example, it is planned to determine (1) the final drug formulation; (2) the efficacy in periodontitis of dogs; and (3) the safety of the small compound-based chronotherapy for periodontal and alveolar bone regeneration.

A large animal (dog) study is planned, as described by Kol A, et al., Companion animals Translational scientist's new best friends. Sci Transl Med. 2015; 7(308):308ps21 and Arzi B, et al., Craniomaxillofacial Disorders and Solutions in Humans and Animals J Dent Res. 2018; 97(4):364-70, each of which is incorporated by reference herein in its entirety.

### Example 14

### Role of Npas2 in Craniofacial Tissue Regeneration

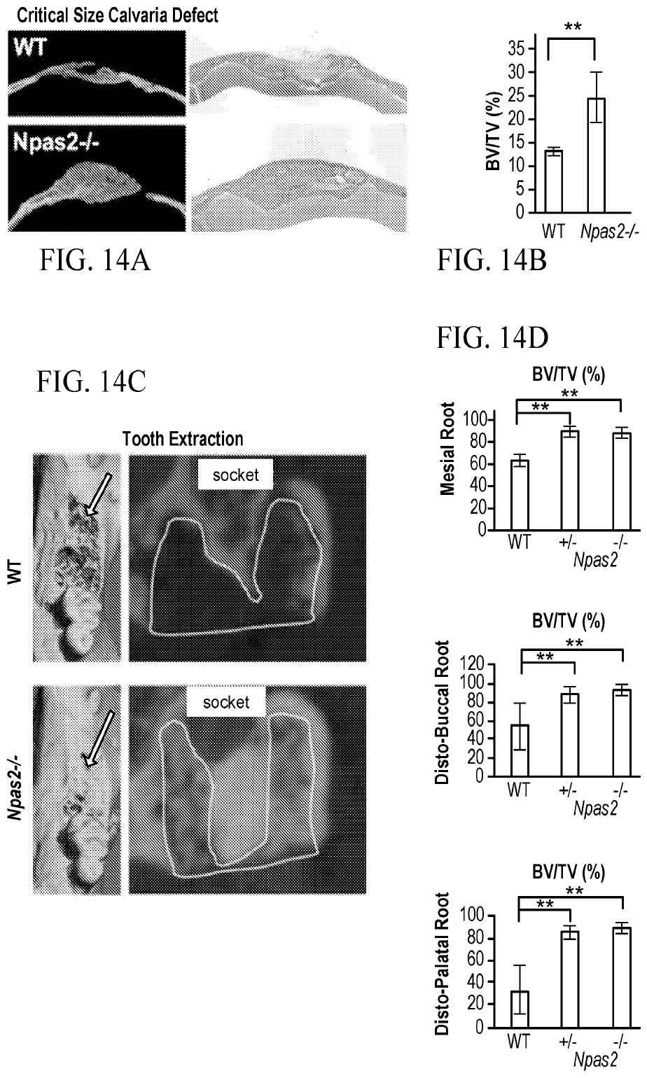

Extensive injury and chronic inflammation often result in wound repair with fibrosis, which prevents tissue regeneration. Thus, it was hypothesized that suppressing “wound repair gene Npas2” may lead to the wound healing toward tissue regeneration. The critical size defect created in the calvaria of Npas2 KO mice was healed with robust regeneration of bone and bone marrow tissues with the presence of low dose BMP-2 (325 ng following) (FIGS. 14A, 14B). Similar to the calvarial defect, bone formation in the extraction socket undergoes intramembranous ossification without cartilage precursor tissue; but post-tooth extraction bone regeneration is limited to the bottom half of the alveolar bone socket. Currently, the only other way to fill the upper half of the extraction socket is to use regenerative agents such as BMP-2. Npas2 KO mice uniquely demonstrated over 80%-100% of the tooth extraction socket filled with new bone (FIGS. 14C, 14D) without exogenously applied BMP or stem cells.

### Example 15

### Alveolar Bone Loss by Periodontitis was Regenerated in Npas2 KO Mice

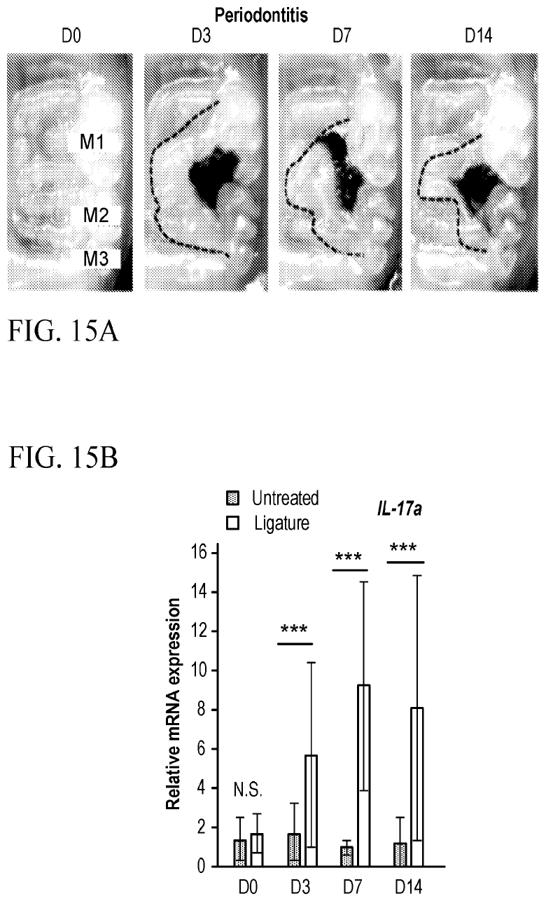

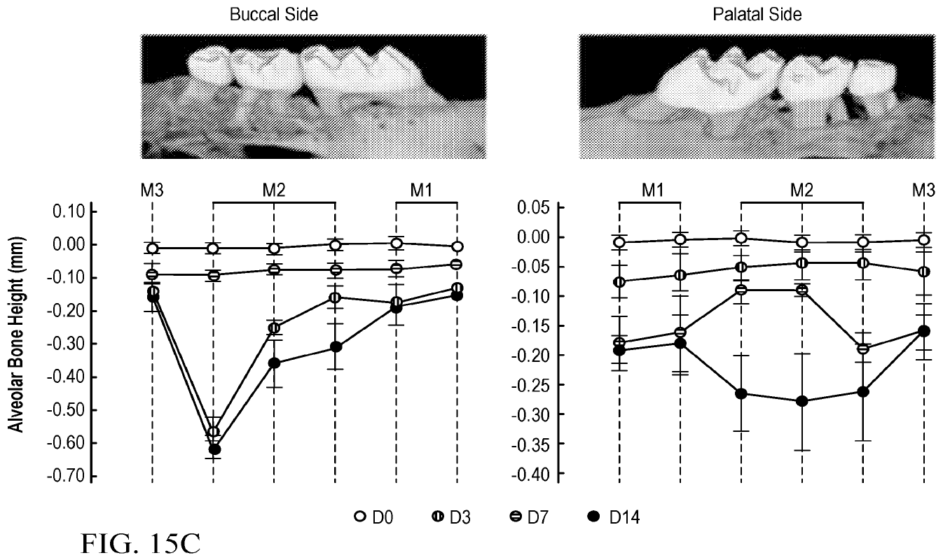

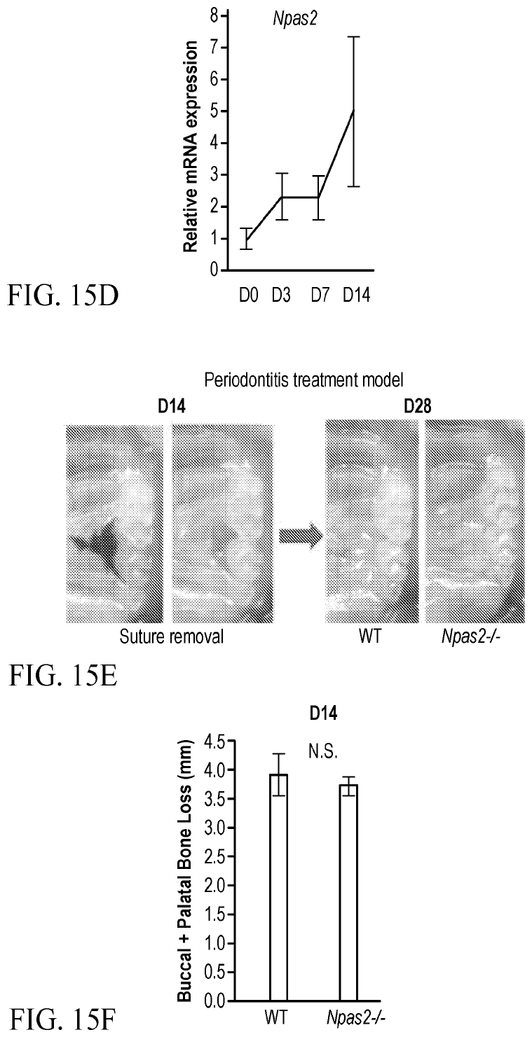

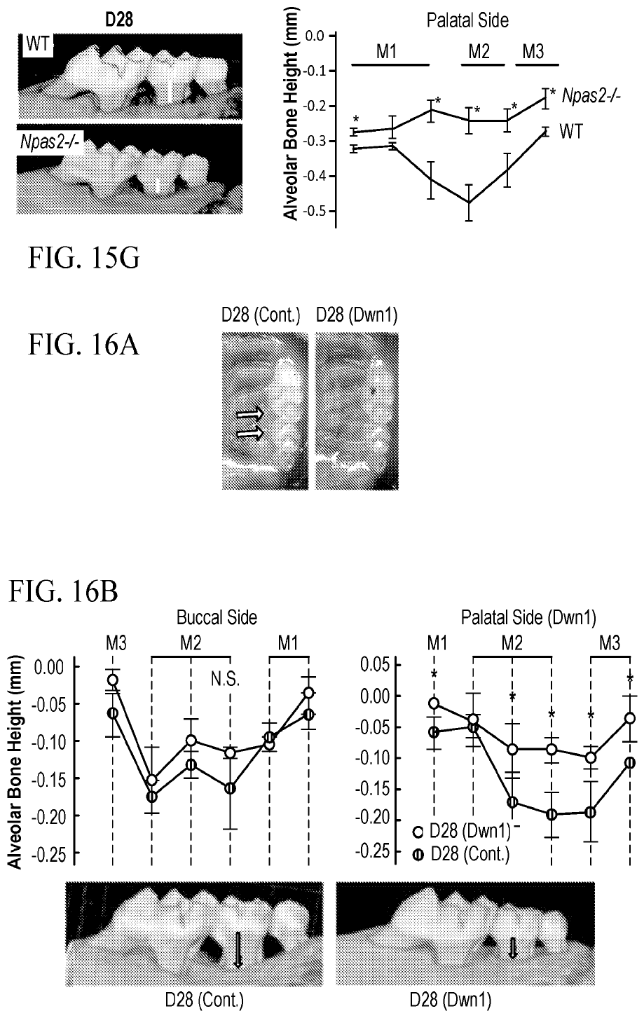

Periodontitis is a chronic inflammation induced by dysbiosis of oral microbial pathogens combined with discordant oral barrier immunity. A ligature placement around maxillary molar was shown to induce periodontitis in mice and this model had been used to characterize the oral microbial behaviors, gingival barrier immune reactions and aggressive alveolar bone resorption (FIGS. 15A-15C). In this mouse model, it was found that the Npas2 expression level in the affected gingiva tissue progressively increased (FIG. 15D). The current conventional treatment is to mechanically remove dental plaque and calculus from periodontal pocket by scaling and root plaining (SRP). SRP was mimicked by removing the suture at day 14 and monitor the healing for 2 weeks (D28). Gingival inflammation subsided (FIG. 15E) but the lost alveolar bone height (FIG. 15F) was not recovered in WT mice (FIG. 15G). When this periodontitis model was applied to Npas2 KO mice, alveolar bone loss at D14 was indistinguishable with WT mice (FIG. 2F). However, after mimicked “SRP” treatment, alveolar bone height significantly regained in Npas2 KO mice (FIG. 2G).

### Example 16

### Small Chemical Compound Suppressing Npas2 Regenerated Alveolar Bone in Mouse Periodontitis

UCLA Molecular Screening Shared Resources (MSSR) is a fee-for-service core facility to support high throughput screening (HTS) of drug-ready chemical compounds. MSSR is a unique facility for an academic institution, which stores over 200,000 compounds in various libraries and arrayed genome wide sets of CRISPR, cDNA, shRNA and siRNA (mssr.ucla.edu). The inventors performed multiple HTS using mouse MSC and fibroblasts carrying Npas2-LacZ reporter system. The two-fold objectives of HTS were to [1] elucidate the mechanism of Npas2 modulation through chemical genomics analysis; and [2] address if chemical compound(s) suppressing Npas2 may be capable of regenerating alveolar bone.

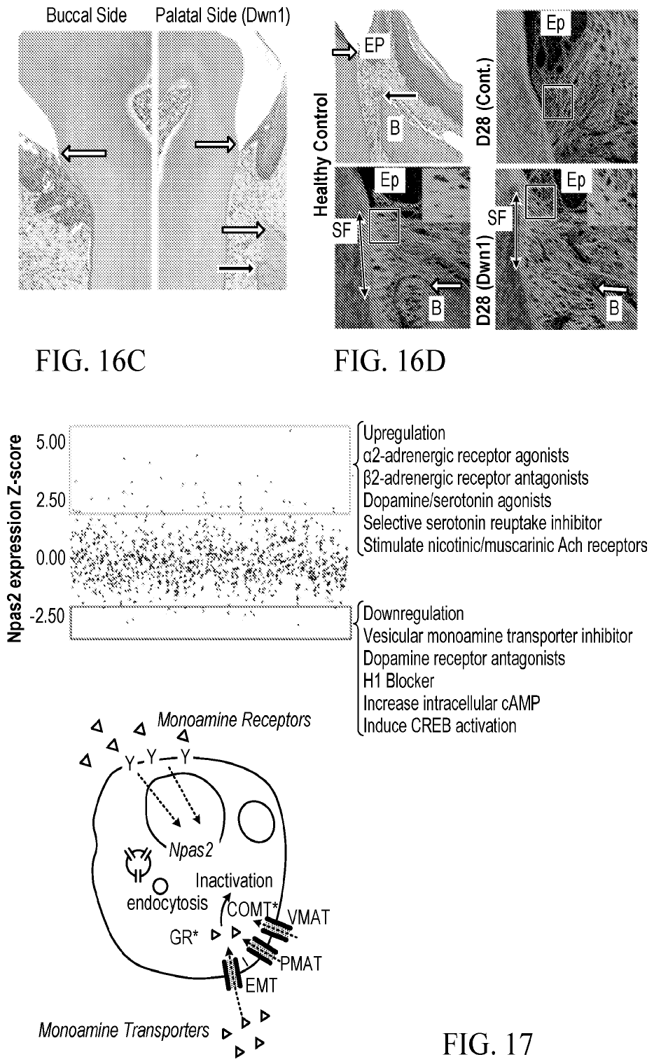

From FDA Approved Drug Library (1120 compounds) and LOPAC Library (1280 compounds), a list of chemical compounds that modulated Npas2-LacZ expression was identified. For the latter objective, one compound that down-regulated Npas2-LacZ expression (Dwn1) was selected and applied to the mouse periodontitis/treatment model (FIGS. 15E-15G). Dwn1 was formulated for topical application to palatal gingiva. After 14 days of periodontitis induction, the ligature was removed and Dwn1 was topically applied to the palatal gingiva once a week (FIG. 16A). At D28, the significant alveolar bone regeneration was observed only at the palatal side, where Dwn1 was applied (FIGS. 16B, 16C). There were additional unexpected observations that gingival and periodontal ligament collagen fibers were reorganized with potential regeneration of Sharpey's fibers (FIG. 16D). These results provided the basis for pursuing clinical and translational development of viable options for oral regenerative chronotherapy.

### Example 17

### Chronobiological Hypothesis from HTS-Chemical Genomics

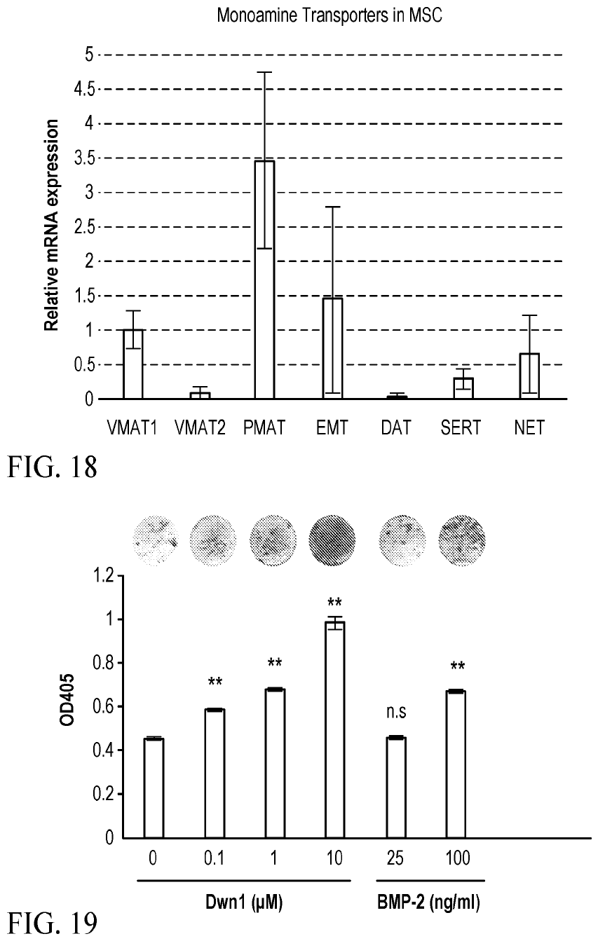

The chemical genomics analysis of the HTS data identified a cluster of drugs targeting the monoamine-related transporters and receptors (FIG. 17). Monoamines (i.e., noradrenaline, serotonin, dopamine, histamine) have been extensively characterized as neurotransmitters and a large number of drugs have been developed targeting this axis. It must be noted that their receptors have been found in non-neuronal cells such as MSC and monoamine-induced signal transduction was postulated to regulate the osteoblast growth and differentiation. In addition, monoamine transporters were also reported in non-neuronal cells and all monoamine transporters were found expressed by MSC (FIG. 18).

In fact, Dwn1 was inhibitor of one of the pan-monoamine transporters, which downregulated Npas2 and exhibited tissue regenerative activity (FIGS. 16A-16D), suggesting that the regulatory axis of monoamine receptors and transporters may modulate the Npas2 expression leading to the induction of regenerative capacity. Harnessing the HTS/chemical genomics data, the chronobiology of tissue regeneration will be elucidated in a study focusing on the monoamine-pathway axis and downstream Npas2 expression. This study should establish the mechanism of action (MOA) of the proposed chronotherapy for periodontal tissue regeneration.

### Example 18

### “Chronotherapy” for Periodontal Tissue Regeneration

Circadian synchronization affects numerous molecular, physiological and biological processes. Dysregulation of circadian rhythm was reported in neuropsychiatric diseases as well as in metabolic diseases and cancer. There are increasing reports suggesting that circadian clock molecules can be a therapeutic target; e.g., Bmal1 for malignant pleural mesothelioma and Alzheimer's disease. Therapeutic potential of small molecules modulating circadian systems has been proposed as a novel approach of “chronotherapy”. This project proposes to develop innovative small chemical compound-based chronotherapy for dental tissue regeneration. To this end, the chemical space of monoamine-pathway axis will be fully explored for modulating the Npas2 expression. The following approach will be used:

### Study 1. Construct Chemical Space Specific HTS Compound Library and Identify Chronotherapeutic Compounds for Modulating Npas2 (Performed by ABR LLC)

Rationale and Objectives. The preliminary HTS identified a cluster of drugs targeting the monoamine-related transporters and receptors, which affected the Npas2 expression in MSC. The objective of this study is to carry out focused HTS using a customized compound library that is composed of selected small molecule compounds in the chemical space of monoamine transporters and monoamine receptors. This study proposes to [1] construct a focused chemical compound library customized for the chemical space of monoamine-pathways; [2] complete HTS; [3] validate Npas2 expression and MSC osteogenic differentiation; [4] determine the effective concentration (EC) and inhibitory concentration (IC).

ABR LLC has an incubator space in the UCLA California NanoSystems Institute (CNSI), which also houses the UCLA drug screening core facility, MSSR. ABR LLC has full access to MSSR, where the chemical space specific library will be customized and HTS will be performed. ABR LLC will complete the objective of this project.

Exp 1 (Chemical Space specific library). There are 7 monoamine transporters (FIG. 18). MSSR has a total of 51 chemical compounds targeting the monoamine transporters, which work as inhibitors. Separately, adrenergic receptors, dopamine receptors, serotonin receptors, muscarinic/nicotinic receptors and H1 receptors are targeted by a total of 283 compounds. These compounds are either agonists or antagonists. As a result, a total of 334 compounds will be included in the monoamine chemical space specific compound library. The compound library will be constructed in 384-well plates including vehicle only control wells for HTS.

Exp 2 (HTS). Culture medium will be dispensed into 384-well plates using the MultiDrop Combi system and compounds from the chemical space specific library will be applied using automated Biomek FX system Immortalized Npas2-LacZ MSC (1,500 cells per well) will be dispensed to each well and incubated at 37° C. for 36 hours (the peak expression time of Npas2). MSC will be stained with Calcein AM/Hoechst 33342 for a high throughput spinning disk confocal microscope (ImageExpress^{Confocal}, Molecular Devices, San Jose, Calif.) for the cell viability measurement, and then, incubated with the LacZ detection agent (Beta-Glo, Promega, Madison, Wis.). Beta-galactosidase activity will be determined by luminometry. The data will be analyzed on CDD Vault algorithm (Collaborative Drug Discovery Vault, Burlingame, Calif.) for Npas2 expression and cell viability to determine Z-score for each measurement. Npas2 Z-score threshold will be >2.5 and <−2.5 and cell viability Z-score threshold will be between −1.0 and +1.0 based on the preliminary data hereinabove.

Exp 3 (Validation). The compounds effective in modulating Npas2 will be dispensed on triplicated 384-well plates and MSC with Npas2-LacZ reporter gene will be added. After 36 hours of incubation, beta-galactosidase activity will be determined. The validated compounds must show statistically modulated Npas2-LacZ expression than untreated controls for p<0.01 by Student's t test.

FIG. 10 shows titration assays. Dwn1 was serially diluted from 100 μM to 0.2 nM and applied to MSC Npas2-LacZ. EC was determined by LacZ expression and IC was determined by cell viability using Calcein AM/Hoechst 33342 staining.

Exp. 4 (Titration for EC and IC). The validated hit compounds for suppressing Npas2 will be titrated from 100 μM to 0.2 nM in 20 wells of triplicated 384-well plates and Npas2-LacZ MSC (1,500 cells per well) will be dispensed. After 36 hours of incubation, the cell count and beta-galactosidase activity will be determined (FIG. 10). The range of compound concentrations with significant Npas2-LacZ down-regulation and reduction of cell count will be the EC range and IC range, respectively. The final hit compounds will be identified with a Minimal Therapeutic Index of EC₅₀≥10×IC₅₀. This study will also determine the optimal concentration of each hit compound.

Exp. 5 (Osteogenic differentiation). Npas2-suppressing hit compounds in 0.1% DMSO at the optimal concentration will be added to the conventional osteogenic culture for WT male and female mouse MSC. The degree of osteogenic differentiation will be determined by the ALP activity (e.g. Abcam ab83369) and in vitro mineralization (i.e. ARed-Q, Sciencell Research Laboratories, Carlsbad, Calif.) (FIG. 19). Furthermore, total RNA samples will be prepared at culture period of 3, 7, 14 and 21 days. Real time PCR will be performed for Runx2, Osx, Ocn, Bmp2, Bmpr2, Col1a1, Opn, as well as Gapdh as control. Controls: The quantitative values of ALP, in vitro mineralization and osteogenic gene expression of MSC will be obtained with 0.1% DMSO as untreated control. For a positive control, the osteogenic medium with BMP2 (100 ng/ml)_will be used.

Sex as biological variable. No sex differences were observed in circadian clock behavior of isolated skeletal cells. The use of the single sex MSC is proposed for Exp 1-4. Bone volume and structure are sexually dimorphic and male MSC formed more mineralized nodules than female MSC. The intrinsic molecular differences between male and female MSCs have been suggested. Male and female MSC will be used for Exp 5.

Anticipated Results. Chemical Genomics analysis based on the new chemical space specific HTS in this study should further determine the role of monoamine-related pathways in modulating Npas2 expression of MSC at high-resolution, which should lead to the mechanistic understanding of chronotherapy. The function of monoamine transporters in non-neuronal cells was recently investigated. The metabolism of norepinephrine in perivascular adipose tissue (PVAT) was investigated. PVAT adipocytes were found to express monoamine transporters: vesicular monoamine transporter (VMAT1, VMAT2), plasma membrane monoamine transporter (PMAT) and norepinephrine transporter (NET). Cellar accumulation of fluorescent norepinephrine analogue signal was markedly reduced by chemical inhibitors of VMAT, PMAT and NET in vitro. Therefore, it is anticipated that chemical compounds targeting monoamine transporters may modulate the monoamine concentrations in the culture medium affecting the function of monoamine receptors of MSC.

Separately, antagonists and agonists of monoamine receptors may directly modulate their function and influence the downstream signal transduction pathways. Serotonin antagonists have been associated with reduced hepatocellular regeneration, and serotonin secreted by platelets and inflammatory cells plays an important role in cutaneous wound healing. Recently, serotonin receptor agonist was shown to accelerate skin wound healing. The present study will determine the effect of agonists and antagonists on the Npas2 expression.

Chemical genomics analysis should determine the specific monoamine pathway to modulate the Npas2 expression. In particular, it is anticipated that a list of compounds that decrease the Npas2 expression will be obtained. These compounds are expected to increase osteogenic differentiation assessed by statistically greater ALP and in vitro mineralization as well as coordinated time-course expression of osteogenic genes. The top-ranking compounds may achieve equivalent levels of BMP-2-derived osteogenic differentiation. These hit compounds with minimal therapeutic EC/IC Index will be identified for chronotherapy development in the Phase II project.

Potential Problems and Alternative Plans. For HTS, it is proposed to use immortalized MSC. The allelic genomic PCR will be performed after 5 passages to ensure stability. Although unlikely, genetic shift or other mutations may occur. If needed, a new batch of primary MSC from Npas2-LacZ mice will be used.

### Study 2. Determine the Regulatory Role of Monoamine Receptors and Transporters on the Npas2 Expression and Osteogenic Differentiation in Human MSC (Performed by UCLA Laboratory)

Rationale and Objectives. The gingival tissue affected by the ligature-induced periodontitis revealed the progressive increase of Npas2 expression (FIG. 15D). Separately, it was found that MSC expressed monoamine transporters that were previously thought to be specific to neuronal cells (FIG. 18). The expression profile of adrenergic receptors of human MSC has been shown sensitively modulated by the culture environment. Therefore, it is further hypothesized that the environmental modulation of the expression of monoamine receptors and/or transporters affects the differentiation capacity of MSC through Npas2 expression. The objective of this study is to characterize the behavior of human MSC with the over-expression of monoamine-related molecules. [1] The open reading frame (ORF) expression plasmids of human monoamine receptors and transporters will be prepared in lentivirus vector. [2] Human MSC will be transduced to over-express each of monoamine receptors and transporters. [3] The Npas2 expression and osteogenic differentiation will be determined.

MPI's UCLA laboratory will carry out all proposed projects of Study 2. It must be noted that Study 1 and Study 2 projects complementally address the overarching goal.

Exp 1 (ORF expression plasmids). From the chemical genomics analysis and literature reviews, 7 monoamine transporters and 17 monoamine receptors were selected for this project (Table 4).

TABLE 4
Monoamine-axis molecules
Table. Monoamine-axis molecules

| Monoamine transporters | Human Gene |
|---|---|
| VMAT1 | SLC18A1 |
| VMAT2 | SLC18A2 |
| EMT | SLC22A3 |
| PMAT | SLC29A4 |
| NET | SLC6A2 |
| DAT | SLC6A3 |
| SERT | SLC6A4 |
| Monoamine receptors | Human Gene |
| Dopamine receptor D1 | DRD1 |
| Dopamine receptor D2 | DRD2 |
| Dopamine receptor D3 | DRD3 |
| Dopamine receptor D4 | DRD4 |
| 5-HT1A receptor | HTR1A |
| 5-HT1B receptor | HTR1B |
| 5-HT2A receptor | HTR2A |
| 5-HT2B receptor | HTR2B |
| 5-HT2C receptor | HTR2C |
| 5-HT3A receptor | HTR3A |
| 5-HT7 receptor | HTR7 |
| Histamine H1 receptor | HRH1 |
| Alpha2A adrenergic receptor | ADRA2A |
| Alpha2B adrenergic receptor | ADRA2B |
| Alpha2C adrenergic receptor | ADRA2C |
| Beta 1 adrenergic receptor | ADRB1 |
| Beta 2 adrenergic receptor | ADRB2 |

From the public genome-scale lentiviral expression library of human ORFs (50), the list of ORF expression vectors have been obtained. Using conventional 3^{rd} generation lentiviral vector production protocol (51, 52), the expression vectors of human monoamine related molecules will be constructed.

Exp 2 (Human MSC over-expressing monoamine-related molecules). Human MSC (iMSC3, Applied Biological Materials) will be transduced by the lentiviral vectors, which carry antibiotics (Blasticidin) resistant gene. The transduced cells will be selected by Blasticidin (5 μg/μ1: from the kill curve). The surviving MSC will be expanded in growth medium and confirmed the over-expression of transduced gene. The following experiments will be performed with the transduced MSC.

Exp 3 (Npas2 expression). MSC transduced with each of monoamine-related molecules will be examined for the expression of Npas2 using RT-PCR (53). Because Npas2 is a core circadian gene, MSC will be synchronized by forskolin (10 μM) for 2 hr. The RNA samples will be prepared every 4 hr from 24 hr to 72 hr after the synchronization.

Exp 3 (Osteogenic differentiation). The monoamine-related molecule transduced MSC will be subjected to osteogenic differentiation. In vitro osteogenic differentiation will be monitored by ALP enzyme activity and Alizarin Red staining for in vitro mineralization. The time course expression of osteogenic differentiation-related genes will be monitored by qPCR.

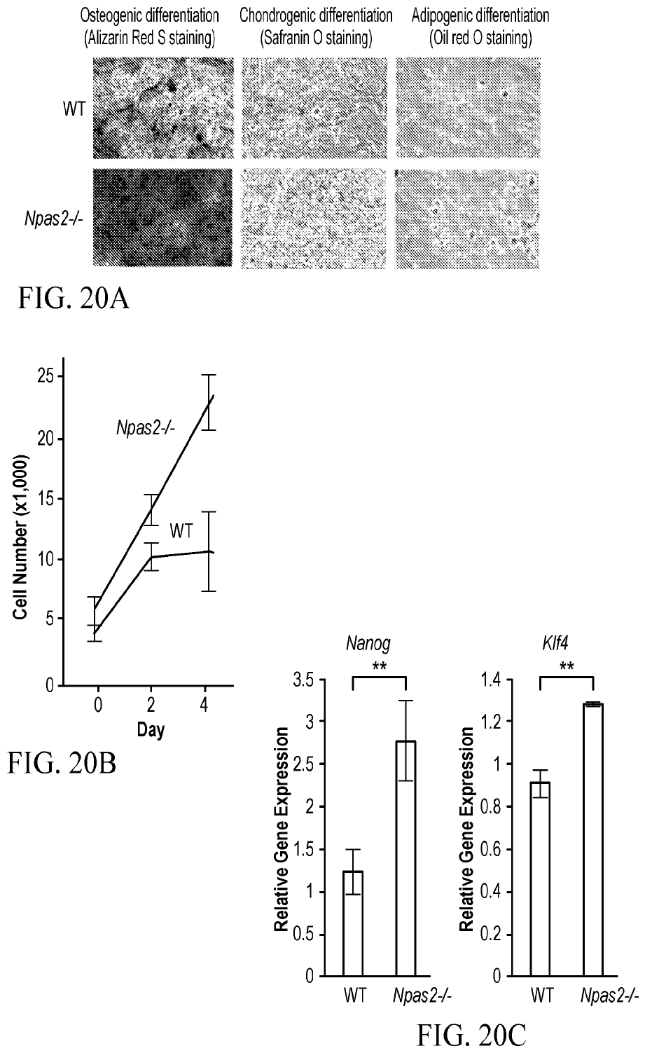

Exp 4 (Stem cell marker expression). MSC with Npas2 KO mutation exhibited increased multipotent differentiation, while maintaining the high level of stem cell markers: Nanog and Klf4 in the undifferentiated stage (FIGS. 20A-20C). MSC of dental origins have been implicated to play an important role in periodontal tissue regeneration. Thus, the effect of monoamine-related molecule over-expression on stem cell characteristics will be determined. The time course proliferation and the expression of mesenchymal stem cell markers, Nanog and Klf4 will be determined.

Sex as biological variable. The intrinsic molecular differences between male and female MSCs have been suggested. However, it is proposed to use the commercially available clonal MSC, which will be provided without the source information.

Anticipated Results. The monoamine-related increase in Npas2 expression is expected to decrease MSC's osteogenic differentiation. By contrast, MSC with decreased Npas2 expression is expected to behave similar to Npas2 KO MSC (FIGS. 20A-20C). The major goal of Study 2 is to determine which monoamine-related molecules effectively modulate (increase or decrease) the Npas2 expression. Interactions between serotonin and circadian system have been reported in SCN and in mood disorders. Dopamine has been shown to influence circadian expression of Npas2 in retina. The upregulation of alpha-adrenergic receptors has been suggested to increase Npas2 expression in MSC. The preliminary data herein suggested the involvement of pan-monoamine transporter in regulating the Npas2 expression (FIG. 10). Therefore, it is highly likely to identify the specific monoamine-related molecule(s) that regulate Npas2 expression.

The new identification of monoamine-related molecules in this project should immediately suggest a novel molecule mechanism of decreased regenerative capacity in the wound and chronic inflammation, where environmental modulation of monoamine-related molecules may play a pathological role, potentially through the downstream upregulation of Npas2. As such the MOA of the proposed chronotherapy will be established.

Potential Problems and Alternative Plans. Npas2 is one of the core clock genes. It is possible that other clock genes may be modulated by the monoamine-related molecules, which should be characterized.

The serum component in the cell culture contains the physiological concentration of monoamines. However, it is indicated, monoamines will be supplemented in the culture medium.

Phase I outcomes will efficiently identify monoamine chemical space specific compounds to suppress Npas2 with full characterization in vitro and will elucidate Npas2 chronobiology for stem cell differentiation. Study 1 (ABR) and Study 2 (UCLA) projects complementally address the overarching goal. MPIs will regularly communicate their progress and outcome to efficiently manage the proposed projects.

Phase II and commercialization plan may include: [1] optimal drug formulation of the candidate small molecules suitable for clinical application; [2] efficacy assessment toward the alveolar bone regeneration in the mouse periodontitis model; [3] pre-clinical efficacy and safety study using canine periodontitis; and [4] biocompatibility/safety assessment following ISO 10993-1 for FDA application.

### Example 19

### Small Molecule Inhibitor of Npas2 for Surgical Scar Prevention

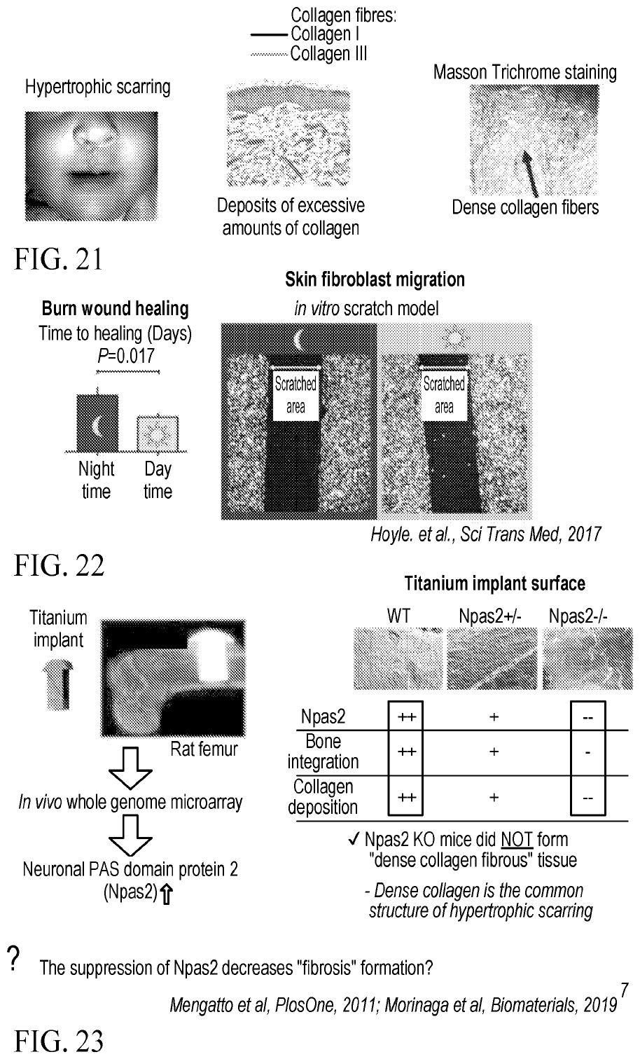

Despite much effort, effective therapeutics to minimize scarring have not been successfully developed. Worldwide, 100 million patients develop scars from elective and trauma operations alone each year. In 40-70% of surgical cases, patients experience hypertrophic scarring and a raised scar(s), caused by deposits of excessive amounts of collagen, wherein the collagen comprises dense collagen fibrous tissue (“fibrosis”). (FIG. 21). Current standard treatments for scar prevention are continuous corticosteroid injections, laser therapy, surgical excision, and/or radiation.

Circadian rhythms influence wound healing, as shown in FIG. 22 for burn wound healing and in an in vitro scratch model of skin fibroblast migration (as described by Hoyle et al., Sci. Trans Med 2017, which is incorporated herein by reference in its entirety).

In vivo microarray analysis of whole genome found NPAS2 was upregulated in in a rat femur into which a T-shaped titanium rod was implanted (FIG. 23 left) and Npas2 knockout (KO) (Npas2−/−) mice did not form dense collagen fibrous tissue on a titanium implant surface compared to wild type (WT) and Npas2+/−. (FIG. 23 right), as described by Mengatto et al, PlosOne, 2011; Morinaga et al, Biomaterials, 2019, each of which is incorporated herein by reference in its entirety. Npas2 KO improves wound healing in mouse model as described by Sasaki, et al. Anatomical Record, 2019, which is incorporated by reference herein in its entirety.

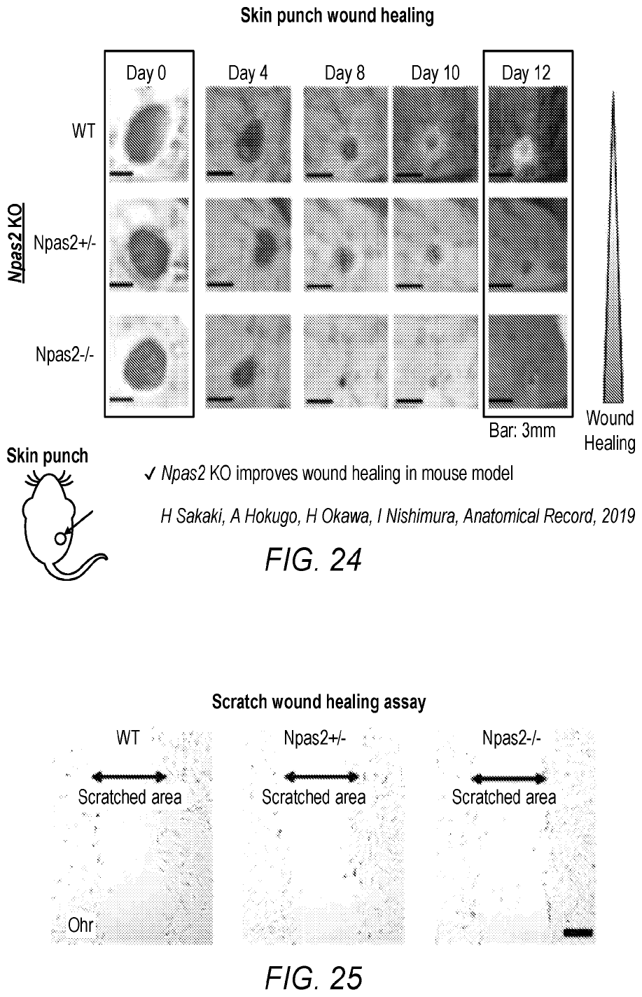

In vitro wound healing assays showed that Npas2 KO skin fibroblasts improves wound healing in vitro model (FIGS. 24-25 and FIG. 3C).

Example 2 above describes Npas2 suppressor high throughput screening, which identified ten compounds that downregulate Npas2 (shown in FIGS. 13A and 26).

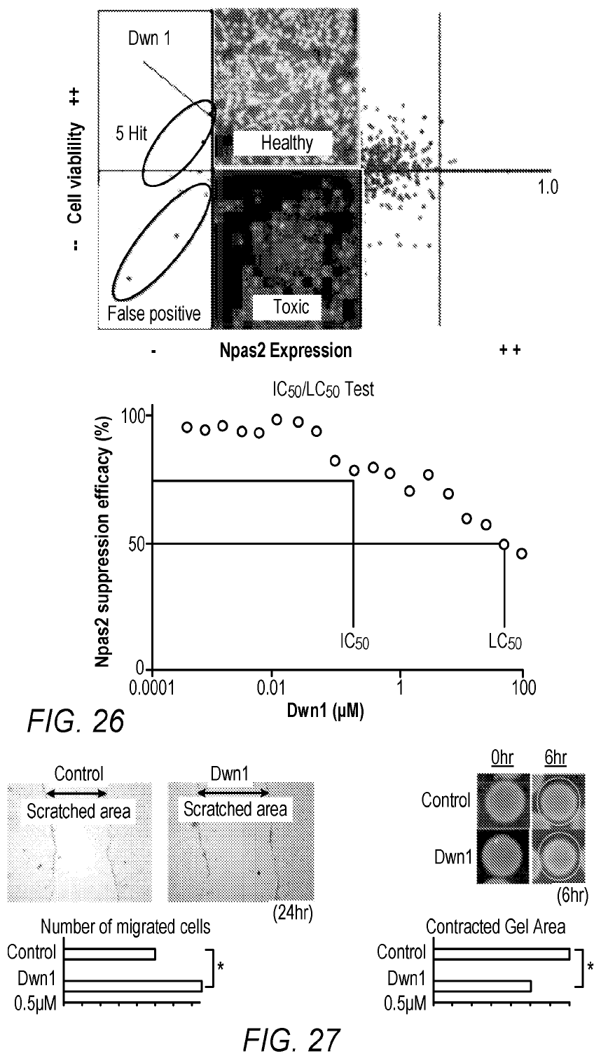

As described herein, a small molecule inhibitor of Neuronal PAS domain 2 (Npas2), Dwn1, improves wound healing and minimizes scarring. As shown in FIG. 27, Dwn1 accelerated (1) fibroblast migration m a scratch wound healing assay and (2) collagen gel contraction in vitro.

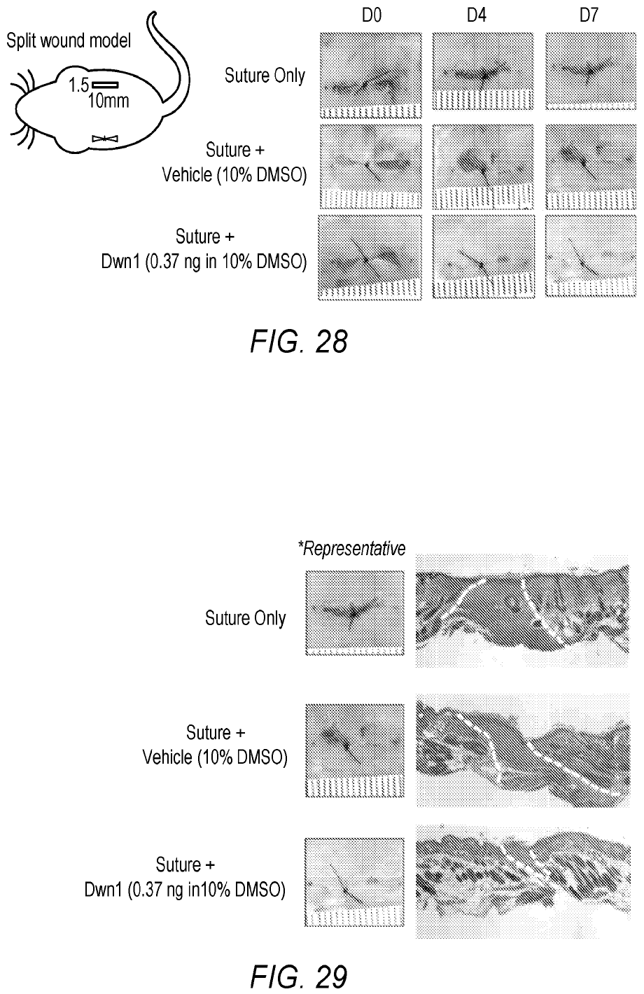

Dwn1 improved split wound healing with minimal scarring when administered to a wound for which a suture was used compared to both suture wound closure only and suture plus administration of vehicle (10% DMSO), as shown in FIG. 28. In addition, administration of Dwn1 to a wound closed with a suture decreased excessive collage deposition on or around the wound compared to using a suture only or suture and administration of vehicle (10% DMSO), as shown FIG. 29.

The small molecule inhibitor of Npas2 is expected to accelerate wound close and reduce scarring. Npas2 KO did not result in embryonic and developmental pathology. As such Npas chronotherapy, i.e., administration of an Npas2 inhibitor to suppress Npas2 and modulate circadian systems is proposed as a therapeutic agent to decrease collagen “fibrosis” formation and reduce scarring of wounds.

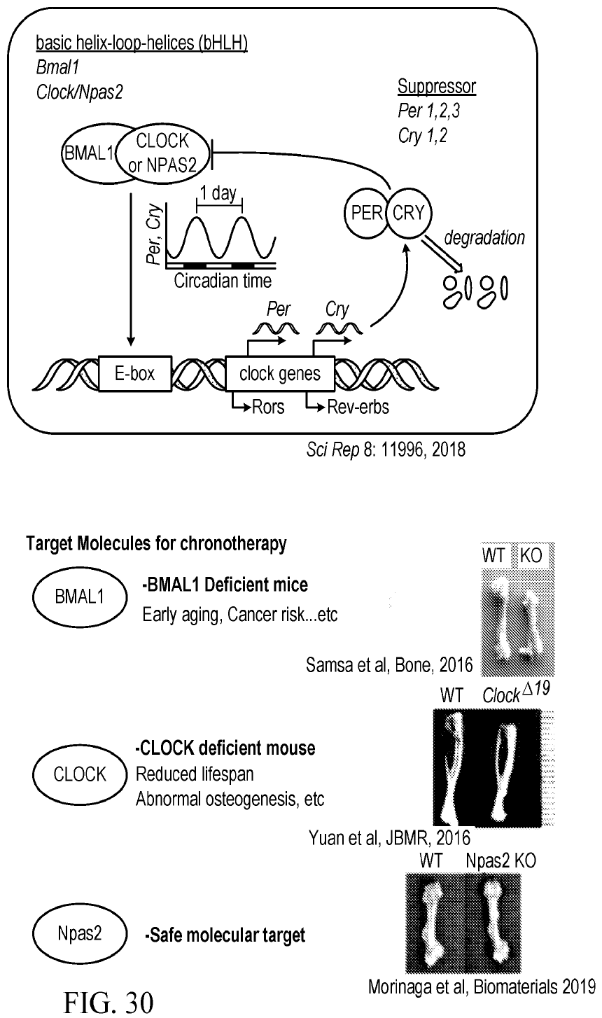

While there are target clock molecules for chronotherapy besides Npas2, such as Bmal1 or Clock, (FIG. 30), Npas2 has bene shown to be a safe molecular target: KO mutations of Bmal1 or Clock generated various pathological phenotypes in peripheral bone tissues and premature aging symptoms (sarcopenia, cataracts, organ shrinkage). Npas2 KO mutation, however, did not result in embryonic and developmental pathology of jawbone, vertebral and appendicular bones. The level of Npas2 expression in SCN is low and has little contribution to the central circadian rhythm.

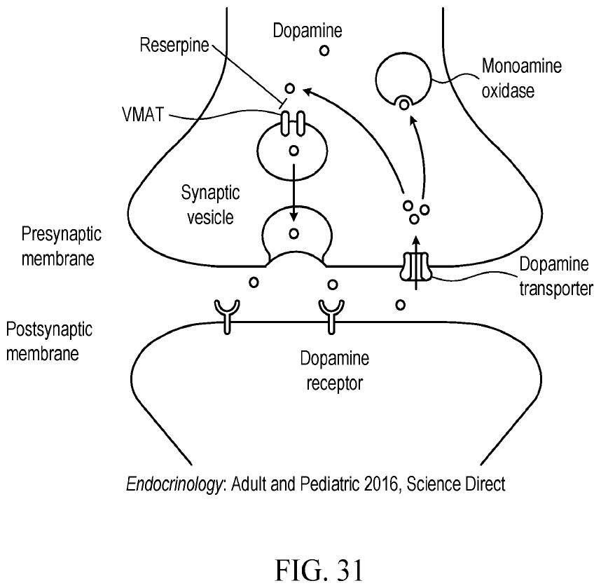

Reserpine blocks the vesicular monoamine transporters (VMAT) which is mostly expressed in neuroendocrine cells and neurons. (FIG. 31) Blockade of neuronal VMAT by Reserpine inhibits uptake and reduces stores of the monoamine neurotransmitters; norepinephrine, dopamine, serotonin and histamine in the synaptic vesicles of neurons. Transcription of the monoamine oxidase A (MAOA) promoter is regulated by the clock components BMAL1, NPAS2, and PER2. A mutation in Per2 in mice leads to reduced activity of MAOA.

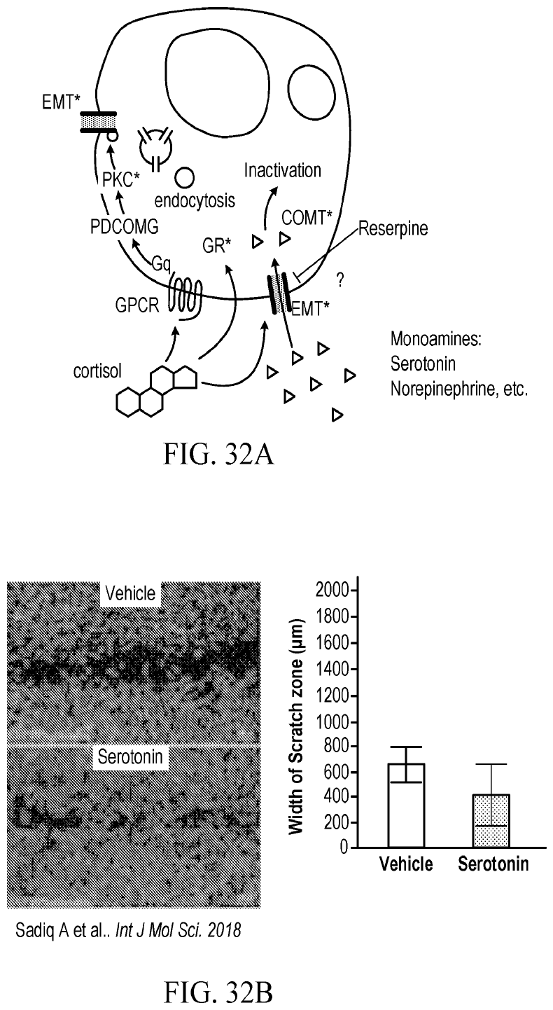

There are extraneuronal monoamine transporters (EMT) which are similar to VMAT and are expressed in various cells such as chondrocyte, and smooth muscle cells (FIGS. 30 and 32A). The width of a scratch zone was reduced when serotonin was administered in vitro compared to vehicle (FIG. 32B), as shown by Sadiq et al., Int J. Mol Sci 2028, which is incorporated herein by reference in its entirety. It is hypothesized that Reserpine blocks EMT in fibroblasts, leading to extracellular serotonin accumulation which accelerates fibroblast migration. (FIG. 32A).

### Example 20

### Murine Model of Surgical Scar Healing/Prevention

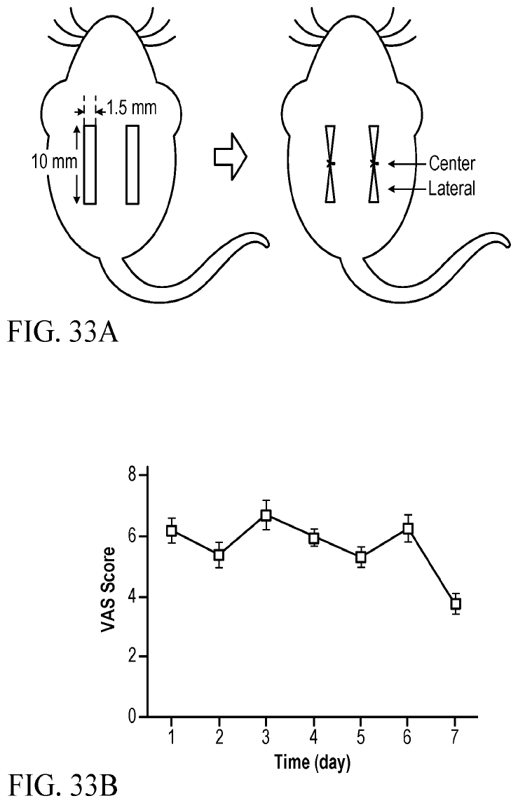

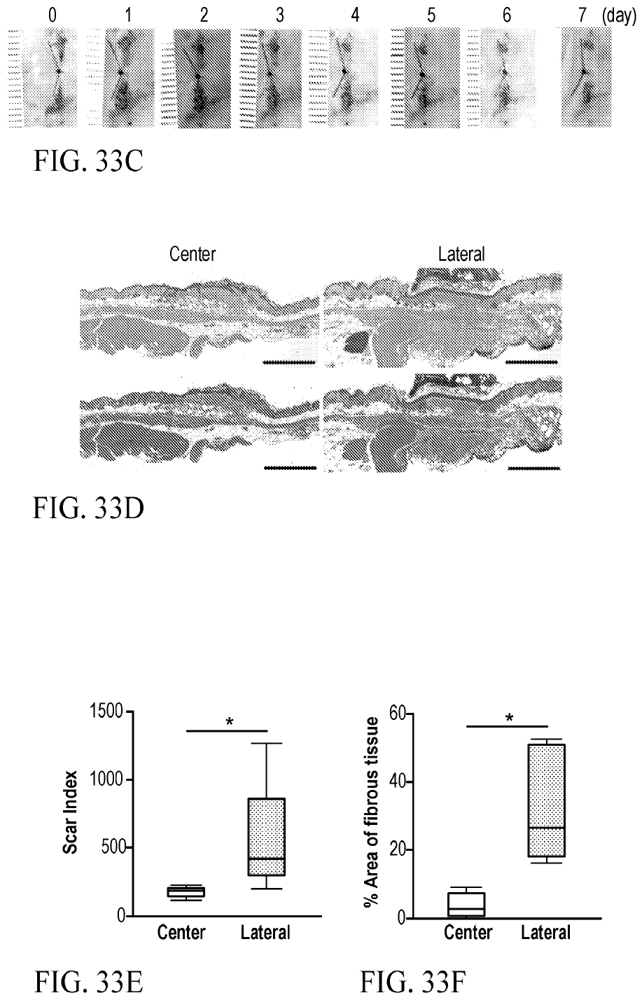

Vertical wounds (10×1.5 mm) were made on both left and right sides of the back of a mouse with a double-bladed scalpel. One ligation was performed at the center of the wound with 5-0 nylon suture. (FIG. 33A) Visual Analogue Scale (VAS) was scored everyday postoperatively until postoperative day 7 using gross images of the wounds/scars (FIG. 33A), and postoperative gross images of the wounds/scars were measured with a ruler. (FIG. 33C) Histology was performed on the wounds on postoperative day 7, with Hematoxylin-eosin (HE) and Masson's trichrome (MT). (FIG. 33D) Scar Index was evaluated using HE stained slices. (FIG. 33E) The % area of fibrous tissue was evaluated using MT stained slices. (FIG. 33F)

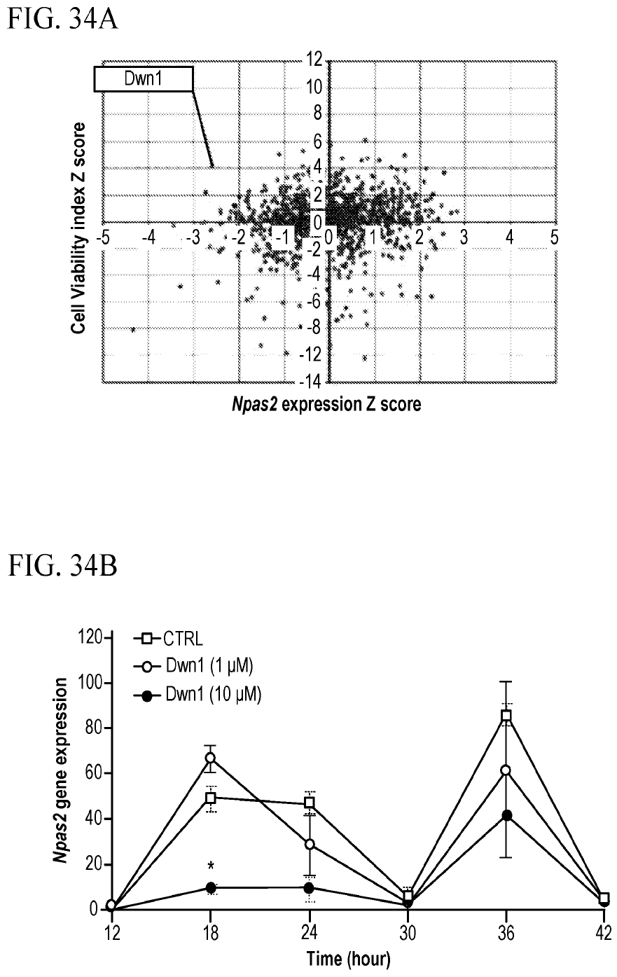

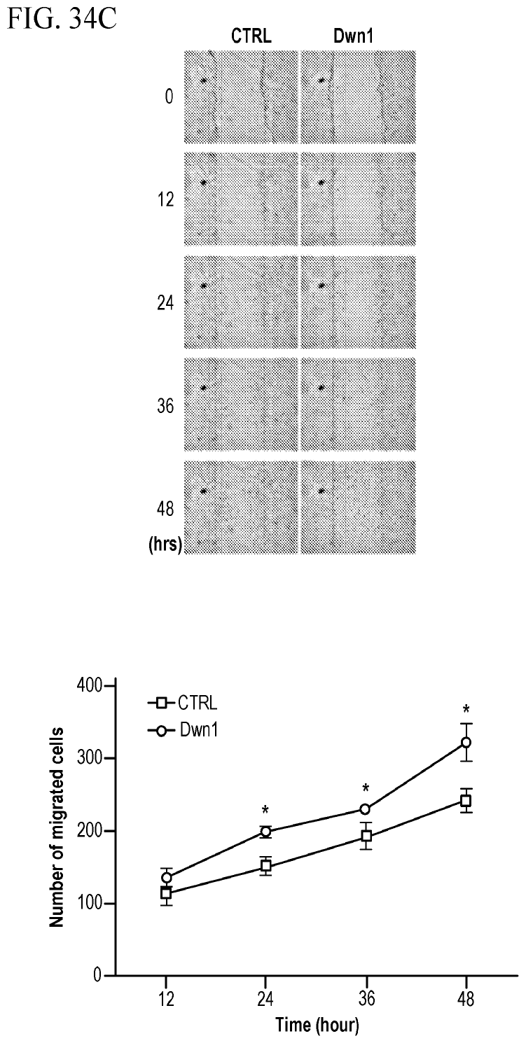

A candidate compound, Dwn1, was selected and evaluated of for Npas2-suppression on dermal fibroblast in vitro. A scatter plot of the high-through-put drug screening assay in vitro using FDA-approved compounds library in MSSR at UCLA was made. (FIG. 34A). High absolute value of negative Npas2 Z score indicates that Npas2 expression was highly downregulated (X axis). High cell viability Z score indicates that fibroblast had high viability (Y axis). Candidate compound (Dwn1) were selected with the order from high absolute value of negative product of Npas2 Z score and highest viability. Circadian Npas2 expression in murine dermal fibroblasts treated with Dwn1 (1 μM or 10 μM) was evaluated and compared to control. (FIG. 34B) An evaluation of cell migration of murine dermal fibroblasts treated with Dwn1 was performed * p <0.05. (FIG. 34C)

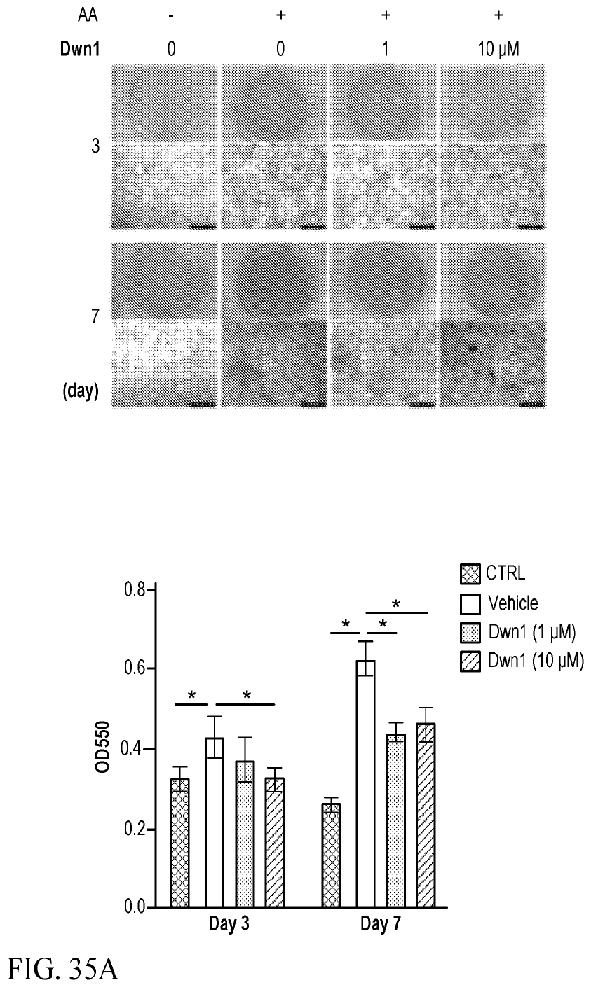

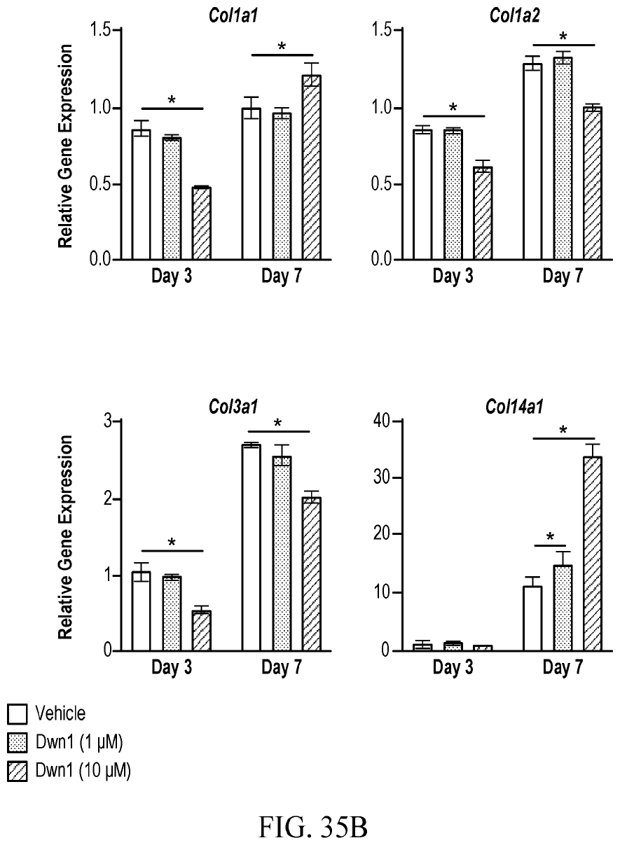

The effects of two different concentrations of Dwn1 (1 μM or 10 μM) on collagen synthesis in murine dermal fibroblast in vitro were examined Murine dermal fibroblasts were seeded into 24-well plates and cultured at 80%-90% confluency in medium supplemented with ascorbic acid (50 μg/mL) for 1, 3, and 7 days. The cells were then fixed with 10% neutral buffered formalin and stained with Picrosirius red (PolyScience, Niles, Ill.) for visualizing the collagen. AA: 1-ascorbic acid. OD: optical density. CTRL: cell treated with control medium without AA. Picrosirius red staining for fibroblasts cultured with ascorbic acid supplementation showed an increase of a positive reaction, indicating collagen fiber formation and accumulation in fibroblasts on day 7 after 1 μM or 10 μM Dwn1 treatment compared to control and 50 μg AA. (FIG. 35A) The absorption value was read in a spectrophotometer at 550 nm with a plate reader (SYHNERGY H1 plate reader) and compared by one-way ANOVA with the post hoc Holm test.

Gene expression of collagen type Col1a1, Col1a2, Col3a1, and Col14a1 was evaluated on granulation tissue and wounded tissue after 1 μM or 10 μM Dwn1 treatment or control (vehicle) treatment. (FIG. 35B) Gapdh was used as an internal control. Immunohistochemical staining of αSMA in vivo was performed at postoperative day 7 of wounds applied with vehicle or vehicle+Dwn1. Vehicle or Dwn1+vehicle was applied every 24 hours postoperatively and evaluated on day 3 and day 7 after the respective treatment.

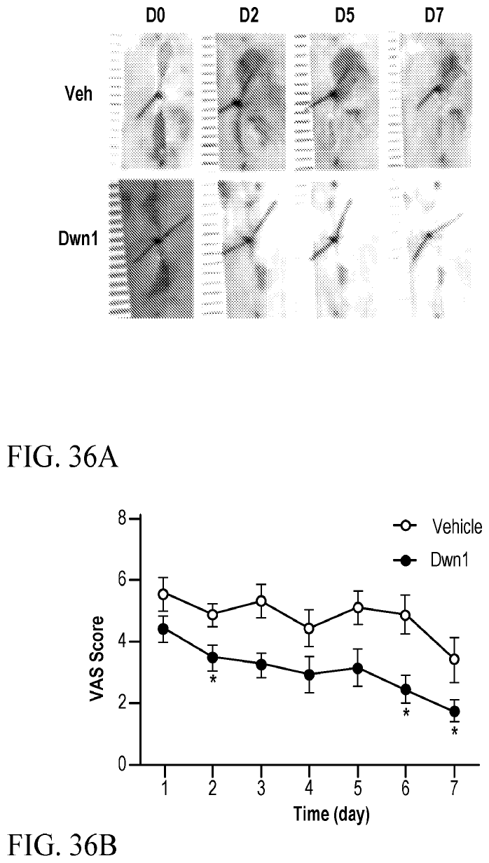

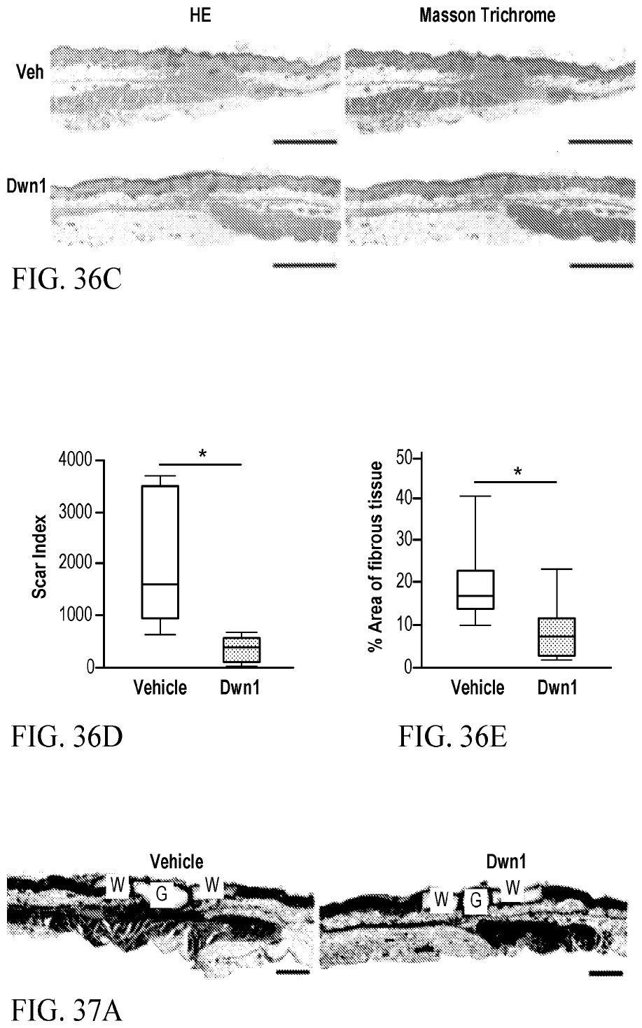

The effects of Dwn1 on the murine dorsal linear wound/scar model was evaluated. Gross image on day 0 (D0), day 2 (D2), day 5 (D5), day 7 (D7) after surgery and starting topical application of Dwn1 to the wounds. Veh: vehicle. Vehicle or Dwn1+vehicle was applied every 24 hours postoperatively. (FIG. 36A) Visual analogue score (VSA) scale of wounds applied with vehicle or vehicle+Dwn1 were evaluated. (FIG. 36B) Histological images of lateral wound/scar applied with vehicle or vehicle+Dwn1 on postoperative day 7. (FIG. 36C) Yellow dotted lines indicate granulation tissue. Left two were stained with HE and right two were stained with MT. Scale bar is 1000 μm. Scar Index was evaluated using HE stained slices. (FIG. 36D) % Area of fibrous tissue was evaluated using MT stained slices. * shows p<0.05. (FIG. 36E)

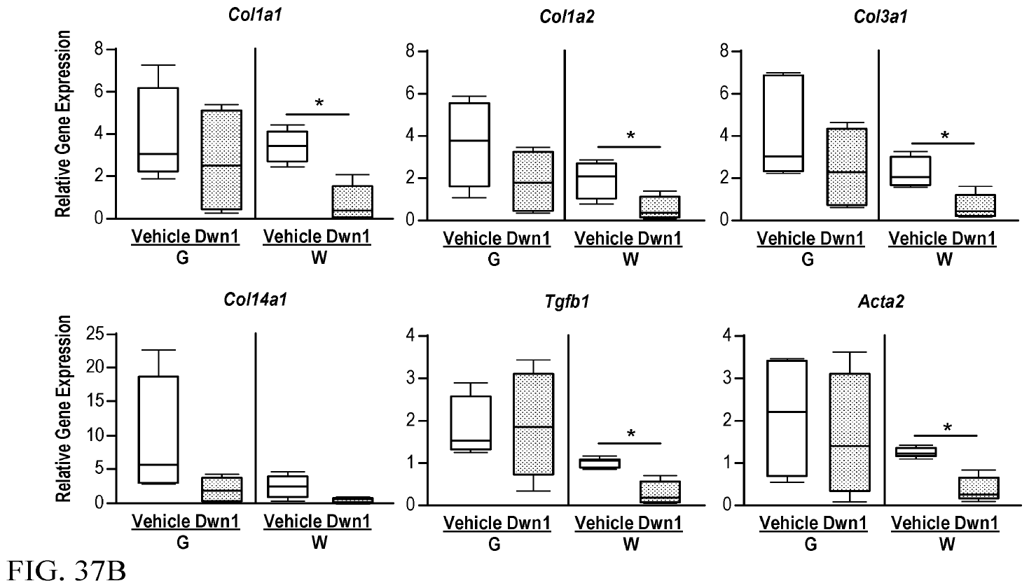

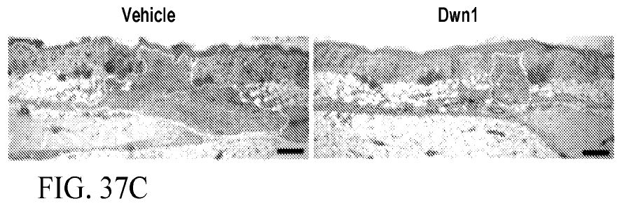

The molecular biological effects of Dwn1 on the murine dorsal linear wound/scar model were evaluated. A typical post-Laser capture microdissection (LCM) image was taken. (FIG. 37A) Slides were briefly stained with Hematoxylin and eosin before LCM. G: granulation tissue, W: wounded tissue Gene expression of Col1a1, Col1a2, Col3a1), Col14a1, Tgfβ1 and Acta2 on granulation tissue (G) and wounded tissue (W) was evaluated. (FIG. 37B) Gapdh was used as an internal control. * shows p<0.05. Immunohistochemical staining of αSMA in vivo at postoperative day 7 of wounds applied with vehicle or vehicle+Dwn1 was performed. (FIG. 37C) Yellow dotted lines indicate granulation tissue. Scale bar is 100 μm.

All patents, patent applications, and scientific publications cited herein are hereby incorporated by reference in their entirety.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for improving or accelerating wound healing in a subject comprising administering to a wound of the subject in need thereof an agent that suppresses expression of a clock gene, wherein the clock gene is neuronal PAS domain protein 2 (Npas2).

2. The method of claim 1, wherein the administering is by a route selected from topical administration, transdermal administration and subcutaneous administration.

3. The method of claim 1, wherein the wound is a dermal wound.

4. The method of claim 3, wherein the dermal wound is a periodontal wound.

5. The method of claim 4, wherein the periodontal wound comprises gingival connective tissue degeneration or alveolar bone resorption.

6. The method of claim 1, wherein the agent that suppresses expression of Npas2 accelerates human skin fibroblast migration in a cell migration assay.

7. The method of claim 1, wherein the agent is selected from a norepinephrine, dopamine and serotonin uptake inhibitor, an oxidative phosphorylation inhibitor, a cyclooxygenase-2 inhibitor, a dopamine antagonist, or a central nervous system (CNS) stimulant.

8. The method of claim 1, wherein the agent is Reserpine.

9. The method of claim 1, wherein the agent is antimycin A, niflumic acid, molindone hydrochloride and mefexamide hydrochloride.

10. The method of claim 1, wherein the agent is selected from econazole nitrate, Aceclofenac, Pravastatin, Tyloxapol, Isosorbide mononitrate, MS-1500387, (S)-(−)-Atenolo, Butenafine Hydrochloride, Aceclidine Hydrochloride, Atropine sulfate monohydrate, Trimethadione, Chlorphensin carbamate, Mafenide hydrochloride, Nifenazone, Articaine hydrochloride, Theobromine, Nifuroxazide, SAM001246626, Dropropizine (R,S), Diethylcarbamazine citrate, MS-1501214, Dolasetron mesilate, Estrone, Prednisolone, Daunorubicin hydrochloride, Cycloheximide, and Monensin sodium salt.

11. The method of claim 1, wherein the agent is a Npas2 downregulating compound selected from the group consisting of a cytoskeleton/ECM inhibitor, a hormone agonist, a nitric oxide inhibitor, an intracellular Ca++ releasor, a kinase/phosphatase inhibitor, and a kinase inhibitor.

12. The method of claim 11, wherein the cytoskeleton/ECM inhibitor is Brefeldin A, Colchicine, Podophyllotoxin or 5175348.

13. The method of claim 11, wherein the hormone agonist is AC-93253 iodide, the nitric oxide inhibitor is Diphenyleneiodonium chloride, the intracellular Ca++ releasor is THAPSIGARGIN, the kinase/phosphatase inhibitor is PD-166285 hydrate, and the kinase inhibitor is PD-173952.

14. The method of claim 2, wherein the transdermal administration is an application to the wound of deformable nanoscale vesicles encapsulating the agent.

15. The method of claim 2, wherein the transdermal administration is application to the wound of a transdermal delivery system selected from the group consisting of a microneedle coated with the agent, a solid polymer matrix having the agent incorporated therein, a transdermal patch comprising a reservoir storing the agent and a semi-permeable membrane, a transdermal gel comprising the agent dissolved therein, and a transdermal spray comprising the agent dissolved therein and a metered dose transdermal spray comprising the agent dissolved therein.

16. The method of claim 1, wherein the agent is synthetic small interfering ribonucleic acid (siRNA) designed to target mRNA of a Npas2 gene.

17. The method of claim 16, wherein the siRNA is administered by a route selected from the group consisting of microneedle array, electroporation, pressure, mechanical massage, cationic liposomes, cationic polymer-mediated delivery systems, ultrasound, conjugate delivery systems, microbubbles, liposomal bubbles, ultrasound sensitive nanobubbles, carbon nanotubes, lipid-based nanovectors, non-lipid organic-based nanovectors and inorganic nanovectors, gold nanoparticles, and gold nanorods.

18. The method of claim 16, wherein the siRNA is chemically modified at a 2′ position of a ribose sugar ring, a phosphate backbone, a nucleobase and ribose sugar, 5′ termini modification or conjugation.

19. The method of claim 18, wherein the ribose sugar ring is guanosine or uridine and the 2′ position modification is selected from the group consisting of 2′-OMe, 2′-F, 2′-O-methoxyethyl (2′-MOE).

20. The method of claim 18, wherein the phosphate backbone is modified with phosphorodithioate, triazole dimers, amide or boranophosphate.

21. The method of claim 18, wherein the nucleobase and ribose sugar modification is a 5-fluoro-2′-deoxyuridine (FdU), 2′-O-methyl phospshorodithioate (2′ O-MePS2), a lipophilic boron cluster, 3-N-[(1,12-dicarba-closo-dodecacarboran-1-yl)propan-3-yl]thymidine (C2B10H11, CB), thymidine and 5-bis(aminoethyl)-aminoethyl-2′-deoxyuridine.

22. The method of claim 18, wherein the 5′ termini modification or conjugation is palmitic acid conjugation at the 5′ terminus of the siRNA, inverted thymidine (idT) coupling to the 3′ terminus of the siRNA and topalmitic acid conjugation at the 5′ terminus, conjugation of the siRNA with cell permeable peptide (CPPs), conjugation of the siRNA with aromatic compounds selected from the group consisting of phenyl, hydroxyphenyl, naphthyl, and pyrenyl derivatives; chemical modification at a 3′ overhang region with urea/thiourea bridged aromatic compounds; polyethylene glycol (PEG) conjugation at 3′ end of sense and anti-sense strands; and cholesterol conjugation of the siRNA.

23. A method for regenerating alveolar bone comprising administering to a bone loss site of a subject in need thereof an agent that suppresses expression of Npas2.

24. The method of claim 23, wherein the administering is by a route selected from topical administration and transdermal administration.

25. The method of claim 23, wherein the wound is a dermal wound.

26. The method of claim 25, wherein the dermal wound is a periodontal wound.

27. The method of claim 26, wherein the periodontal wound comprises gingival connective tissue degeneration or alveolar bone resorption.

28. The method of claim 23, wherein the agent that suppresses expression of Npas2 accelerates human skin fibroblast migration in a cell migration assay.

29. The method of claim 23, wherein the agent is selected from a norepinephrine and serotonin uptake inhibitor, an oxidative phosphorylation inhibitor, a cyclooxygenase-2 inhibitor, a dopamine antagonist, or a central nervous system (CNS) stimulant.

30. The method of claim 23, wherein the agent is Reserpine.

31. The method of claim 23, wherein the agent is antimycin A, niflumic acid, molindone hydrochloride and mefexamide hydrochloride.

32. The method of claim 23, wherein the agent is selected from econazole nitrate, Aceclofenac, Pravastatin, Tyloxapol, Isosorbide mononitrate, MS-1500387, (S)-(−)-Atenolo, Butenafine Hydrochloride, Aceclidine Hydrochloride, Atropine sulfate monohydrate, Trimethadione, Chlorphensin carbamate, Mafenide hydrochloride, Nifenazone, Articaine hydrochloride, Theobromine, Nifuroxazide, SAM001246626, Dropropizine (R,S), Diethylcarbamazine citrate, MS-1501214, Dolasetron mesilate, Estrone, Prednisolone, Daunorubicin hydrochloride, Cycloheximide, and Monensin sodium salt.

33. The method of claim 23, wherein the agent is a Npas2 downregulating compound selected from the group consisting of a cytoskeleton/ECM inhibitor, a hormone agonist, a nitric oxide inhibitor, an intracellular Ca++ releasor, a kinase/phosphatase inhibitor, and a kinase inhibitor.

34. The method of claim 33, wherein the cytoskeleton/ECM inhibitor is Brefeldin A, Colchicine, Podophyllotoxin or 5175348.

35. The method of claim 33, wherein the hormone agonist is AC-93253 iodide, the nitric oxide inhibitor is Diphenyleneiodonium chloride, the intracellular Ca++ releasor is THAPSIGARGIN, the kinase/phosphatase inhibitor is PD-166285 hydrate, and the kinase inhibitor is PD-173952.

36. The method of claim 24, wherein the transdermal administration is by deformable nanoscale vesicles encapsulating the agent.

37. The method of claim 24, wherein the transdermal administration is application to the wound of a transdermal delivery system selected from the group consisting of a microneedle coated with the agent, a solid polymer matrix having the agent incorporated therein, a transdermal patch comprising a reservoir storing the agent and a semi-permeable membrane, a transdermal gel comprising the agent dissolved therein, and a transdermal spray comprising the agent dissolved therein and a metered dose transdermal spray comprising the agent dissolved therein.

38. The method of claim 23, wherein the agent is synthetic small interfering ribonucleic: acid (siRNA) designed to target mRNA of a Npas2 gene.

39. The method of claim 38, wherein the siRNA is administered by a route selected from the group consisting of microneedle array, electroporation, pressure, mechanical massage, cationic liposomes, cationic polymer-mediated delivery systems, ultrasound, conjugate delivery systems, microbubbles, liposomal bubbles, ultrasound sensitive nanobubbles, carbon nanotubes, lipid-based nanovectors, non-lipid organic-based nanovectors and inorganic nanovectors, gold nanoparticles, and gold nanorods.

40. The method of claim 38, wherein the siRNA is chemically modified at a 2′ position of a ribose sugar ring, a phosphate backbone, a nucleobase and ribose sugar, 5′ termini modification or conjugation.

41. The method of claim 40, wherein the ribose sugar ring is guanosine or uridine and the 2′ position modification is selected from the group consisting of 2′-OMe, 2′-F, 2′-O-methoxyethyl (2′-MOE).

42. The method of claim 40, wherein the phosphate backbone is modified with phosphorodithioate, triazole dimers, amide or boranophosphate.

43. The method of claim 40, wherein the nucleobase and ribose sugar modification is a 5-fluoro-2′-deoxyuridine (FdU), 2′-O-methyl phospshorodithioate (2′ O-MePS2), a lipophilic boron cluster, 3-N-[(1,12-dicarba-closo-dodecacarboran-1-yl)propan-3-yl]thymidine (C2B10H11, CB), thymidine and 5-bis(aminoethyl)-aminoethyl-2′-deoxyuridine.

44. The method of claim 40, wherein the 5′ termini modification or conjugation is palmitic acid conjugation at the 5′ terminus of the siRNA, inverted thymidine (idT) coupling to the 3′ terminus of the siRNA and topalmitic acid conjugation at the 5′ terminus, conjugation of the siRNA with cell permeable peptide (CPPs), conjugation of the siRNA with aromatic compounds selected from the group consisting of phenyl, hydroxyphenyl, naphthyl, and pyrenyl derivatives; chemical modification at a 3′ overhang region with urea/thiourea bridged aromatic compounds; polyethylene glycol (PEG) conjugation at 3′ end of sense and anti-sense strands; and cholesterol conjugation of the siRNA.

45. A method for regenerating connective tissue at a wound site in a subject in need thereof comprising administering to the wound a therapeutically effective amount of a Npas2 expression suppressor.

46. The method of claim 45, wherein the administering is by a route selected from topical administration and transdermal administration.

47. The method of claim 45, wherein the wound is a dermal wound.

48. The method of claim 47, wherein the dermal wound is a periodontal wound.

49. The method of claim 48, wherein the periodontal wound comprises gingival connective tissue degeneration or alveolar bone resorption.

50. The method of claim 45, wherein the agent that suppresses expression of Npas2 accelerates human skin fibroblast migration in a cell migration assay.

51. The method of claim 45, wherein the agent is selected from a norepinephrine and serotonin uptake inhibitor, an oxidative phosphorylation inhibitor, a cyclooxygenase-2 inhibitor, a dopamine antagonist, or a central nervous system (CNS) stimulant.

52. The method of claim 45, wherein the agent is Reserpine.

53. The method of claim 45, wherein the agent is antimycin A, niflumic acid, molindone hydrochloride and mefexamide hydrochloride.

54. The method of claim 45, wherein the agent is selected from econazole nitrate, Aceclofenac, Pravastatin, Tyloxapol, Isosorbide mononitrate, MS-1500387, (S)-(−)-Atenolo, Butenafine Hydrochloride, Aceclidine Hydrochloride, Atropine sulfate monohydrate, Trimethadione, Chlorphensin carbamate, Mafenide hydrochloride, Nifenazone, Articaine hydrochloride, Theobromine, Nifuroxazide, SAM001246626, Dropropizine (R,S), Diethylcarbamazine citrate, MS-1501214, Dolasetron mesilate, Estrone, Prednisolone, Daunorubicin hydrochloride, Cycloheximide, and Monensin sodium salt.

55. The method of claim 45, wherein the agent is a Npas2 downregulating compound selected from the group consisting of a cytoskeleton/ECM inhibitor, a hormone agonist, a nitric oxide inhibitor, an intracellular Ca++ releasor, a kinase/phosphatase inhibitor, and a kinase inhibitor.

56. The method of claim 55, wherein the cytoskeleton/ECM inhibitor is Brefeldin A, Colchicine, Podophyllotoxin or 5175348.

57. The method of claim 55, wherein the hormone agonist is AC-93253 iodide, the nitric oxide inhibitor is Diphenyleneiodonium chloride, the intracellular Ca++ releasor is THAPSIGARGIN, the kinase/phosphatase inhibitor is PD-166285 hydrate, and the kinase inhibitor is PD-173952.

58. The method of claim 46, wherein the transdermal administration is an application to the wound of deformable nanoscale vesicles encapsulating the agent.

59. The method of claim 46, wherein the transdermal administration is application to the wound of a transdermal delivery system selected from the group consisting of a microneedle coated with the agent, a solid polymer matrix having the agent incorporated therein, a transdermal patch comprising a reservoir storing the agent and a semi-permeable membrane, a transdermal gel comprising the agent dissolved therein, and a transdermal spray comprising the agent dissolved therein and a metered dose transdermal spray comprising the agent dissolved therein.

60. The method of claim 45, wherein the agent is synthetic small interfering ribonucleic acid (siRNA) designed to target mRNA of a Npas2 gene.

61. The method of claim 60, wherein the siRNA is administered by a route selected from the group consisting of microneedle array, electroporation, pressure, mechanical massage, cationic liposomes, cationic polymer-mediated delivery systems, ultrasound, conjugate delivery systems, microbubbles, liposomal bubbles, ultrasound sensitive nanobubbles, carbon nanotubes, lipid-based nanovectors, non-lipid organic-based nanovectors and inorganic nanovectors, gold nanoparticles, and gold nanorods.

62. The method of claim 60, wherein the siRNA is chemically modified at a 2′ position of a ribose sugar ring, a phosphate backbone, a nucleobase and ribose sugar, 5′ termini modification or conjugation.

63. The method of claim 62, wherein the ribose sugar ring is guanosine or uridine and the 2′ position modification is selected from the group consisting of 2′-OMe, 2′-F, 2′-O-methoxyethyl (2′-MOE).

64. The method of claim 62, wherein the phosphate backbone is modified with phosphorodithioate, triazole dimers, amide or boranophosphate.

65. The method of claim 62, wherein the nucleobase and ribose sugar modification is a 5-fluoro-2′-deoxyuridine (FdU), 2′-O-methyl phosphorodithioate (2′ O-MePS2), a lipophilic boron cluster, 3-N-[(1,12-dicarba-closo-dodecacarboran-1-yl)propan-3-yl]thymidine (C2B10H11, CB), thymidine and 5-bis(aminoethyl)-aminoethyl-2′-deoxyuridine.

66. The method of claim 62, wherein the 5′ termini modification or conjugation is palmitic acid conjugation at the 5′ terminus of the siRNA, inverted thymidine (idT) coupling to the 3′ terminus of the siRNA and topalmitic acid conjugation at the 5′ terminus, conjugation of the siRNA with cell permeable peptide (CPPs), conjugation of the siRNA with aromatic compounds selected from the group consisting of phenyl, hydroxyphenyl, naphthyl, and pyrenyl derivatives; chemical modification at a 3′ overhang region with urea/thiourea bridged aromatic compounds; polyethylene glycol (PEG) conjugation at 3′ end of sense and anti-sense strands; and cholesterol conjugation of the siRNA.

67. The method of claim 45, wherein the connective tissue is one or more of collagen, dermis-like collagen fibers, or bone.

68. The method of claim 45, wherein the wound site is a site of bone loss.

69. The method of claim 68, wherein the bone loss is a site of periodontitis-induced alveolar bone resorption.

70. The method of claim 45, wherein the wound site is a site of gingival connective tissue degeneration.

71. A method for decreasing wound area size comprising topically administering to an open wound site of a subject an agent that suppresses expression of Npas2.

72. The method of claim 71, wherein the administering is by a route selected from topical administration and transdermal administration.

73. The method of claim 71, wherein the wound is a dermal wound.

74. The method of claim 73, wherein the dermal wound is a periodontal wound.

75. The method of claim 74, wherein the periodontal wound comprises gingival connective tissue degeneration or alveolar bone resorption.

76. The method of claim 71, wherein the agent that suppresses expression of Npas2 accelerates human skin fibroblast migration in a cell migration assay.

77. The method of claim 71, wherein the agent is selected from a norepinephrine and serotonin uptake inhibitor, an oxidative phosphorylation inhibitor, a cyclooxygenase-2 inhibitor, a dopamine antagonist, or a central nervous system (CNS) stimulant.

78. The method of claim 71, wherein the agent is Reserpine.

79. The method of claim 71, wherein the agent is antimycin A, niflumic acid, molindone hydrochloride and mefexamide hydrochloride.

80. The method of claim 71, wherein the agent is selected from econazole nitrate, Aceclofenac, Pravastatin, Tyloxapol, Isosorbide mononitrate, MS-1500387, (S)-(−)-Atenolo, Butenafine Hydrochloride, Aceclidine Hydrochloride, Atropine sulfate monohydrate, Trimethadione, Chlorphensin carbamate, Mafenide hydrochloride, Nifenazone, Articaine hydrochloride, Theobromine, Nifuroxazide, SAM001246626, Dropropizine (R,S), Diethylcarbamazine citrate, MS-1501214, Dolasetron mesilate, Estrone, Prednisolone, Daunorubicin hydrochloride, Cycloheximide, and Monensin sodium salt.

81. The method of claim 71, wherein the agent is a Npas2 downregulating compound selected from the group consisting of a cytoskeleton/ECM inhibitor, a hormone agonist, a nitric oxide inhibitor, an intracellular Ca++ releasor, a kinase/phosphatase inhibitor, and a kinase inhibitor.

82. The method of claim 81, wherein the cytoskeleton/ECM inhibitor is Brefeldin A, Colchicine, Podophyllotoxin or 5175348.

83. The method of claim 81, wherein the hormone agonist is AC-93253 iodide, the nitric oxide inhibitor is Diphenyleneiodonium chloride, the intracellular Ca++ releasor is THAPSIGARGIN, the kinase/phosphatase inhibitor is PD-166285 hydrate, and the kinase inhibitor is PD-173952.

84. The method of claim 72, wherein the transdermal administration is an application to the wound of deformable nanoscale vesicles encapsulating the agent.

85. The method of claim 72, wherein the transdermal administration is application to the wound of a transdermal delivery system selected from the group consisting of a microneedle coated with the agent, a solid polymer matrix having the agent incorporated therein, a transdermal patch comprising a reservoir storing the agent and a semi-permeable membrane, a transdermal gel comprising the agent dissolved therein, and a transdermal spray comprising the agent dissolved therein and a metered dose transdermal spray comprising the agent dissolved therein.

86. The method of claim 71, wherein the agent is synthetic small interfering ribonucleic acid (siRNA) designed to target mRNA of a Npas2 gene.

87. The method of claim 86, wherein the siRNA is administered by a route selected from the group consisting of microneedle array, electroporation, pressure, mechanical massage, cationic liposomes, cationic polymer-mediated delivery systems, ultrasound, conjugate delivery systems, microbubbles, liposomal bubbles, ultrasound sensitive nanobubbles, carbon nanotubes, lipid-based nanovectors, non-lipid organic-based nanovectors and inorganic nanovectors, gold nanoparticles, and gold nanorods.

88. The method of claim 86, wherein the siRNA is chemically modified at a 2′ position of a ribose sugar ring, a phosphate backbone, a nucleobase and ribose sugar, 5′ termini modification or conjugation.

89. The method of claim 88, wherein the ribose sugar ring is guanosine or uridine and the 2′ position modification is selected from the group consisting of 2′-OMe, 2′-F, 2′-O-methoxyethyl (2′-MOE).

90. The method of claim 88, wherein the phosphate backbone is modified with phosphorodithioate, triazole dimers, amide or boranophosphate.

91. The method of claim 88, wherein the nucleobase and ribose sugar modification is a 5-fluoro-2′-deoxyuridine (FdU), 2′-O-methyl phosphorodithioate (2′ O-MePS2), a lipophilic boron cluster, 3-N-[(1,12-dicarba-closo-dodecacarboran-1-yl)propan-3-yl]thymidine (C2B10H11, CB), thymidine and 5-bis(aminoethyl)-aminoethyl-2′-deoxyuridine.

92. The method of claim 88, wherein the 5′ termini modification or conjugation is palmitic acid conjugation at the 5′ terminus of the siRNA, inverted thymidine (idT) coupling to the 3′ terminus of the siRNA and topalmitic acid conjugation at the 5′ terminus, conjugation of the siRNA with cell permeable peptide (CPPs), conjugation of the siRNA with aromatic compounds selected from the group consisting of phenyl, hydroxyphenyl, naphthyl, and pyrenyl derivatives; chemical modification at a 3′ overhang region with urea/thiourea bridged aromatic compounds; polyethylene glycol (PEG) conjugation at 3′ end of sense and anti-sense strands; and cholesterol conjugation of the siRNA.

93. The method of claim 71, wherein the open wound site comprises connective tissue selected from one or more of collagen, dermis-like collagen fibers, or bone.

94. The method of claim 71, wherein the open wound site is a site of bone loss.

95. The method of claim 94, wherein the bone loss is a site of periodontitis-induced alveolar bone resorption.

96. The method of claim 71, wherein the open wound site is a site of gingival connective tissue degeneration.
